(19) United States
(12) Patent Application Publication
(10) Pub. No.: US 2023/0235347 A1
(43) Pub. Date: Jul. 27, 2023

(54) Improved Camelina Plants and Plant Oil, and Uses Thereof

(71) Applicants: Kansas State University Research Foundation (KSURF), Manhattan, KS (US); NUtech Ventures, Lincoln, NE (US)

(72) Inventors: Nicholas G. NEUMANN, Manhattan, KS (US); Tara J. NAZARENUS, Lincoln, NE (US); Jose A. AZNAR-MORENO, Manhattan, KS (US); Timothy P. DURRETT, Manhattan, KS (US); Edgar B. CAHOON, Lincoln, NE (US)

(21) Appl. No.: 17/770,466

(22) PCT Filed: Oct. 6, 2021
(86) PCT No.: PCT/US2021/053744

§ 371 Date: Apr. 20, 2022

Related U.S. Application Data
(60) Provisional application No. 63/089,189, filed on Oct. 8, 2020.
(60) Provisional application No. 63/114,178, filed on Nov. 16, 2020.

(51) Int. Cl. C12N 15/82; C12N 9/02; C12N 9/10

(52) U.S. Cl. CPC C12N 15/8247; C12N 9/0071; C12Y 114/19003; C12N 9/1029; C12Y 203/01199; C12Y 203/01179

(57) ABSTRACT

The present invention provides isolated FAD2, FAD3, FAB1 and FAE1 genes and FAD2, FAD3, FAB1 and FAE1 protein sequences of Camelina species, e.g., Camelina sativa, mutations in Camelina FAD2, FAD3, FAB1 and FAE1 genes, and methods of using the same. In addition, methods of altering Camelina seed composition and/or improving Camelina seed oil quality are disclosed. Furthermore, methods of breeding Camelina cultivars to produce plants having altered or improved seed oil and/or meal quality are provided.

### CROSS-REFERENCE TO RELATED APPLICATIONS

This application claims priority to U.S. Provisional Patent Application Nos. 63/089,189, filed Oct. 8, 2020, and 63/114,178, filed Nov. 16, 2020; both of which are incorporated herein by reference in their entirety.

### GOVERNMENT SUPPORT

This invention was made with government support under IOS1339385 and IOS1444612 awarded by the National Science Foundation, under DE-SC0012459 awarded by the United States Department of Energy, and under NI17HFPXXXXXG047, NI18HFPXXXXXG045, NI19HFPXXXXXG019, NI20HFPXXXXXG055, NI21HFPXXXXXG004, and NI17HMFPXXXXG026 awarded by the United States Department of Agriculture, National Institute of Food and Agriculture. The government has certain rights in the invention.

### SEQUENCE LISTING

The Sequence Listing for this application is labeled “SeqList-190ct20_ST25.txt,” which was created on Oct. 19, 2020, and is 171 KB. The Sequence Listing is incorporated herein by reference in its entirety.

### BACKGROUND

The primary component of vegetable oil derived from different oilseed crops is triacylglycerol (TAG), molecules with three fatty acids esterified to a glycerol backbone. Vegetable oil has mostly been produced for human consumption, but over the last few decades, an increasing amount of this valuable agricultural commodity has been used as a source of biodiesel, or for other industrial applications. This increased demand has led to the identification of new TAG sources, particularly for non-food applications.

Camelina (Camelina sativa), a member of the Brassicaceae family, has emerged as one such suitable industrial oil seed crop. Camelina seed oil content ranges from 32-41% of seed weight, with the fatty acid profile being dominated by the polyunsaturated fatty acids 18:2 and 18:3.

Camelina can be grown without displacing other crops. In this regard, it is suited for cultivation on land not typically used for food crop production due to its productivity with limited rainfall and minimal soil fertility inputs. Under drought conditions, camelina achieves higher seed yields compared to other Brassicaceae oil seed crops. Also, camelina has a relatively short growing season (85-100 days), and possesses winter and spring varieties, making this crop very attractive for integrating into existing agricultural practices as a relay crop. Camelina can be grown as a rotation crop during fallow years with wheat and other dryland cereals, without affecting the yield of these crops, thus making available roughly 5-7 million acres of fallow land each year, allowing the production of 750,000,000 to 1 billion gallons of camelina oil per year.

Camelina oil has also been used as a feedstock for jet fuel production through conversion methods such as hydroprocessing, and life cycle analyses show that production and use of jet fuel from camelina results in 75% lower greenhouse gas emissions relative to petroleum-derived fuel.

In addition to its positive agronomic and sustainability traits, camelina also benefits from the availability of a variety of genetic and biotechnology tools with which to further improve different traits. These include abundant genomic and transcriptomic resources and a facile Agrobacterium floral-infiltration transformation system that allows for the overexpression of both endogenous and exogenous genes, as well as targeting gene expression through methods such as CRISPR and RNA-interference.

Further, camelina's close relationship with Arabidopsis thaliana facilitates the straightforward transfer of the wealth of knowledge of the model species into this oilseed crop. Camelina's hexaploid genome also offers advantages for successful mutational breeding, allowing knockout of one or two of the three homeologous genes that may be essential or compromise agronomic performance when disrupted in diploid oilseeds such as pennycress.

Despite its numerous positive attributes as a biofuel feedstock, the need remains to improve camelina's agronomic and seed quality traits. In particular, changes in fatty acid composition will greatly improve the utility of camelina oil, which is currently prone to oxidation due to its high content of polyunsaturated fatty acids, making it less suitable for certain applications.

For example, biodiesel derived from camelina possesses a lower oil stability index (OSI) compared to biodiesel from other feedstocks. The quality of a biodiesel, regardless of its source, is dependent upon the fatty acid methyl ester (FAME) composition, which affects cold flow and oxidative stability. For instance, saturated FAMEs have poor cold flow properties because they can form crystals at lower temperatures, while the FAMEs from polyunsaturated fatty acids remain in solution at colder temperatures, and thus have good cold flow properties.

In contrast, the relationship between saturation and oxidative stability is exactly opposite that of cold flow. Fatty acid saturation is positively correlated with oxidative stability; saturated fatty acids have the best oxidative stability and fatty acids with 2 or greater double bonds have increasing oxidative instability.

Additionally, polyunsaturated FAMEs can result in increased NOx emissions, e.g., NO, NO₂ et al. and thus affect the production of a greenhouse gas. Saturated fatty acids and very long-chain fatty acids (VLCFA) with chain-lengths ≥C20 are also targets for enhancement of biofuel functionality to address deficiencies in pour-point and other qualities (Durrett et al., 2008).

The naturally occurring oil composition of C. saliva negatively affects its biofuel properties. Polyunsaturated fatty acids such as linoleic (18:2) and alpha-linolenic (18:3) acids account for 52.1-54.7% of C. sativa seed oil (Ni Eidhin, Burke et al. 2003; Abramovic and Abram 2005). This likely accounts for the low oxidative stability of C. sativa FAMEs. C. sativa seeds also contain 21.4-22.4% VLCFA, of which 11-eicosenoic acid (20:1) at 14.9-16.2% are especially abundant, likely resulting in the high distillation temperature of the FAMEs. Oleic acid (18:1) accounts for 14.0-17.4% of C. sativa seed oil (Budin, Breene et al. 1995; Zubr 2002; Ni Eidhin, Burke et al. 2003; Abramovic and Abram 2005).

Mutational breeding and biotechnological approaches can be applied to address deficiencies in camelina seed oil quality. The key target genes for these efforts include genes for fatty acid desaturases that control polyunsaturated fatty acid production (FIG. 1), most notably genes for FATTY ACID DESATURASE2 (FAD2) that forms linoleic acid (18:2Δ9,12) by A12 desaturation of oleic acid linked to phosphatidyleholine (PC) and FATTY ACID DESATURASE3 (FAD3) for α-linolenic acid (18:3Δ9,12,15) production by subsequent Δ15 desaturation of linoleic acid bound to PC (Arondel et al., 1992; Okuley et al., 1994; Yadav et al., 1993).

In addition, carbon chain extension of oleic acid to the VLCFAs eicosenoic acid (20:1Δ11) and docosenoic (or erucic) acid (22:1Δ13) is initiated by the FATTY ACID ELONGASE1 (FAE1)-encoded β-ketoacyl-CoA synthase, and mutation of FAE1 can provide further increases in seed TAG oleic acid content. Furthermore, the relative amounts of the saturated fatty acids palmitic acid (16:0) and stearic acid (18:0) are regulated by β-ketoacyl-acyl carrier protein (ACP) synthase II (KASII) in plastid-localized fatty acid biosynthesis encoded by the FATTYACID BIOSYNTHESIS1 (FAB1) gene. This enzyme initiates the two-carbon elongation of 16:0-ACP for formation of 18:0-ACP (Carlsson et al., 2002; Wu et al., 1994).

Mutations to FAD2 and FAE1 have been previously described in U.S. Pat. No. 9,035,131, which is incorporated herein by reference in its entirety.

As an oilseed crop in the Brassicaceae family, Camelina sativa has inspired renewed interest due to its potential for biofuels and biolubricant applications. Thus, there is a need to characterize the genes in the fatty acid biosynthesis pathway, identify the causative mutations in specific homeologs of genes in the fatty acid biosynthesis pathway, and generate mutants with alteration of seed oil fatty acid composition and enhanced oil oxidative stability.

### SUMMARY OF THE INVENTION

The subject invention provides materials and methods for generating improved populations of camelina (Camelina saliva) with altered seed oil composition. In specific embodiments, the subject invention provides methods for manipulating the fatty acid biosynthesis pathway in Camelina sativa, allowing for modifying the oil composition of this biofuel crop.

C. saliva is an allohexaploid whose oil composition can be influenced by one or more functional copies of FAE1, FAD3, FAB1 and FAD2. This allows highly specialized blends of oil to be produced from C. saliva with mutations in, for example, FAE1, FAD3, FAB1 and/or FAD2, thereby greatly increasing the utility of this crop.

Specifically, the present invention provides methods of improving Camelina fatty acids composition by disrupting and/or altering one, two, or all three copies of one or more fatty acid synthesis genes in Camelina. In one embodiment, the methods comprise introducing mutations into one or more fatty acid synthesis genes.

In a further embodiment, the present invention provides improved Camelina sativa fatty acid compositions. In preferred embodiments, the compositions have increased levels of 18:1 fatty acid (oleic acid) and decreased polyunsaturated fatty acids and long chain fatty acids. In accordance with the subject invention, one way by which 18:1 is increased and/or polyunsaturated fatty acids and long chain fatty acids are decreased is by lowering the activity of FAD2, FAD3 and of FAE1, and/or alter the activity of KASII.

In some embodiments, the methods comprise introducing mutations into one or more FAD2 genes (e.g., FAD2 A, FAD2 B, and FAD2 C), one or more FAD3 genes (e.g., FAD3 A, FAD3 B, and FAD3 C), and/or one or more FAE1 genes (e.g., FAE1 A, FAE1 B, and FAE1 C) of Camelina. In one embodiment, the methods further comprise introducing one or more mutations into one or more FAB1 genes (e.g., FAB1 A, FAB1 B, and FAB1 C) of Camelina.

In some embodiments, mutations in one or more copies of FAD2 genes, mutations in one or more copies of FAD3 genes, and/or mutations in one or more copies of FAE1 genes are integrated together to create mutant plants with double, triple, quadruple or more mutations. In a further embodiment, mutations in one or more copies of FAB1 genes may also be integrated into the mutant plants. In a specific embodiment, the mutant is a quadruple mutant, e.g., afae1c/fad2a/fae1a/fad3a quadruple mutant.

In one embodiment, the invention provides mutants in FAD2 A, FAD2 B, FAD2 C, FAD3 A, FAD3 B, FAD3 C, FAE1 A, FAE1 B, and/or FAE1 C genes of Camelina. The present invention also provides mutants in FAB1 A, FAB1 B, and/or FAB1 C genes of Camelina. In specific embodiments, the mutations are in the FAE1C, FAD2A, FAD3A, and/or FAE1A genes. In a preferred embodiment, the FAE1C gene of Camelina comprises a C625T mutation, the FAE1A gene of Camelina comprises a C422T mutation, the FAD2A gene of Camelina comprises a G449A mutation and/or the FAD3A gene of Camelina comprises a G301A mutation.

In further embodiments, the present invention provides mutants in FAD2 A, FAD2 B, FAD2 C, FAD3 A, FAD3 B, FAD3 C, FAE1 A, FAE1 B, and/or FAE1 C proteins of Camelina. The present invention also provides mutants in FAB1 A, FAB1 B, and/or FAB1 C proteins of Camelina. In specific embodiments, the mutations are in the FAE1C, FAD2A, FAD3A, and/or FAE1A proteins. In a preferred embodiment, the FAE1C protein of Camelina comprise a R209* mutation, the FAE1A protein of Camelina comprises a P141L mutation, the FAD2A protein of Camelina comprises a G150E mutation, and/or the FAD3A protein of Camelina comprises a G101S mutation.

In one embodiment, the present invention provides a gene, or a chimeric gene, comprising the isolated nucleic acid sequence of any one of the polynucleotides described herein, operably linked to suitable regulatory sequences.

In one embodiment, the present invention provides a transformed host cell comprising the gene as described herein. In certain embodiments, the host cell is selected from bacteria, yeasts, fungi, algae, animals, and plants.

In another embodiment, the present invention provides a plant, plant part, plant tissue, or plant cell comprising in its genome one or more genes as described herein. In a specific embodiment, the plant is a Camelina plant.

In certain embodiments, the Camelina plant, plant part, plant tissue, or plant cell has an increased level of 18:1 fatty acid, a reduced level of 20:1 fatty acid, a reduced level of 18:2 fatty acid and/or a reduced level of 18:3 fatty acid compared to the wild type.

In one embodiment, the present invention provides a plant seed obtained from the plants described herein, wherein the plants comprise in their genomes one or more genes as described herein.

In one embodiment, the present invention provides Camelina oil, or oil composition, obtained from the seeds of a Camelina plant comprising the one or more genes described herein.

In a further embodiment, the present invention provides meals made from Camelina plants comprising the one or more genes described herein, one or more genes with mutations as described herein, or one or more chimeric genes as described herein. In some embodiments, the meal is a byproduct of the extraction of the oil from said Camelina seeds.

### BRIEF DESCRIPTION OF THE DRAWINGS

FIG. 1 shows a simplified overview of fatty acid synthesis and desaturation pathways in developing seeds, with a focus on enzymes relevant to this study. ACP, acyl carrier protein; CoA, Coenzyme A; ER, endoplasmic reticulum; FAS, fatty acid synthase complex; FAE1, FATTY ACID ELONGASE1; FAD, FATTY ACID DESATURASE; KASII, β-Ketoacyl-ACP synthase II; VLCFA, very long chain fatty acid; PC, phosphatidylcholine; 16:0, palmitic acid; 18:0, stearic acid; 18:1, oleic acid; 18:2, linoleic acid; 18:3, α-linolenic acid.

FIG. 2 shows the wild type and mutant plants flowering in the greenhouse. From left to right; wild type (Ames 1043), CS2901 (fae1c), CS2362 (fad2a), CS2864 (fad3a, fae1a), CS2901×CS2362 (fae1c, fad2a) and CS2901/CS2362×CS2864 (fae1a, fae1c, fad2a, fad3a).

FIGS. 3A-3B show the fatty acid composition (A) and content (B) of M₄ seed from camelina mutant lines. Fatty acid composition is expressed as the weight percentage (wt %) of each fatty acid relative to the total weight of fatty acids. Fatty acid content is expressed as the wt % of total fatty acids relative to the weight of the seed sample. Data represents the mean±standard deviation of at least three biological replicates. *, P<0.05, ** P<0.01 (Student's t test).

FIGS. 4A-4B show alignment of Arabidopsis thaliana and Camelina saliva FAE1 nucleotide sequences near the region of ethyl methanesulfonate (EMS) induced single nucleotide polymorphism (SNP) in csfae1a (A) and csfae1c (B) (SEQ ID NOs: 87-93), and amino acid alignment (C) of CsFAE1A (SEQ ID NO: 1), csfae1a (SEQ ID NO: 2), CsFAE1B (SEQ ID NO: 3), CsFAE1C (SEQ ID NO: 4), csfae1c (SEQ ID NO: 5), and AtFAE1 (SEQ ID NO: 6) with an amino acid change of proline to leucine in csfae1a and arginine to a stop codon in csfae1c. A region highly conserved among condensing enzymes in VLCFA biosynthesis is indicated by a red box. Identical nucleotides and amino acids are shaded.

FIGS. 5A-5D show mutations in fatty acid synthesis genes result in changes to conserved amino acids. Amino acid alignments of regions of the FAE1C (A), FAD2A (B), FAD3A and FAE1A (C), and KASII (D) homeologs from Camelina saliva with orthologs from Arabidopsis thaliana, Oryza saliva, Sorghum bicolor, Chlamydomonas reinhardtii, Escherichia coli and Staphylococcus aureus. Black triangles indicate the mutations present in the alleles in the different mutant lines. Conserved amino acids are shaded black and similar amino acids are shaded gray. Red boxed regions represent conserved histidine boxes present in fatty acid desaturases. GenBank accession numbers for the proteins used in the alignments are located in Table 3.

FIGS. 6A-6B show the alignment of Arabidopsis thaliana and Camelina saliva FAD2 nucleotide sequences near the region of EMS induced SNP in csfad2a (A) (SEQ ID NOs: 94-95) and amino acid alignment (B) of CsFAD2A (SEQ ID NO: 30), csfad2a (SEQ ID NO: 31), CsFAD2B (SEQ ID NO: 32), CsFAD2C (SEQ ID NO: 33), and AtFAD2 (SEQ ID NO: 34) with an amino acid change of glycine to glutamic acid. The three conserved histidine boxes characteristic of all membrane bound desaturases are indicated by red boxes. The four transmembrane domains and ER localization signal are indicated. Identical nucleotides and amino acids are shaded.

FIGS. 7A-7B show alignment of Arabidopsis thaliana and Camelina saliva FAD3 nucleotide sequences near the region of EMS induced SNP in csfad3a (A) (SEQ ID NOs: 96-98) and amino acid alignment (B) of CsFAD3C (SEQ ID NO: 35), CsFAD3B (SEQ ID NO: 36), CsFAD3A (SEQ ID NO: 37), csfad3a (SEQ ID NO: 38), and AtFAD3 (SEQ ID NO: 39) with an amino acid change of glycine to serine. The three conserved histidine boxes characteristic of all membrane bound desaturases are indicated by red boxes. The four transmembrane domains and ER localization signal are indicated. Identical nucleotides and amino acids are shaded.

FIGS. 8A-8B show the alignment of Arabidopsis thaliana and Camelina saliva FAB1 nucleotide CDS sequences near the region of EMS induced SNP in csfab1c (A) and amino acid alignment (B) of CsFAB1A (SEQ ID NO: 44), CsFAB1B (SEQ ID NO: 45), CsFAB1C (SEQ ID NO: 46), esfab1c (SEQ ID NO: 47), and AtFAB1 (SEQ ID NO: 48) with an amino acid change of proline to leucine at 269. Black triangles indicate the mutations present in the alleles in the different mutant lines. Black highlighted regions represent conserved amino acids and grey highlighted regions represent similar amino acids.

FIGS. 9A-9F show Camelina mutant alleles being non-functional when expressed in yeast. Gas chromatograms showing the fatty acid composition of total lipids extracted from yeast transformed with wild type or mutant alleles of FAE1A (A), FAE1C (B), FAD2A (C) or FAD3A (D). For A, B and C, pYES2 was used as the empty vector (EV) controls (E). For D, the empty vector used was pESC-URA (F) and FAD2A was co-expressed with FAD3A or fad3a.

FIGS. 10A-10B show the fab1c allele segregating with high palmitate content. (A) dCAPS genotyping to detect the fab1c allele in a segregating F₂ population resulting from the backcrossing of CS1996. Black triangles indicate the absence of the fab1c allele. (B) Palmitate (16:0) content of the F₃ seed derived from the genotyped plants in (A), expressed as the weight percentage (wt %) of 16:0 relative to the total weight of fatty acids. Dark purple bars represent the CS1996 parent line, light purple bars indicated F₂ plants possessing a fab1c allele, purple-grey striped bars represent F₂ plants with a wild-type genotype for FAB1, and grey bars represent wild-type (Ames 1043) plants. Arrows indicate which lines were further genotyped in the F₃ generation to confirm homozygosity or heterozygosity.

FIG. 11 shows the genotyping of F₃ plants from fab1c backcrosses. Shown are dCAPs marker assays to detect the fab1c mutant allele in the progeny of the F₂ plants #16, #2 and #4 resulting from the backcross of the CS1996 mutant line. Black triangles indicate the absence of the fab1c allele.

FIGS. 12A-12D show complementation of fatty acid biosynthesis gene mutants. Fatty acid composition of seeds from Camelina sativa wild type, CS2864 (fad3a/fae1a) and CS2864 transformed with pBinGlyRed3/FAE1A (A), CS2901 (fae1c) with pBinGlyRed3/FAE1C (B), CS2362 (fad2a) with pBinGlyRed3/FAD2A (C) and CS2864 with pBinGlyRed3 (D). The results shown are 20:1+22:1 composition (as weight percent of total fatty acids) of wild type, CS2864 parent and five independent T₁ seeds (A), 20:1+22:1 composition of wild type, CS2901 parent and five independent T₁ seeds (B), 18:2+18:3 composition of wild type, CS2362 parent and five independent T₁ seeds (C), and 18:3 composition of wild type, CS2864 parent and five independent T₁ seeds (D).

FIGS. 13A-13B show genotyping to lines with mutations in multiple fatty acid synthesis genes. (A) Genotyping of the F₃ progeny resulting from crossing fae1c (CS2901) with fad2a (CS2362) using a dCAPS assay to detect the fae1c allele and a CAPS assay for the fad2a allele. (B) Genotyping of the F₅ progeny resulting from crossing fae1c/fad2a with fae1a/fad3a (CS2864) using a dCAPS assay to detect the fae1c allele, a CAPS assay for the fad2a allele, a CAPS assay for the fae1a allele and a dCAPS assay for the fad3a allele. +/+, +/− and −/− indicate control plants with wildtype, heterozygous or homozygous mutant genotypes for each specific allele.

FIGS. 14A-14C show combining mutations resulting in oil with increased oxidative stability. Fatty acid composition (A), oxidative stability index (B) and fatty acid content (C) of seeds from camelina lines created by crossing mutant plants to combine different loss of function alleles. Data represents the mean±standard deviation of at least three biological replicates. *, P<0.05, ** P<0.01 (Student's t test).

### BRIEF DESCRIPTION OF SEQUENCES

SEQ ID NO: 1 is an amino acid sequence of CsFAE1A contemplated for use according to the subject invention.

SEQ ID NO: 2 is an amino acid sequence of csfae1a contemplated for use according to the subject invention.

SEQ ID NO: 3 is an amino acid sequence of CsFAE1B contemplated for use according to the subject invention.

SEQ ID NO: 4 is an amino acid sequence of CsFAE1C contemplated for use according to the subject invention.

SEQ ID NO: 5 is an amino acid sequence of csfae1c contemplated for use according to the subject invention.

SEQ ID NO: 6 is an amino acid sequence of AtFAE1 contemplated for use according to the subject invention.

SEQ ID NO: 7 is the amino acid sequence from position 200 to position 225 of Camelina sativa FAE1C contemplated for use according to the subject invention.

SEQ ID NO: 8 is the amino acid sequence from position 201 to position 226 of Arabidopsis thaliana FAE1.

SEQ ID NO: 9 is the amino acid sequence from position 214 to position 239 of Oryza sativa FAE1.

SEQ ID NO: 10 is the amino acid sequence from position 221 to position 246 of Sorghum bicolor FAE1 SEQ ID NO: 11 is the amino acid sequence from position 234 to position 257 of Chlamydomonas reinhardtii FAE1.

SEQ ID NO: 12 is the amino acid sequence from position 122 to position 157 of Camelina sativa FAD2A, from position 121 to position 156 of Arabidopsis thaliana FAD2, and from position 138 to position 163 of Oryza sativa DES2 contemplated for use according to the subject invention.

SEQ ID NO: 13 is the amino acid sequence from position 136 to position 161 of of Sorghum bicolor DES2 SEQ ID NO: 14 is the amino acid sequence from position 134 to position 159 of Chlamydomonas reinhardtii FAD2.

SEQ ID NO: 15 is the amino acid sequence from position 86 to position 112 of Camelina sativa FAD3A contemplated for use according to the subject invention.

SEQ ID NO: 16 is the amino acid sequence from position 85 to position 111 of Arabidopsis thaliana FAD3.

SEQ ID NO: 17 is the amino acid sequence from position 160 to position 186 of Oryza sativa FAD7.

SEQ ID NO: 18 is the amino acid sequence from position 87 to position 113 of Sorghum bicolor DES3.

SEQ ID NO: 19 is the amino acid sequence from position 113 to position 139 of Chlamydomonas reinhardtii FAD3.

SEQ ID NO: 20 is the amino acid sequence from position 130 to position 156 of Camelina sativa FAE1A contemplated for use according to the subject invention.

SEQ ID NO: 21 is the amino acid sequence from position 131 to position 157 of Arabidopsis thaliana FAE1.

SEQ ID NO: 22 is the amino acid sequence from position 144 to position 170 of Oryza saliva FAE1.

SEQ ID NO: 23 is the amino acid sequence from position 151 to position 177 of Sorghum bicolor FAE1.

SEQ ID NO: 24 is the amino acid sequence from position 162 to position 188 of Chlamydomonas reinhardtii FAE1.

SEQ ID NO: 25 is the amino acid sequence from position 254 to position 279 of Camelina saliva FAB1C and from position 249 to position 274 of Arabidopsis thaliana FAB1.

SEQ ID NO: 26 is the amino acid sequence from position 215 to position 240 of Oryza saliva FAB1 and from position 204 to position 229 of Sorghum bicolor FAB1.

SEQ ID NO: 27 is the amino acid sequence from position 166 to position 192 of Chlamydomonas reinhardtii KAS2.

SEQ ID NO: 28 is the amino acid sequence from position 120 to position 146 of Escherichia coli FAB1.

SEQ ID NO: 29 is the amino acid sequence from position 121 to position 147 of Staphylococcus aureus FAB1.

SEQ ID NO: 30 is an amino acid sequence of CsFAD2A contemplated for use according to the subject invention.

SEQ ID NO: 31 is an amino acid sequence of csfad2a contemplated for use according to the subject invention.

SEQ ID NO: 32 is an amino acid sequence of CsFAD2B contemplated for use according to the subject invention.

SEQ ID NO: 33 is an amino acid sequence of CsFAD2C contemplated for use according to the subject invention.

SEQ ID NO: 34 is an amino acid sequence of AtFAD2 contemplated for use according to the subject invention.

SEQ ID NO: 35 is an amino acid sequence of CsFAD3C contemplated for use according to the subject invention.

SEQ ID NO: 36 is an amino acid sequence of CsFAD3B contemplated for use according to the subject invention.

SEQ ID NO: 37 is an amino acid sequence of CsFAD3A contemplated for use according to the subject invention.

SEQ ID NO: 38 is an amino acid sequence of csfad3a contemplated for use according to the subject invention.

SEQ ID NO: 39 is an amino acid sequence of AtFAD3 contemplated for use according to the subject invention.

SEQ ID NO: 40 is the nucleotide sequence from position 795 to position 835 of Camelina saliva FAB1A contemplated for use according to the subject invention.

SEQ ID NO: 41 is the nucleotide sequence from position 804 to position 844 of Camelina sativa FAB1B and from position 786 to position 826 of Camelina sativa FAB1C contemplated for use according to the subject invention.

SEQ ID NO: 42 is the nucleotide sequence from position 785 to position 826 of Camelina saliva fab1c contemplated for use according to the subject invention.

SEQ ID NO: 43 is the nucleotide sequence from position 759 to position 799 of Arabidopsis thaliana FAB1.

SEQ ID NO: 44 is an amino acid sequence of CsFAB1A contemplated for use according to the subject invention.

SEQ ID NO: 45 is an amino acid sequence of CsFAB1B contemplated for use according to the subject invention.

SEQ ID NO: 46 is an amino acid sequence of CsFAB1C contemplated for use according to the subject invention.

SEQ ID NO: 47 is an amino acid sequence of csfab1c contemplated for use according to the subject invention.

SEQ ID NO: 48 is an amino acid sequence of AtFAB1 contemplated for use according to the subject invention.

SEQ ID NO: 49 is the sequence of a forward primer for FAB1 contemplated for use according to the subject invention.

SEQ ID NO: 50 is the sequence of a reverse primer for FAB1 contemplated for use according to the subject invention.

SEQ ID NO: 51 is the sequence of a forward primer for FAD2 contemplated for use according to the subject invention.

SEQ ID NO: 52 is the sequence of a reverse primer for FAD2 contemplated for use according to the subject invention.

SEQ ID NO: 53 is the sequence of a forward primer for FAD3 contemplated for use according to the subject invention.

SEQ ID NO: 54 is the sequence of a reverse primer for FAD3 contemplated for use according to the subject invention.

SEQ ID NO: 55 is the sequence of a forward primer for FAE1 contemplated for use according to the subject invention.

SEQ ID NO: 56 is the sequence of a reverse primer for FAE1 contemplated for use according to the subject invention.

SEQ ID NO: 57 is the sequence of a forward primer for fab1-c contemplated for use according to the subject invention.

SEQ ID NO: 58 is the sequence of a reverse primer for fab1-c contemplated for use according to the subject invention.

SEQ ID NO: 59 is the sequence of a forward primer for fad2-1 contemplated for use according to the subject invention.

SEQ ID NO: 60 is the sequence of a reverse primer for fad2-1 contemplated for use according to the subject invention.

SEQ ID NO: 61 is the sequence of a forward primer for fad3-a contemplated for use according to the subject invention.

SEQ ID NO: 62 is the sequence of a reverse primer for fad3-a contemplated for use according to the subject invention.

SEQ ID NO: 63 is the sequence of a forward primer for fae1-a contemplated for use according to the subject invention.

SEQ ID NO: 64 is the sequence of a reverse primer for fae1-a contemplated for use according to the subject invention.

SEQ ID NO: 65 is the sequence of a forward primer for fae1-c contemplated for use according to the subject invention.

SEQ ID NO: 66 is the sequence of a reverse primer for fae1-c contemplated for use according to the subject invention.

SEQ ID NO: 67 is a nucleotide sequence of Camelina sativa FAD2A contemplated for use according to the subject invention.

SEQ ID NO: 68 is a nucleotide sequence of Camelina sativa FAD2B contemplated for use according to the subject invention.

SEQ ID NO: 69 is a nucleotide sequence of Camelina sativa FAD2C contemplated for use according to the subject invention.

SEQ ID NO: 70 is a nucleotide sequence of Camelina sativa FAE1A contemplated for use according to the subject invention.

SEQ ID NO: 71 is a nucleotide sequence of Camelina sativa FAE1B contemplated for use according to the subject invention.

SEQ ID NO: 72 is a nucleotide sequence of Camelina sativa FAE1C contemplated for use according to the subject invention.

SEQ ID NO: 73 is a nucleotide sequence of Camelina saliva FAD3A contemplated for use according to the subject invention.

SEQ ID NO: 74 is a nucleotide sequence of Camelina sativa FAD3B contemplated for use according to the subject invention.

SEQ ID NO: 75 is a nucleotide sequence of Camelina sativa FAD3C contemplated for use according to the subject invention.

SEQ ID NO: 76 is a nucleotide sequence of Camelina sativa FAB1A contemplated for use according to the subject invention.

SEQ ID NO: 77 is a nucleotide sequence of Camelina sativa FAB1B contemplated for use according to the subject invention.

SEQ ID NO: 78 is a nucleotide sequence of Camelina sativa FAB1C contemplated for use according to the subject invention.

SEQ ID NO: 79 is a nucleotide sequence of the open reading frame of wild type Camelina saliva FAE1A contemplated for use according to the subject invention.

SEQ ID NO: 80 is a nucleotide sequence of the open reading frame of Camelina sativa FAE1A having a C442T mutation contemplated for use according to the subject invention.

SEQ ID NO: 81 is a nucleotide sequence of the open reading frame of wild type Camelina saliva FAE1C contemplated for use according to the subject invention.

SEQ ID NO: 82 is a nucleotide sequence of the open reading frame of Camelina saliva FAE1C having a C625T mutation contemplated for use according to the subject invention.

SEQ ID NO: 83 is a nucleotide sequence of the open reading frame of wild type Camelina saliva FAD2A contemplated for use according to the subject invention.

SEQ ID NO: 84 is a nucleotide sequence of the open reading frame of Camelina saliva FAD2A having a G449A mutation contemplated for use according to the subject invention.

SEQ ID NO: 85 is a nucleotide sequence of the open reading frame of wild type Camelina saliva FAD3A contemplated for use according to the subject invention.

SEQ ID NO: 86 is a nucleotide sequence of the open reading frame of Camelina sativa FAD3A having a G301A mutation contemplated for use according to the subject invention.

SEQ ID NO: 87 is the nucleotide sequence from position 415 to position 435 of Camelina saliva FAE1A or Camelina sativa FAE1B contemplated for use according to the subject invention.

SEQ ID NO: 88 is the nucleotide sequence from position 415 to position 435 of Camelina saliva FAE1A mutant contemplated for use according to the subject invention.

SEQ ID NO: 89 is the nucleotide sequence from position 415 to position 435 of Camelina saliva FAE1C contemplated for use according to the subject invention.

SEQ ID NO: 90 is the nucleotide sequence from position 418 to position 438 of Arabidopsis thaliana FAE1 contemplated for use according to the subject invention.

SEQ ID NO: 91 is the nucleotide sequence from position 613 to position 633 of Camelina sativa FAE1 A contemplated for use according to the subject invention.

SEQ ID NO: 92 is the nucleotide sequence from position 613 to position 633 of Camelina sativa FAE1B, and FAE1C or position 616 to position 636 of Arabidopsis thaliana FAE1 contemplated for use according to the subject invention.

SEQ ID NO: 93 is the nucleotide sequence from position 613 to position 633 of Camelina sativa FAE1C mutant contemplated for use according to the subject invention.

SEQ ID NO: 94 is the nucleotide sequence from position 439 to position 460 of Camelina sativa FAD2A, FAD2B, and FAD2C, or position 436 to position 457 of Arabidopsis thaliana FAD2 contemplated for use according to the subject invention.

SEQ ID NO: 95 is the nucleotide sequence from position 439 to position 460 of Camelina sativa FAD2A mutant contemplated for use according to the subject invention.

SEQ ID NO: 96 is the nucleotide sequence from position 291 to position 311 of Camelina sativa FAD3C, FAD3B, and FAD3A contemplated for use according to the subject invention.

SEQ ID NO: 97 is the nucleotide sequence from position 291 to position 311 of Camelina sativa FAD3A mutant contemplated for use according to the subject invention.

SEQ ID NO: 98 is the nucleotide sequence from position 288 to position 308 of Arabidopsis thaliana FAD3 contemplated for use according to the subject invention.

### DETAILED DESCRIPTION

Improving the oil quality of camelina is important for its increased use in specific applications, including biofuels and bio-based lubricants. In particular, the high PUFA content and the presence of VLCFA make the oil less useful as a biodiesel feedstock.

The subject invention provides materials and methods for generating improved populations of camelina (Camelina sativa) with altered seed oil composition. In specific embodiments, the subject invention provides methods for manipulating the fatty acid biosynthesis pathway in Camelina sativa, allowing for modifying the oil composition of this biofuel crop.

C. sativa is an allohexaploid whose oil composition can be influenced by one or more functional copies of FAE1, FAD3, FAB1 and FAD2. This allows highly specialized blends of oil to be produced from C. sativa with mutations in FAE1, FAD3, FAB1 and FAD2, thereby greatly increasing the utility of this crop.

Specifically, the present invention provides methods of altering and/or improving Camelina fatty acids composition by disrupting and/or altering one, two, or all three copies of one or more fatty acid synthesis genes in Camelina. In one embodiment, the methods comprise introducing mutations into one or more fatty acid synthesis genes. Such mutations include, for example, addition, deletion, and/or substitution of one or more nucleotides in the nucleic acid sequence of one or more fatty acid synthesis enzymes.

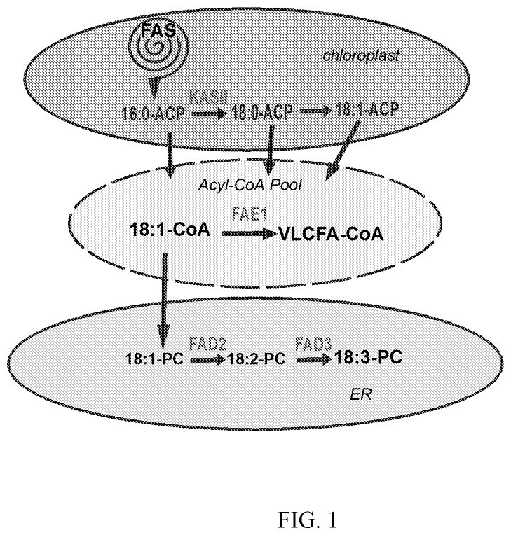

In a further embodiment, the present invention provides improved Camelina sativa fatty acid compositions. In preferred embodiments, the compositions have increased 18:1 (oleic acid) and decreased polyunsaturated fatty acids and long chain fatty acids. In accordance with the subject invention, one way by which 18:1 is increased and/or polyunsaturated fatty acids and long chain fatty acids are decreased is by lowering the activity of FAD2, FAD3 and of FAE1, and/or alter the activity of KASII, as indicated by the fatty acids synthesis pathway shown in FIG. 1.

In some embodiments, the methods comprise introducing mutations into one or more FAD2 genes (e.g., FAD2 A, FAD2 B, and FAD2 C), one or more FAD3 genes (e.g., FAD3 A, FAD3 B, and FAD3 C), and/or one or more FAE1 genes (e.g., FAE1 A, FAE1 B, and FAE1 C) of Camelina. In one embodiment, the methods further comprise introducing mutations into one or more FAB1 genes (e.g., FAB1 A, FAB1 B, and FAB1 C) of Camelina.

In one embodiment, the present invention provides an isolated nucleic acid sequence comprising a sequence selected from SEQ ID NOs: 67 to 98, and fragments and variants thereof, which encode a plant fatty acid synthesis gene.

In another embodiment, the present invention provides plant fatty acid synthesis proteins comprising a sequence selected from SEQ ID NOs: 1, 3-4, 30, 32-33, 35-37, 44-46, and fragments and variants thereof.

In some embodiments, the methods of altering and/or improving Camelina fatty acid compositions comprise utilizing one or more Camelina mutants as disclosed herein. In some embodiments, mutations in one or more copies of FAD2 genes, mutations in one or more copies of FAD3 genes, and/or mutations in one or more copies of FAE1 genes are integrated together to create mutant plants with double, triple, quadruple or more mutations. In a further embodiment, mutations in one or more copies of FAB1 genes may also be integrated into the mutant plants. In a specific embodiment, the mutant is a quadruple mutant, e.g., afae1c/fad2a/fae1a/fad3a quadruple mutant.

In one embodiment, the invention provides mutants in FAD2 A, FAD2 B, FAD2 C, FAD3 A, FAD3 B, FAD3 C, FAE1 A, FAE1 B, and/or FAE1 C genes of Camelina. The present invention also provides mutants in FAB1 A, FAB1 B, and/or FAB1 C genes of Camelina. In specific embodiments, the mutations are in the FAE1C, FAD2A, FAD3A, and/or FAE1A genes. In a preferred embodiment, the FAE1C gene of Camelina comprises a C625T mutation, the FAE1A gene of Camelina comprises a C422T mutation, the FAD2A gene of Camelina comprises a G449A mutation and/or the FAD3A gene of Camelina comprises a G301 A mutation.

In a specific embodiment, the mutant FAE1C comprises a sequence of SEQ ID NO: 82, or 93; the mutant FAD2A comprises a sequence of SEQ ID NO:84, or 95; the mutant FAD3A comprises a sequence of SEQ ID NO: 86, or 97; and the mutant FAE1A comprises a sequence of SEQ ID NO: 80, or 88.

In further embodiments, the present invention provides mutants in FAD2 A, FAD2 B, FAD2 C, FAD3 A, FAD3 B, FAD3 C, FAE1 A, FAE1 B, and/or FAE1 C proteins of Camelina. The present invention also provides mutants in FAB1 A, FAB1 B, and/or FAB1 C proteins of Camelina. In specific embodiments, the mutations are in the FAE1C, FAD2A, FAD3A, and/or FAE1A proteins. In a preferred embodiment, the FAE1C protein of Camelina comprise a R209* mutation, the FAE1A protein of Camelina comprises a P141L mutation, the FAD2A protein of Camelina comprises a G150E mutation, and/or the FAD3A protein of Camelina comprises a G101S mutation.

In one embodiment, the subject invention provides a mutant fatty acid synthesis enzyme comprising a polypeptide that comprises an amino acid sequence selected from SEQ ID NOs: 2, 5, 31, 38, 47 and fragments and variants thereof.

In one embodiment, the present invention provides a gene, or a chimeric gene, comprising the isolated nucleic acid sequence of any one of the polynucleotides described herein, operably linked to suitable regulatory sequences.

In one embodiment, the present invention provides a transformed host cell comprising the gene, or chimeric gene, as described herein. In certain embodiments, the host cell is selected from bacteria, yeasts, fungi, algae, animals, and plants.

In another embodiment, the present invention provides a plant, plant part, plant tissue, or plant cell comprising in its genome one or more genes as described herein. In a specific embodiment, the plant is a Camelina plant.

In some embodiments, the Camelina plant, plant part, plant tissue, or plant cell according to the subject invention has a reduced level of erucic acid (22:1) compared to a wild type Camelina plant, plant part, plant tissue, or plant cell. In some embodiments, said Camelina plant, plant part, plant tissue, or plant cell has less than 50%, less than 25%, less than 10%, less than 9%, less than 8%, less than 7%, less than 6%, less than 5%, less than 4%, less than 3%, less than 2%, less than 1%, or less than 0.1% erucic acid (22:1) compared to the wild type.

In certain embodiments, the Camelina plant, plant part, plant tissue, or plant cell has an increased level of 18:1 fatty acid, a reduced level of 20:1 fatty acid, a reduced level of 18:2 fatty acid and/or a reduced level of 18:3 fatty acid compared to the wild type.

In one embodiment, the present invention provides a plant seed obtained from the plants described herein, wherein the plants comprise in their genomes one or more genes as described herein, one or more genes with mutations as described herein, or the chimeric genes as described herein.

In one embodiment, the present invention provides Camelina oil, or oil composition, obtained from the seeds of a Camelina plant comprising the one or more genes described herein.

In one embodiment, the Camelina oil, or oil composition, obtained from a Camelina plant, plant part, plant tissue, or plant cell comprises at least about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, or about 50% oleic acid.

In one embodiment, the present invention provides meals made from Camelina plants comprising the one or more genes described herein, one or more genes with mutations as described herein, or one or more chimeric genes as described herein. In some embodiments, the meal is a byproduct of the extraction of the oil from said Camelina seeds. In further embodiments, the Camelina meal is included in the diet of an animal for about 0.1%, about 0.5%, about 1%, about 2%, about 3%, about 4%, about 5%, about 6%, about 7%, about 8%, about 9%, or about 10% of their feed on a weight or volume basis.

In one embodiment, the present invention provides methods of producing Camelina seed oil containing altered and/or increased levels of oleic acid (18:1), and/or altered or reduced levels of polyunsaturated fatty acids, and/or decreased very long chain fatty acids. Such methods comprise utilizing the Camelina plants comprising the genes as described herein, or Camelina plants with disrupted FAD2 (e.g., FAD2A, FAD2B, and FAD2C), FAD3 (e.g., FAD3A, FAD3B, and FAD3C), FAB1 (e.g., FAB1A, FAB1B, and FAB1C) and/or FAE1 (e.g., FAE1A, FAE1B, and FAE1C) genes as described herein.

### Definitions

The transitional term “comprising,” “comprises,” or “comprise” is inclusive or open-ended and does not exclude additional, unrecited elements or method steps. By contrast, the transitional phrase “consisting of” excludes any element, step, or ingredient not specified in the claim. The phrases “consisting” or “consists essentially of” indicate that the claim encompasses embodiments containing the specified materials or steps and those that do not materially affect the basic and novel characteristic(s) of the claim. Use of the term “comprising” contemplates other embodiments that “consist” or “consisting essentially of” the recited component(s).

As used herein, the term “plant” refers to any living organism belonging to the kingdom Plantae (i.e., any genus/species in the Plant Kingdom). This includes familiar organisms such as but not limited to trees, herbs, bushes, grasses, vines, ferns, mosses and green algae. The term refers to both monocotyledonous plants, also called monocots, and dicotyledonous plants, also called dicots.

Examples of particular plants include but are not limited to corn, potatoes, roses, apple trees, sunflowers, wheat, rice, bananas, tomatoes, opo, pumpkins, squash, lettuce, cabbage, oak trees, guzmania, geraniums, hibiscus, clematis, poinsettias, sugarcane, taro, duck weed, pine trees, Kentucky blue grass, zoysia, coconut trees, brassica leafy vegetables (e.g. broccoli, broccoli raab, Brussels sprouts, cabbage, Chinese cabbage (Bok Choy and Napa), cauliflower, cavalo, collards, kale, kohlrabi, mustard greens, rape greens, and other brassica leafy vegetable crops), bulb vegetables (e.g. garlic, leek, onion (dry bulb, green, and Welch), shallot, and other bulb vegetable crops), citrus fruits (e.g. grapefruit, lemon, lime, orange, tangerine, citrus hybrids, pummelo, and other citrus fruit crops), cucurbit vegetables (e.g. cucumber, citron melon, edible gourds, gherkin, muskmelons (including hybrids and/or cultivars of Cucumis melons), water-melon, cantaloupe, and other cucurbit vegetable crops), fruiting vegetables (including eggplant, ground cherry, pepino, pepper, tomato, tomatillo, and other fruiting vegetable crops), grape, leafy vegetables (e.g. romaine), root/tuber and corm vegetables (e.g. potato), and tree nuts (almond, pecan, pistachio, and walnut), berries (e.g., tomatoes, barberries, currants, elderberries, gooseberries, honeysuckles, mayapples, nannyberries, Oregon-grapes, see-buckthorns, hackberries, bearberries, lingonberries, strawberries, sea grapes, lackberries, cloudberries, loganberries, raspberries, salmonberries, thimbleberries, and wineberries), cereal crops (e.g., corn, rice, wheat, barley, sorghum, millets, oats, ryes, triticales, buckwheats, fonio, and quinoa), pome fruit (e.g., apples, pears), stone fruits (e.g., coffees, jujubes, mangos, olives, coconuts, oil palms, pistachios, almonds, apricots, cherries, damsons, nectarines, peaches and plums), vine (e.g., table grapes, wine grapes), fibber crops (e.g. hemp, cotton), ornamentals, and the like. For example, the plant is a species in the tribe of Camelineae, such as C. alyssum, C. anomala, C. grandiflora, C. hispida, C. laxa, C. microcarpa, C. microphylla, C. persistens, C. rumelica, C. sativa, C. Stiefelhagenii, or any hybrid thereof.

As used herein, the term “plant part” refers to any part of a plant including but not limited to the shoot, root, stem, seeds, stipules, leaves, petals, flowers, ovules, bracts, branches, petioles, internodes, bark, pubescence, tillers, rhizomes, fronds, blades, pollen, stamen, and the like. The two main parts of plants grown in some sort of media, such as soil, are often referred to as the “above-ground” part, also often referred to as the “shoots”, and the “below-ground” part, also often referred to as the “roots”.

The term “a” or “an” refers to one or more of that entity; for example, “a gene” refers to one or more genes or at least one gene. As such, the terms “a” (or “an”), “one or more” and “at least one” are used interchangeably herein. In addition, reference to “an element” by the indefinite article “a” or “an” does not exclude the possibility that more than one of the elements are present, unless the context clearly requires that there is one and only one of the elements.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 0 to 5%, or up to 1% of a given value.

As used herein, the term “chimeric protein” refers to a construct that links at least two heterologous proteins into a single macromolecule (fusion protein).

As used herein, the term “nucleic acid” refers to a polymeric form of nucleotides of any length, either ribonucleotides or deoxyribonucleotides, or analogs thereof. This term refers to the primary structure of the molecule, and thus includes double- and single-stranded DNA, as well as double- and single-stranded RNA. It also includes modified nucleic acids such as methylated and/or capped nucleic acids, nucleic acids containing modified bases, backbone modifications, and the like. The terms “nucleic acid” and “nucleotide sequence” are used interchangeably.

As used herein, the terms “polypeptide,” “peptide,” and “protein” are used interchangeably herein to refer to polymers of amino acids of any length. These terms also include proteins that are post-translationally modified through reactions that include glycosylation, acetylation and phosphorylation.

As used herein, the term “ortholog” is known in the art and refers to related sequences that share a common ancestor or family member and are determined based on the degree of sequence identity. The terms “homology”, “homologous”, “substantially similar” and “corresponding substantially” refer to nucleic acid fragments wherein changes in one or more nucleotide bases do not affect the ability of the nucleic acid fragment to produce a certain phenotype. These terms also refer to modifications of the nucleic acid fragments of the instant invention such as deletion or insertion of one or more nucleotides that do not substantially alter the functional properties of the resulting nucleic acid fragment relative to the initial, unmodified fragment. It is therefore understood, as those skilled in the art will appreciate, that the invention encompasses more than the specific exemplary sequences. The degree of sequence identity may vary, but in one embodiment, is at least 50% (when using standard sequence alignment programs known in the art), at least 60%, at least 65%, at least 70%, at least 75%, at least 80%, at least 85%, at least 90%, at least about 91%, at least about 92%, at least about 93%, at least about 94%, at least about 95%, at least about 96%, at least about 97%, at least about 98%, or at least 98.5%, or at least about 99%, or at least 99.5%, or at least 99.8%, or at least 99.9%. Homology can be determined using software programs readily available in the art, such as those discussed in Current Protocols in Molecular Biology (F. M. Ausubel et al., eds., 1987) Supplement 30, section 7.718, Table 7.71. Some alignment programs are MacVector (Oxford Molecular Ltd, Oxford, U.K.) and ALIGN Plus (Scientific and Educational Software, Pennsylvania). Other non-limiting alignment programs include Sequencher (Gene Codes, Ann Arbor, Mich.), AlignX, and Vector NTI (Invitrogen, Carlsbad, Calif.).

As used herein, the term “nucleotide change” or “nucleotide modification” refers to, e.g., nucleotide substitution, deletion, and/or insertion, as is well understood in the art. For example, mutations containing alterations that produce silent substitutions, additions, or deletions, but do not alter the properties or activities of the encoded protein or how the proteins are made.

As used herein, the term “protein modification” refers to, e.g., amino acid substitution, amino acid modification, deletion, and/or insertion, as is well understood in the art.

As used herein, the term “derived from” refers to the origin or source, and may include naturally occurring, recombinant, unpurified, or purified molecules. A nucleic acid or an amino acid derived from an origin or source may have all kinds of nucleotide changes or protein modification as defined elsewhere herein.

As used herein, the term “fragment” of a nucleic acid or polypeptide means a portion having the minimal size characteristics of such sequences, or any larger fragment of the full length molecule, up to and including the full length molecule. For example, a portion of a nucleic acid may be 12 nucleotides, 13 nucleotides, 14 nucleotides, 15 nucleotides, 16 nucleotides, 17 nucleotides, 18 nucleotides, 19 nucleotides, 20 nucleotides, 22 nucleotides, 24 nucleotides, 26 nucleotides, 28 nucleotides, 30 nucleotides, 32 nucleotides, 34 nucleotides, 36 nucleotides, 38 nucleotides, 40 nucleotides, 45 nucleotides, 50 nucleotides, 55 nucleotides, and so on, going up to the full length nucleic acid.

Similarly, a fragment of a polypeptide may be 4 amino acids, 5 amino acids, 6 amino acids, 7 amino acids, and so on, going up to the full length polypeptide. The length of the portion to be used will depend on the particular application. A fragment of a nucleic acid useful as hybridization probe may be as short as 12 nucleotides; in one embodiment, it is 20 nucleotides. A fragment of a polypeptide useful as an epitope may be as short as 4 amino acids. A portion of a polypeptide that performs the function of the full-length polypeptide would generally be longer than 4 amino acids.

As used herein, “sequence identity” or “identity” in the context of two nucleic acid or polypeptide sequences includes reference to the residues in the two sequences that are the same when aligned for maximum correspondence over a specified comparison window. When percentage of sequence identity is used in reference to proteins it is recognized that residue positions that are not identical often differ by conservative amino acid substitutions, where amino acid residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well-known to those of skill in the art. Typically this involves scoring a conservative substitution as a partial rather than a full mismatch, thereby increasing the percentage sequence similarity. Thus, for example, where an identical amino acid is given a score of 1 and a non-conservative substitution is given a score of zero, a conservative substitution is given a score between zero and 1. The scoring of conservative substitutions can be calculated, e.g., according to the algorithm of Meyers and Miller, Computer Applic. Biol. Sci., 4:11-17 (1988).

As used herein, the term “suppression” or “disruption” refers to reduced activity of proteins, and such reduced activity can be achieved by a variety of mechanisms including antisense, mutation knockout or RNAi. Antisense RNA will reduce the level of expressed protein resulting in reduced protein activity as compared to wild type activity levels. A mutation in the gene encoding a protein may reduce the level of expressed protein and/or interfere with the function of expressed protein to cause reduced protein activity.

As used herein, the terms “polynucleotide,” “polynucleotide sequence,” “nucleic acid sequence,” “nucleic acid fragment,” and “isolated nucleic acid fragment” encompass nucleotide sequences and the like. A polynucleotide may be a polymer of RNA or DNA that is single- or double-stranded, that optionally contains synthetic, non-natural or altered nucleotide bases. A polynucleotide in the form of a polymer of DNA may be comprised of one or more segments of cDNA, genomic DNA, synthetic DNA, or mixtures thereof. Nucleotides (usually found in their 5′-monophosphate form) are referred to by a single letter designation as follows: “A” for adenylate or deoxyadenylate (for RNA or DNA, respectively), “C” for cytidylate or deoxycytidylate, “G” for guanylate or deoxyguanylate, “U” for uridylate, “T” for deoxythymidylate, “R” for purines (A or G), “Y” for pyrimidines (C or T), “K” for G or T, “H” for A or C or T, “I” for inosine, and “N” for any nucleotide.

The term “primer” as used herein refers to an oligonucleotide that is capable of annealing to the amplification target allowing a DNA polymerase to attach, thereby serving as a point of initiation of DNA synthesis when placed under conditions in which synthesis of primer extension product is induced, i.e., in the presence of nucleotides and an agent for polymerization such as DNA polymerase and at a suitable temperature and pH. The (amplification) primer is preferably single stranded for maximum efficiency in amplification. Preferably, the primer is an oligodeoxyribonucleotide. The primer must be sufficiently long to prime the synthesis of extension products in the presence of the agent for polymerization. The exact lengths of the primers will depend on many factors, including temperature and composition (A/T and G/C content) of primer. A pair of bi-directional primers consists of one forward and one reverse primer as commonly used in the art of DNA amplification such as in PCR amplification.

As used herein, “coding sequence” refers to a DNA sequence that encodes a specific amino acid sequence. “Regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence.

As used herein, “regulatory sequences” may include, but are not limited to, promoters, translation leader sequences, introns, and polyadenylation recognition sequences.

As used herein, “promoter” refers to a DNA sequence capable of controlling the expression of a coding sequence or functional RNA. The promoter sequence consists of proximal and more distal upstream elements, the latter elements often referred to as enhancers. Accordingly, an “enhancer” is a DNA sequence that can stimulate promoter activity, and may be an innate element of the promoter or a heterologous element inserted to enhance the level or tissue-specificity of a promoter. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental conditions. It is further recognized that because in most cases the exact boundaries of regulatory sequences have not been completely defined, DNA fragments of some variation may have identical promoter activity. Promoters that cause a gene to be expressed in most cell types at most times are commonly referred to as “constitutive promoters”.

As used herein, the “3′ non-coding sequences” or “3′ UTR (untranslated region) sequence” refer to DNA sequences located downstream of a coding sequence and include polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor.

As used herein, the term “operably linked” refers to the association of nucleic acid sequences on a single nucleic acid fragment so that the function of one is regulated by the other. For example, a promoter is operably linked with a coding sequence when it is capable of regulating the expression of that coding sequence (i.e., that the coding sequence is under the transcriptional control of the promoter). Coding sequences can be operably linked to regulatory sequences in a sense or antisense orientation. In another example, the complementary RNA regions of the invention can be operably linked, either directly or indirectly, 5′ to the target mRNA, or 3′ to the target mRNA, or within the target mRNA, or a first complementary region is 5′ and its complement is 3′ to the target mRNA.

As used herein, the term “cross,” “crossing,” “cross pollination” or “cross-breeding” refer to the process by which the pollen of one flower on one plant is applied (artificially or naturally) to the ovule (stigma) of a flower on another plant.

As used herein, the term “gene” refers to any segment of DNA associated with a biological function. Thus, genes include, but are not limited to, coding sequences and/or the regulatory sequences required for their expression. Genes can also include nonexpressed DNA segments that, for example, form recognition sequences for other proteins. Genes can be obtained from a variety of sources, including cloning from a source of interest or synthesizing from known or predicted sequence information, and may include sequences designed to have desired parameters.

As used herein, the term “vector,” “plasmid,” or “construct” refers broadly to any plasmid or virus encoding an exogenous nucleic acid. The term should also be construed to include non-plasmid and non-viral compounds that facilitate transfer of nucleic acid into virions or cells, such as, for example, polylysine compounds and the like. The vector may be a viral vector that is suitable as a delivery vehicle for delivery of the nucleic acid, or mutant thereof, to a cell, or the vector may be a non-viral vector which is suitable for the same purpose. Examples of viral and non-viral vectors for delivery of DNA to cells and tissues are well known in the art and are described, for example, in Ma et al. (1997, Proc. Natl. Acad. Sci. U.S.A. 94:12744-12746). Examples of viral vectors include, but are not limited to, recombinant plant viruses. Non-limiting examples of plant viruses include ssDNA genomes viruses (e.g., family Geminiviridae), reverse transcribing viruses (e.g., families Caulimoviridae, Pseudoviridae, and Metaviridae), dsNRA viruses (e.g., families Reoviridae and Partitiviridae), (−) ssRNA viruses (e.g., families Rhabdoviridae and Bunyaviridae), (+) ssRNA viruses (e.g., families Bromoviridae, Closteroviridae, Comoviridae, Luteoviridae, Potyviridae, Sequiviridae and Tombusviridae) and viroids (e.g., families Pospiviroldae and Avsunviroidae. Examples of non-viral vectors include, but are not limited to, liposomes, polyamine derivatives of DNA, and the like.

Camelina sativa

Camelina is a genus of flowering plants belonging to the Brassicaceae family. Camelina saliva is a particular species of the genus Camelina that is important historically and is a source of oil that can be used in, for example, biofuels and lubricants.

The utility of a plant oil either for biodiesel or food depends on its fatty acid composition. Camelina has a fatty acid composition with high levels of both polyunsaturated fatty acids such as 18:2 and 18:3 (52-54%) as well as long chain fatty acids such as 20:1 (11-15%) and 22:1 (2-5%). For biofuel, the preferred fatty acid is 18:1 (oleic). Oleic has an advantageous balance of characteristics for cloud point vs. oxidative stability. Polyunsaturated fatty acids such as 18:2 and 18:3 have poor oxidative stability. The long chain fatty acids such as 20:1 and 22:1 contribute to out of range distillation temperatures in biodiesel. For biofuel utility it is therefore desirable to lower the level of polyunsaturated fatty acids and to lower the level of long chain fatty acids. The ultimate goal is to increase the percentage of 18:1 fatty acid. 18:1 is also considered a good fatty acid for food utility.

Camelina sativa is an allohexaploid plant and the C. sativa genome appears organized in three redundant and differentiated copies. The allohexaploid nature of the Camelina sativa genome has multiple implications. Its vigor and adaptability to marginal growth conditions may result at least in part from polyploidy. Polyploids are thought to be more adaptable to new or harsh environments, with the ability to expand into broader niches than either progenitor. Allohexaploidy might also affect any potential manipulations of the C. sativa genome, such as introgression of germplasm or induced mutations.

### Fatty Acids Synthesis in Plants

Fatty acid biosynthesis in plants takes place within the endoplasmic reticulum and plastids, the latter of which is an organelle widely thought to have originated from a photosynthetic bacterial symbiont.

During fatty acid biosynthesis, a repeated series of reactions incorporates acetyl moieties of acetyl-CoA into an acyl group 16 or 18 carbons long. The enzymes involved in this synthesis are acetyl-CoA carboxylase (ACCase), malonyl-CoA:ACP transacylase, 3-ketoacyl-ACP synthase I and III (KAS I and KAS III), 3-ketoacyl-ACP reductase, 2,3-trans-Enoyl-ACP reductase, 3-hydroxyacyl-ACP dehydratase (all referred as fatty acid synthase (FAS), except for ACCase). The name fatty acid synthase refers to a complex of several individual enzymes that catalyze the conversion of acetyl-CoA and malonyl-CoA to 16:0 and 18:0 fatty acids. Acyl-carrier protein (ACP), an essential protein cofactor, is generally considered a component of FAS.

The last three steps of the fatty acids synthesis cycle reduce a 3-ketoacyl substrate to form a fully saturated acyl chain. Each cycle of fatty acid synthesis adds two carbons to the acyl chain. Typically, fatty acid synthesis ends at 16:0 or 18:0, when one of several reactions stops the process. The most common reactions are hydrolysis of the acyl moiety from ACP by a thioesterase, transfer of the acyl moiety from ACP directly onto a glycerolipid by an acyl transferase, or double-bond formation on the acyl moiety by an acyl-ACP desaturase. The thioesterase reaction yields a sulfhydryl ACP.

Two principal types of acyl-ACP thioesterases occur in plants. For making storage lipids (triglycerides) in the ER, the FAT enzymes convert the fatty acid-ACP to a fatty acid-Co-A. The substrate for FAE1 is an R-CoA and it is an R-CoA that is added to various positions in the glycerol backbone during the Kennedy pathway portion of the synthesis of Triglycerides in the ER. The major class, designated FatA, is most active with 18:1 delta9-ACP. A second class designated FatB, typified by 16:0-ACP thioesterase, is most active with shorter-chain, saturated acyl-ACPs.

Three key enzymes also regulate the amount of 16:0, 18:0 and 18:1 fatty acids:acyl-acyl carrier protein thioesterase (also known as FATB), β-ketoacyl-acyl carrier protein (ACP) synthase II (KAS II) and Δ-9 desaturase. FATB hydrolyzes the fatty acyl group from acyl carrier protein (ACP) and thus determines the amount and type of fatty acid that is exported from the plastid. Suppression of FATB leads to a reduction in 16:0 and 18:0 (stearic acid) released to the cytoplasm. KAS II converts palmitoyl-ACP (16:0-ACP) to stearoyl-ACP (18:0 ACP), and thus the overexpression of KAS II leads to an increase in the amount of 16:0 being converted to 18:0. Δ-9 desaturase converts 18:0-ACP to oleoyl-ACP (18:1-ACP), and thus the overexpression of Δ-9 desaturase leads to an increase in the amount of 18:0 being converted to 18:1. Because the product of KAS II activity (18:0-ACP) is the substrate for Δ-9 desaturase, the overexpression of both KAS II and Δ-9 desaturase will lead to a further decrease in 16:0 and 18:0 and an increase in 18:1.

Unsaturated fatty acids are produced by desaturation of saturated lipids with the help of desaturases (FAD enzymes). Most fatty acid desaturases (FADs) in plants are integral membrane proteins, with the exception of a soluble, plastid-localized stearoyl-ACP desaturase.

Extensive surveys of the fatty acid composition of seed oils from different plant species have resulted in the identification of more than 200 naturally occurring fatty acids, which can broadly be classified into 18 structural classes, such as laballenic acid, stearolic acid, sterculynic acid, chaulmoogric acid, ricinoleic acid, vernolic acid, and furan-containing fatty acid. Less is known about the mechanisms responsible for the synthesis and accumulation of unusual fatty acids, or of their significance to the fitness of the plants that accumulate them. Unusual fatty acids occur almost exclusively in seed oils and may serve a defense function.

As used herein, the phrase “fatty acid synthesis genes” or “FAS gene” refers to any genes that are involved in synthesis of fatty acids, cuticle, and wax as described above. For example, such genes include, but are not limited to, malonyl-CoA:ACP transacylase, 3-ketoacyl-ACP synthase I and III (KAS I and KAS III), 3-ketoacyl-ACP reductase, 2,3-trans-Enoyl-ACP reductase, 3-hydroxyacyl-ACP dehydratase, acyl-ACP thioesterases, fatty acid desaturases (e.g., FAD2, FAD3), fatty acid elongases (e.g., FAE1), and hydroxylases.

Seed oil of Camelina sativa contains high levels (up to 45%) of omega-3 fatty acids, which is uncommon in vegetable sources. Over 50% of the fatty acids in cold pressed Camelina oil are polyunsaturated. The major components are alpha-linolenic acid—C18:3 (omega-3-fatty acid, approx 35-45%) and linoleic acid—C18:2 (omega-6 fatty acid, approx 15-20%). The composition of Camelina seed oil also comprises oleic acid (e.g., C18:1n9), eicosenoic acid (e.g., C20:1n9), palmitic acid (e.g., C16), erucic acid (e.g., C22:1n9), stearic acid (e.g., C18), eicosadienoic acid (e.g., C20:2n6), arachidic acid (e.g., C20), Mead's acid (e.g., C20:3n3), and/or octadecenoic acid (e.g., C18:1). The oil is also very rich in natural antioxidants, such as tocopherols, making this highly stable oil very resistant to oxidation and rancidity. It has 1-3% erucic acid. The vitamin E content of Camelina oil is approximately 110 mg/100 g.

In the endoplasmic reticulum, oleic acid (18:1) is converted to linoleic acid (18:2) by a delta-12-desaturase, fatty acid desaturase 2 (FAD2), which can further be converted to alpha-linolenic acid (C18:3) by fatty acid desaturase 3 (FAD3). Very long chain fatty acids (more than 18 carbons) are synthesized in the cytosol by extension of an 18 carbon fatty acid. The rate limiting step is thought to be the initial condensation step, catalyzed in the seed by fatty acid elongase 1 (FAE1). The saturated fatty acids palmitic acid (16:0) can be converted to stearic acid (18:0) by β-ketoacyl-acyl carrier protein (ACP) synthase II (KASII) encoded by the FATTYACID BIOSYNTHESIS1 (FAB1) gene. This enzyme initiates the two-carbon elongation of 16:0-ACP for formation of 18:0-ACP.

In specific embodiments, the present invention relates to increasing oleic acid (18:1) level, decreasing the level of long chain fatty acids, and/or improving the seed oil quality of Camelina. As used herein, the term “level” refers to the relative percentage of a component in a mixture.

FAD2, FAD3, FAB1 and FAE1 Genes of Camelina sativa Described herein are the full genomic sequences of three FAD2 genes, three FAD3 genes, three FAB1 genes, and three FAE1 genes from Camelina sativa. Camelina sativa FAD2 and FAE1 sequences have been deposited in Genbank at the NCBI [Genbank: GU929417-GU929422, SEQ ID NOs: 67 to 72, as listed below].

| GenBank | | |
|---|---|---|
| access # | Sequence Name | SEQ ID NO |
| GU929417 | Camelina sativa FAD2 A (upstream, coding and downstream | 67 |
| | genomic sequence) | |
| GU929418 | Camelina sativa FAD2 B (upstream, coding and downstream | 68 |
| | genomic sequence) | |
| GU929419 | Camelina sativa FAD2 C (upstream, coding and downstream | 69 |
| | genomic sequence) | |
| GU929420 | Camelina sativa FAE1 A [upstream gene (KCS17), intergenic | 70 |
| | region and coding) | |
| GU929421 | Camelina sativa FAE1 B (upstream gene (KCS17), intergenic | 71 |
| | region and coding) | |
| GU929422 | Camelina sativa FAE1 C [upstream gene (KCS17), intergenic | 72 |
| | region and coding) | |
| NC_025700.1 | Camelina sativa FAD3 A | 73 |
| NC_025691.1 | Camelina sativa FAD3 B | 74 |
| NC_025689.1 | Camelina sativa FAD3 C | 75 |
| NC_025693.1 | Camelina sativa FAB1 A | 76 |
| NC_025700.1 | Camelina sativa FAB1 B | 77 |
| NC_025691.1 | Camelina sativa FAB1 C | 78 |

In one embodiment, the present invention provides an isolated polynucleotide encoding a plant fatty acid synthesis enzyme, wherein the isolated polynucleotide comprises a nucleic acid sequence selected from the group consisting of SEQ ID NOs: 67 to 98, and nucleic acid sequences sharing at least 50%, at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identity to SEQ ID NOs: 67 to 98 and fragments thereof.

In one embodiment, the plant fatty acid synthesis enzyme is a plant fatty acid desaturase, a plant fatty acid elongase, or a plant beta-ketoacyl-ACP synthase. In a further embodiment, the plant fatty acid desaturase is, for example, FAD2 or FAD3. In a preferred embodiment, the plant fatty elongase is, for example, FAE1. In a specific embodiment, the plant beta-ketoacyl-ACP synthase is KASII.

In one embodiment, the present invention provides an isolated polynucleotide encoding a plant fatty acid desaturase, comprising a nucleic acid sequence of SEQ ID NO: 67, 68, 69, 73, 74, 75, 83-86, 94-98, and/or a nucleic acid sequence that shares at least 50%, at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identity to SEQ ID NO: 67, 68, 69, 73, 74, 75 83-86, 94-97, and/or 98. In a specific embodiment, the polynucleotide comprises one or more mutations according to the subject invention.

In another embodiment, the present invention provides an isolated polynucleotide encoding a fatty acid elongase, comprising a nucleic acid sequence of SEQ ID NO: 70, 71, 72, 79-82, 87-93 and/or a nucleic acid sequence that shares at least 50%, at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identity to SEQ ID NO: 70, 71, 72, 79-82, 87-92 and/or 93. In a specific embodiment, the polynucleotide comprises one or more mutations according to the subject invention.

In one embodiment, the present invention provides an isolated polynucleotide encoding a plant beta-ketoacyl-ACP synthase, comprising a nucleic acid sequence of SEQ ID NO: 76, 77, 78, and/or a nucleic acid sequence that shares at least 50%, at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identity to SEQ ID NO: 76, 77, and/or 78. In a specific embodiment, the polynucleotide comprises one or more mutations according to the subject invention.

In one embodiment, the present invention provides a gene comprising the isolated nucleic acid sequence of any one of the polynucleotides described herein, optionally operably linked to suitable regulatory sequences.

In one embodiment, the present invention provides a chimeric gene comprising the isolated nucleic acid sequence of any one of the polynucleotides described herein, optionally operably linked to suitable regulatory sequences.

In one embodiment, the present invention also provides a recombinant construct comprising the gene, or chimeric gene as described above. In one embodiment, the recombinant construct is a gene silencing construct, such as used in RNAi gene silencing.

The present invention also provides a transformed host cell comprising a gene as described above. In certain embodiments, said host cell is selected from the group consisting of bacteria, yeasts, filamentous fungi, algae, animals, and plants.

### Mutant FAS Genes of Camelina

The subject invention provides EMS mutants that have been created in Camelina sativa variety: CS2901, CS2362, CS2864 and CS1996. Analysis on lipid composition of these mutants using Gas Chromatography (GC) has been conducted. In addition, Tilling mutants have been identified in FAD2, FAD3, FAB1 and/or FAE1.

In some embodiments, mutations according to the subject invention are in one or more copies of FAD2 genes, one or more copies of FAD3 genes, one or more copies of FAB1 genes, and/or one or more copies of FAE1 genes. In one embodiment, the mutation is in the FAD2 A, FAD2 B, and/or FAD2 C genes. In one embodiment, the mutation is in the FAE1 A, FAE1 B, and/or FAE1 C genes. In one embodiment, the mutation is in the FAD3 A, FAD3 B, and/or FAD3 C genes. In one embodiment, the mutations is in the FAB1 A, FAB1 B, and/or FAB1 C genes.

In specific embodiments, the mutation are in the FAE1C, FAD2A, FAD3A, and/or FAE1A genes. In a preferred embodiment, FAE1C of Camelina sativa comprises a C625T mutation. In a preferred embodiment, FAE1A of Camelina sativa comprises a C422T mutation. In a preferred embodiment, FAD2A of Camelina sativa comprises a G449A mutation. In a preferred embodiment, FAD3A of Camelina sativa comprises a G301A mutation. In a preferred embodiment, FAB1C of Camelina sativa comprises a C1425T mutation.

In one embodiment, the present invention provides an isolated polynucleotide encoding a plant fatty acid desaturase, comprising a nucleic acid sequence that comprises one or more mutations selected from G301A and G449A mutations.

In another embodiment, the present invention provides an isolated polynucleotide encoding a fatty acid elongase, comprising a nucleic acid sequence that comprises one or more mutations selected from C625T and C422T mutations.

In one embodiment, the present invention provides an isolated polynucleotide encoding KASII, comprising a nucleic acid sequence that comprises a C1425T mutation.

In some embodiments, the present invention provides an isolated polynucleotide encoding a plant fatty acid desaturase, comprising a nucleic acid sequence that further comprises one or more mutations selected from G1516A, C1645T, C1746T, C1813T, G1844A, C1977T, G2015A, C2099T, G2155A, G1495A, G2272A, and G2138A. Preferably, the plant fatty acid desaturase is Camelina FAD2 A.

In some embodiments, the present invention provides an isolated polynucleotide encoding a plant fatty acid desaturase, comprising a nucleic acid sequence that further comprises one or more mutations selected from C207T, C213T, G785A, C476T, C176T, G462A, G498A, G779A, G737A, C812T, C882T, G410A, G675A, C459T, C528T, C987T, G416A, G650A, C656T, C203T, G582A, G372A, G322A, G374A, G926A, C490T, C940T, G148A and C284T. Preferably, the plant fatty acid desaturase is Camelina FAD2 B.

In some embodiments, the present invention provides an isolated polynucleotide encoding a plant fatty acid desaturase, comprising a nucleic acid sequence that further comprises one or more mutations selected from G1429A, C1501T, C1542T, C1576T, C1582T, G1607A, C1609T, G1619A, G1672A, G1717A, C1720T, C1741T, G1795A, G1796A, C1799T, G1808A, G1810A, C1857T, C1873T, G1880A, G1883A, G1890A, G1915A, G1948A, G1963A, C2029T, G2072A, G2080A, C2081T, C2084T, C2096T, C2110T, C2112T, G2117A, G2117A, G2140A, G2149A, C2188T, C2204T, G2255A, G2268A, C2285T, C2293T, C2315T, G2422A, G2443A, C1595T, and C2383T. Preferably, the plant fatty acid desaturase is Camelina FAD2 C.

In some embodiments, the present invention provides an isolated polynucleotide encoding a fatty acid elongase, comprising a nucleic acid sequence that further comprises one or more mutations selected from G621A, C695T, C714T, G798A, G801A, G805A, G810A, G810A, C817T, C820T, G821A, G867A, G877A, G997A, G997A, G1005A, C1042T, G1061A, G1065A, C1072T, C1083T, C1091T, G1120A, C1141T, C1167T, C1167T, G1254A, G1258A, C1272T, G1311A, G1354A, G1366A, G1387A, G1390A, G1401A, G1402A, G1402A, G1407A, G1416A, G1426A, G1426A, C1450T, G1463A and G1518A. Preferably, the plant fatty acid elongase is Camelina FAE1 A.

In some embodiments, the present invention provides an isolated polynucleotide encoding a fatty acid elongase, comprising a nucleic acid sequence that further comprises one or more mutations selected from C710T, C718T, G724A, C731T, C817T, G823A, G823A, C845T, G858A, G887A, C907T, C928T, C952T, G953A, G958A, G969A, G988A, C1019T, G1031A, G1042A, C1063T, C1082T, G1086A, G1109A, C1154T, C1229T, G1231A, C1271T, C1271T, G1275A, G1306A, C1310T, G1314A, C1310T, C1325T, G1337A, G1337A, G1343A, C1352T, C1384T, C1389T, G1412A, G1417A, G1427A, G1435A, G1441A, C1493T, and C1522T. Preferably, the plant fatty acid elongase is Camelina FAE1 B.

In some embodiments, the present invention provides an isolated polynucleotide encoding a fatty acid elongase, comprising a nucleic acid sequence that further comprises one or more mutations selected from A506T, A506T, A506T, A506T, A506T, C564T, C605T, G704A, C719T, G798A, C802T, C822T, C825T, G840A, G855A, G855A, C858T, C887T, C887T, C906T, C911 T, C911 T, G926A, G933A, G982A, C987T, G1010A, C1047T, G1067A, C1088T, G1115A, G1137A, C1154T, G1175A, C1251T, C1252T, G1255A, G1283A, G1287A, C1305T, C1305T, G1316A, C1353T, G1359A, C1400T, C1403T, G1406A, G1472A, C1486T, C1494T, and C1502T. Preferably, the plant fatty acid elongase is Camelina FAE1 C.

Variants and fragments of polynucleotides are also envisioned. The coding region may be altered by taking advantage of the degeneracy of the genetic code to alter the coding sequence such that, while the nucleotide sequence is substantially altered, it nevertheless encodes a protein having an amino acid sequence substantially similar to the disclosed FAD2, FAD3, FAB1 and FAE1 proteins. In one embodiment, variants may differ from the disclosed sequences by alteration of the coding region to fit the codon usage bias of the particular organism into which the molecule is to be introduced.

The sequences described herein facilitate the design of gene-specific primers and probes for FAD2, FAD3, FAB1 and FAE1. Primers are short nucleic acid molecules, for instance DNA oligonucleotides, usually 7 nucleotides or more in length, for example, that hybridize to contiguous complementary nucleotides or a sequence to be amplified. Longer DNA oligonucleotides may be about 15, 20, 25, 30 or 50 nucleotides or more in length. Primers can be annealed to a complementary target DNA strand by nucleic acid hybridization to form a hybrid between the primer and the target DNA strand, and then the primer extended along the target DNA strand by a DNA polymerase enzyme. Primer pairs can be used for amplification of a nucleic acid sequence, for example, by the PCR or other nucleic-acid amplification methods known in the art.

A probe comprises an identifiable, isolated nucleic acid that recognizes a target nucleic acid sequence. A probe typically includes a nucleic acid that is attached to an addressable location, a detectable label or other reporter molecule and that hybridizes to a target sequence. Typical labels include radioactive isotopes, enzyme substrates, co-factors, ligands, chemiluminescent or fluorescent agents, haptens, and enzymes.

Methods for preparing and using nucleic acid probes and primers are known in the art. One of ordinary skill in the art will appreciate that the specificity of a particular probe or primer increases with its length. Thus, in order to obtain greater specificity, probes and primers can be selected that comprise at least 20, 25, 30, 35, 40, 45, 50 or more consecutive nucleotides of a target nucleotide sequences.

FAD2, FAD3, FAB1 and FAE1 Proteins and Mutant Proteins of Camelina sativa

The present invention also provides polypeptides comprising at least a portion of the isolated protein selected from the group consisting of SEQ ID NOs: 1, 3-4, 30, 32-33, 35-37, 44-46, and variants thereof.

The present invention also provides an isolated amino acid sequence comprising a sequence selected from the group consisting of SEQ ID NOs: 1, 3-4, 30, 32-33, 35-37, 44-46, and fragments and variants thereof.

In some embodiments, the present invention provides isolated variant polypeptides comprising an amino acid sequence that shares at least 50%, at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% identity to SEQ ID NO: 1, 3-4, 30, 32-33, 35-37, 44-45 and/or 46.

The variants may contain alterations in the amino acid sequences of the constituent proteins. The term “variant” with respect to a polypeptide refers to an amino acid sequence that is altered by one or more amino acids with respect to a reference sequence. The variant can have “conservative” changes, or “nonconservative” changes, e.g., analogous minor variations can also include amino acid deletions or insertions, or both. The invention also encompasses fragments of proteins of FAD2, FAD3, FAB1 and FAE1.

Functional fragments and variants of a polypeptide include those fragments and variants that maintain one or more functions of the parent polypeptide. It is recognized that polypeptides can be considerably modified without materially altering one or more of the polypeptide's functions. Where an amino acid substitution is introduced, the mutation can be conservative and have no material impact on the relevant function(s) of a protein. Also, part of a polypeptide chain can be deleted without impairing or eliminating all of its functions. Further, insertions or additions can be made in the polypeptide chain for example, adding epitope tags, without impairing or eliminating its functions.

Other modifications that can be made without materially impairing one or more functions of a polypeptide can include, for example, in vivo or in vitro chemical and biochemical modifications or the incorporation of unusual amino acids. Such modifications include, but are not limited to, for example, acetylation, carboxylation, phosphorylation, glycosylation, ubiquination, labelling, e.g., with radionucleotides, and various enzymatic modifications, as will be readily appreciated by those well skilled in the art.

A variety of methods for labelling polypeptides, and labels useful for such purposes, are well known in the art, and include radioactive isotopes such as ³²P, ligands which bind to or are bound by labelled specific binding partners (e.g., antibodies), fluorophores, chemiluminescent agents, enzymes, and anti-ligands.

Conservative amino acid substitutions are those substitutions that, when made, least interfere with the properties of the original protein, that is, the structure and especially the function of the protein is conserved and not significantly changed by such substitutions. Conservative substitutions generally maintain (a) the structure of the polypeptide backbone in the area of the substitution, for example, as a sheet or helical conformation, (b) the charge or hydrophobicity of the molecule at the target site, or (c) the bulk of the side chain. The following table shows exemplary conservative amino acid substitutions.

| | Very Highly- | Highly Conserved | |
|---|---|---|---|
| Original | Conserved | Substitutions (from the | Conserved Substitutions |
| Residue | Substitutions | Blosum90 Matrix) | (from the Blosum65 Matrix) |
| Ala | Ser | Gly, Ser, Thr | Cys, Gly, Ser, Thr, Val |
| Arg | Lys | Gln, His, Lys | Asn, Gln, Glu, His, Lys |
| Asn | Gln; His | Asp, Gln, His, Lys, Ser, Thr | Arg, Asp, Gln, Glu, His, Lys, Ser, Thr |
| Asp | Glu | Asn, Glu | Asn, Gln, Glu, Ser |
| Cys | Ser | None | Ala |
| Gln | Asn | Arg, Asn, Glu, His, Lys, Met | Arg, Asn, Asp, Glu, His, Lys, Met, Ser |
| Glu | Asp | Asp, Gln, Lys | Arg, Asn, Asp, Gln, His, Lys, Ser |
| Gly | Pro | Ala | Ala, Ser |
| His | Asn; Gln | Arg, Asn, Gln, Tyr | Arg, Asn, Gln, Glu, Tyr |
| Ile | Leu; Val | Leu, Met, Val | Leu, Met, Phe, Val |
| Leu | Ile; Val | Ile, Met, Phe, Val | Ile, Met, Phe, Val |
| Lys | Arg; Gln; Glu | Arg, Asn, Gln, Glu | Arg, Asn, Gln, Glu, Ser, |
| Met | Leu; Ile | Gln, Ile, Leu, Val | Gln, Ile, Leu, Phe, Val |
| Phe | Met; Leu; Tyr | Leu, Trp, Tyr | Ile, Leu, Met, Trp, Tyr |
| Ser | Thr | Ala, Asn, Thr | Ala, Asn, Asp, Gln, Glu, Gly, Lys, Thr |
| Thr | Ser | Ala, Asn, Ser | Ala, Asn, Ser, Val |
| Trp | Tyr | Phe, Tyr | Phe, Tyr |
| Tyr | Trp; Phe | His, Phe, Trp | His, Phe, Trp |
| Val | Ile; Leu | Ile, Leu, Met | Ala, Ile, Leu, Met, Thr |

In some examples, variants can have no more than 1, 2, 3, 5, 10, 15, 20, 25, 30, 40, 50, or 100 conservative amino acid changes (such as very highly conserved or highly conserved amino acid substitutions). In other examples, one or several hydrophobic residues (such as Leu, Ile, Val, Met, Phe, or Trp) in a variant sequence can be replaced with a different hydrophobic residue (such as Leu, Ile, Val, Met, Phe, or Trp) to create a functional variant or the FAD2, FAD3, FAB1 and FAE1 proteins.

In one embodiment, the subject invention provides an isolated polypeptide of a plant fatty acid synthesis enzyme, wherein the polypeptide comprises an amino acid sequence selected from the group consisting of SEQ ID NOs: 1, 3-4, 30, 32-33, 35-37, and 44-46 with one or more mutations, wherein the plant fatty acid synthesis enzyme is a plant fatty acid desaturase, a plant fatty acid elongase, or a plant beta-ketoacyl-ACP synthase.

In one embodiment, the subject invention provides an isolated polypeptide of a plant fatty acid synthesis enzyme, wherein the polypeptide comprises an amino acid sequence that shares at least 50%, at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% identity to SEQ ID NOs: 1, 3-4, 30, 32-33, 35-37, 44, 45, and/or 46 wherein the amino acid sequence comprises one or more mutations according to the subject invention.

In one embodiment, the invention provides mutants that has been created in Camelina sativa variety: CS2901, CS2362, CS2864 and CS1996. In one embodiment, the mutants have been identified in FAD2, FAD3, FAB1 and/or FAE1 proteins.

In some embodiments, mutations according to the subject invention are in one or more FAD2 proteins, one or more FAD3 proteins, one or more FAB1 proteins, and/or one or more FAE1 proteins. In one embodiment, the mutation is in the FAD2 A, FAD2 B, and/or FAD2 C proteins. In one embodiment, the mutation is in the FAE1 A, FAE1 B, and/or FAE1 C proteins. In one embodiment, the mutation is in the FAD3 A, FAD3 B, and/or FAD3 C proteins. In one embodiment, the mutations is in the FAB1 A, FAB1 B, and/or FAB1 C proteins.

In specific embodiments, the mutation is in the FAE1C, FAD2A, FAD3A, and/or FAE1A proteins. In a preferred embodiment, FAE1C protein of Camelina sativa has a delition of R and/or is truncated compared to the wild type. In a preferred embodiment, FAE1A protein of Camelina saliva comprises a P141L mutation. In a preferred embodiment, FAD2A protein of Camelina sativa comprises a G150E mutation. In a preferred embodiment, FAD3A protein of Camelina sativa comprises a G101 S mutation. In a preferred embodiment, FAB1C protein of Camelina sativa comprises a P269L mutation.

In one embodiment, the isolated polypeptide comprises an amino acid sequence selected from the group consisting of SEQ ID NOs: 2, 5, 31, 38, 47 and fragments and variants derived from thereof. In some embodiments, the isolated polypeptide comprises an amino acid sequence that shares at least 50%, at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% identity to SEQ ID NOs: 2, 5, 31, 38, and/or 47 wherein the amino acid sequence comprises one or more mutations according to the subject invention.

In one embodiment, the present invention provides an isolated polypeptide comprising an amino acid sequence that comprises one or more of R209*, P141L, G150E, G101S, P269L mutation.

In some embodiments, the present invention provides an isolated polypeptide comprising an amino acid sequence that further comprises one or more mutations selected from G35R, L78F, H111=, P134S, R144H, V188=, G201D, S229F, G248R, E28K, E287K, and R242H. Preferably, the polypeptide is a Camelina FAD2 A protein, fragments or variant thereof.

In some embodiments, the present invention provides an isolated polypeptide comprising an amino acid sequence that further comprises one or more mutations selected from S53F, S55F, A246T, R143C, P43S, W138*, G150E, A244T, D230N, L255F, P278L, D121N, C209Y, S137F, P160L, T313M, P79S, V1231, G201S, P203S, R52C, G178E, C108Y, W91*, G109S, G293R, S147=, T297=, and T33=. Preferably, the polypeptide is a Camelina FAD2 B protein, fragments or variant thereof.

In some embodiments, the present invention provides an isolated polypeptide comprising an amino acid sequence that further comprises one or more mutations selected from E28K, R52C, S65=, L77F, P79S, W87*, P88S, W91*, G109S, G124S, L125F, L132F, G150R, G150E, S151F, R154K, D155N, G170=, P176S, G178E, R179H, M181I, G190R, G201S, G206R, L228F, R242H, A245T, A245V, A246V, A250V, L255F, L255=, G257E, G257E, A265T, V268I, P281S, S286F, G303E, K307=, T313M, H316Y, S323L, E359K, V3661, S83F, and Q346*. Preferably, the polypeptide is a Camelina FAD2 C protein, fragments or variant thereof.

In some embodiments, the present invention provides an isolated polypeptide comprising an amino acid sequence that further comprises one or more mutations selected from V551, L79=, L86F, V114M, A115T, C116Y, D118N, D118N, S120F, S121L, S121=, E137K, S140N, R180K, R180K, G183S, T195I, M201I, V2031, T2051, R209*, N211=, G221D, A228V, H237Y, H237Y, V2661, S267N, R272C, G285R, R299Q, G303E, R310Q, C311Y, G315R, G315E, G315E, D317N, G320S, G323E, G323E, T331I, G335=, and E354K. Preferably, the polypeptide is a Camelina FAE1 A protein, fragments or variant thereof.

In some embodiments, the present invention provides an isolated polypeptide comprising an amino acid sequence that further comprises one or more mutations selected from P76L, L79F, D81N, S83L, R112W, V114M, V114M, S121L, L125=, G135D, Q142*, P149S, R157C, R157H, E159K, Q162=, E169K, P179L, G183D, V187M, P194S, A200V, M201I, R209Q, A224V, T249I, E250K, S263F, S263F, M264I, G275R, A276V, A277=, A276V, S281F, G285E, G285E, R287Q, S290F, H301Y, T302=, R310Q, V312M, G315E, E318K, G320S, A337V, and P347S. Preferably, the polypeptide is a Camelina FAE1 B protein, fragments or variant thereof.

In some embodiments, the present invention provides an isolated polypeptide comprising an amino acid sequence that further comprises one or more mutations selected from T15S, T15S, T15S, T15S, T15S, S34F, L48F, D81N, L86F, R112Q, N113=, S120F, S121L, R126K, R131H, R131H, S132L, P142S, P142S, P148L, Q150*, Q150*, A155T, R157H, E173=, T1751, G183S, T195I, V2021, R209*, G218R, G225D, L231F, V2381, S263F, S263=, M264I, G274S, G275E, S281F, S281F, G285R, T297M, R299Q, Q313*, Q314*, G315R, A337T, N341=, T344M, and P347S. Preferably, the polypeptide is a Camelina FAE1 C protein, fragments or variant thereof.

Methods of Altering and/or Improving Camelina Seed Oil Composition

The present invention provides methods of altering and/or improving Camelina seed oil compositions. The present invention also provides methods of altering and/or improving the fatty acid composition of a Camelina plant cell, plant part, tissue culture or whole plant, wherein the method comprises disrupting and/or altering one, two, three or more copies of one or more Camelina fatty acids synthesis genes in the Camelina plant cell, plant part, tissue culture or whole plant.

As used herein, the term “altering” refers to any change of fatty acid composition in the seed oil, including, but not limited to, compound structure, distribution, relative ratio, and yield. The term “improving” refers to any change in seed oil composition that makes the seed oil composition better in one or more qualities for, for example, industrial or nutritional applications. Such improvement includes, but is not limited to, improved quality as meal, improved quality as raw material to produce biofuel, biodiesel, lubricant, more monounsaturated fatty acids and less polyunsaturated fatty acids, increased stability, lower cloud point, less NOx emissions, and reduced trans-fatty acids.

The quality of a biodiesel is greatly dependent upon its composition. Polyunsaturated fatty acid methyl esters (FAME) have been shown to disproportionately increase oxidation of biodiesel. The temperatures at which biodiesel forms crystals (the cloud point) and at which it can no longer be poured (the pour point) are also affected by composition: saturated FAMEs and long chain FAMEs greatly increase cloud point and pour point. Biodiesel higher in unsaturated FAMEs are, therefore, better in colder environments, but have a lower oxidative stability than biodiesel higher in saturated FAMEs. Polyunsaturated FAMEs have also been shown to result in increased NOx emissions while long chain fatty acids result in a biodiesel with too high of a distillation temperature by ASTM standards. A biodiesel high in 18:1 and low in polyunsaturated FAMEs and long chain FAMEs is thought to be the best combination, resulting in higher oxidative stability with a low enough cloud point and a high enough cetane number to meet biodiesel standards (ASTM D6751).

Meal is a significant byproduct of the extraction of oil from oilseeds for biofuel. To be able to take advantage of this byproduct as a protein supplement for, for example, livestock is important economically for biofuel producers. Camelina oil has about 1-4% erucic acid. High amounts of erucic acid have been linked to fatty deposits in the heart muscles of animals and glucosinolates lend an unpalatable taste and confer adverse effects on growth in animals. Thus, the development of lines with consistently <2% erucic acid is desirable. Camelina meal can be used in the diet of, for example, poultry, goat, cattle, turkeys, broiler chickens, feedlot beef cattle, swine, laying hens and dairy cattle.

FAE1 mutants with reduced very long chain fatty acids (VLCFA) such as 22:1 can be used to create Camelina varieties having oil, and thus meal, with <2% erucic acid. As used herein, the phrase “very long chain fatty acid” refers to a fatty acid with more than 18 carbons.

In another aspect, the present invention provides methods of producing Camelina seed oil containing altered and/or increased levels of oleic acid (18:1), and/or altered or reduced levels of polyunsaturated fatty acids, and/or decreased very long chain fatty acids. Such methods comprise utilizing the Camelina plants comprising the genes as described herein, or Camelina plants with disrupted FAD2, FAD3, FAB1 and/or FAE1 genes as described herein.

In one embodiment, the subject invention provides methods for increasing the monounsaturated fatty acids (e.g., oleic acids (18:1)) level and/or reducing the polyunsaturated fatty acids level in the seed oil, wherein the method comprises disrupting and/or altering one, two, three or more copies of one or more Camelina fatty acids synthesis genes. In some embodiments, one, two, or all three copies of Camelina FAD2, FAD3, FAB1 and/or FAE1 genes are disrupted.

In a specific embodiment, the mutations in FAD2A, FAE1C, FAD3A and/or FAE1A are integrated together to create mutant plants, plant parts, plant cells, plant tissue culture with double, triple, or quadruple mutations. In a preferred embodiment, the mutations in FAD2A, FAE1C, FAD3A and/or FAE1A are integrated together to create mutant plants, plant parts, plant cells, plant tissue culture with quadruple mutations.

In some embodiments, mutations described in the subject invention can be integrated into species closely related to Camelina sativa, such as other species in the Brassicaceae family, such as Brassica oleracea (cabbage, cauliflower, etc.), Brassica rapa (turnip, Chinese cabbage, etc.), Brassica napus (rapeseed, etc.), Raphanus sativus (common radish), Armoracia rusticana (horseradish), Matthiola (stock), and many others, with or without the help of marker-facilitated inter-cultivar gene transfer methods.

Non-limiting examples of improved seed oil are those having increased oleic acid, increased fatty acids of C18 or less (C≤18), decreased very long chain fatty acid (C>18), and/or decreased polyunsaturated fatty acids, in ratio and/or in absolute weight. As used herein, the term “C≤18” refers to a chemical compound having not more than 18 carbons; as used herein, the term C>18 refers to a chemical compound that has more than 18 carbons.

In other embodiments, amino acids in conserved domains or sites of Camelina FAD2, FAD3, FAB1 or FAE1 proteins can be compared to FAD2, FAD3, FAB1 or FAE1 orthologs in other species, e.g., closely related Brassicaceae species, or plant species with known FAD/FAB/FAE sequences, which do not contain mutations disclosed in the subject invention. Then, the FAD/FAB/FAE genes in these related species can be substituted or deleted to make mutants with reduced or abolished activity.

In one embodiment, the present invention provides methods of reducing the activity of FAD2, FAD3 and/or FAE1 protein in a Camelina plant cell, plant part, tissue culture or whole plant comprising transforming the plant cell, plant part, tissue culture or whole plant with a gene comprising FAD2, FAD3, and/or FAE1 gene encoding the polypeptide of the present invention, or functional variants thereof. In one embodiment, the gene is overexpressed.

In one embodiment, the oleic acid level in the seed oil produced from the Camelina plants of the present invention is increased as compared to the same plants known in the prior art (e.g., comparable wild type plant). For example, the level of oleic acid in the seed oil is increased by about 1%, about 2%, about 3%, about 4%, about 5%, about 6%, about 7%, about 8%, about 9%, about 10%, about 12%, about 14%, about 16%, about 18%, about 19%, about 20%, about 22%, about 24%, about 26%, about 28%, about 30%, about 32%, about 34%, about 36%, about 38%, about 40%, about 42%, about 44%, about 46%, about 48%, about 50%, about 52%, about 54%, about 56%, about 58%, about 59%, about 60%, about 62%, about 64%, about 66%, about 68%, about 70%, about 75%, about 80%, about 85%, about 90%, about 95%, about 100%, about 150%, about 200%, about 250%, about 300%, about 350%, about 400%, about 450%, or about 500%.

In another embodiment, the oleic acid yield in the seed oil produced per Camelina plant of the present invention is increased as compared to the same plants known in the prior art (e.g., comparable wild type plant). As used herein, the term “yield” refers to amount of one or more types of fatty acids produced per plant, or per acre. For example, the yield of oleic acid in the seed oil is increased by about 1%, about 2%, about 3%, about 4%, about 5%, about 6%, about 7%, about 8%, about 9%, about 10%, about 12%, about 14%, about 16%, about 18%, about 19%, about 20%, about 22%, about 24%, about 26%, about 28%, about 30%, about 32%, about 34%, about 36%, about 38%, about 40%, about 42%, about 44%, about 46%, about 48%, about 50%, about 52%, about 54%, about 56%, about 58%, about 59%, about 60%, about 62%, about 64%, about 66%, about 68%, about 70%, about 75%, about 80%, about 85%, about 90%, about 95%, about 100%, about 150%, about 200%, about 250%, about 300%, about 350%, about 400%, about 450%, or about 500%.

In another embodiment, the polyunsaturated fatty acid level and/or yield in the seed oil produced from the Camelina plants of the present invention is decreased as compared to the same plants known in the prior art (e.g., comparable wild type plant). For example, the level and/or yield of polyunsaturated fatty acid in the seed oil is decreased by about 1%, about 2%, about 3%, about 4%, about 5%, about 6%, about 7%, about 8%, about 9%, about 10%, about 12%, about 14%, about 16%, about 18%, about 19%, about 20%, about 22%, about 24%, about 26%, about 28%, about 30%, about 32%, about 34%, about 36%, about 38%, about 40%, about 42%, about 44%, about 46%, about 48%, about 50%, about 52%, about 54%, about 56%, about 58%, about 59%, about 60%, about 62%, about 64%, about 66%, about 68%, about 70%, about 75%, about 80%, about 85%, about 90%, about 95%, about 100%, about 150%, about 200%, about 250%, about 300%, about 350%, about 400%, about 450%, or about 500%.

In another embodiment, the very long chain fatty acid (C>18) level and/or yield in the seed oil produced from the Camelina plants of the present invention is decreased as compared to the same plants known in the prior art (e.g., comparable wild type plant). For example, the level and/or yield of very long chain fatty acid in the seed oil is decreased by about 1%, about 2%, about 3%, about 4%, about 5%, about 6%, about 7%, about 8%, about 9%, about 10%, about 12%, about 14%, about 16%, about 18%, about 19%, about 20%, about 22%, about 24%, about 26%, about 28%, about 30%, about 32%, about 34%, about 36%, about 38%, about 40%, about 42%, about 44%, about 46%, about 48%, about 50%, about 52%, about 54%, about 56%, about 58%, about 59%, about 60%, about 62%, about 64%, about 66%, about 68%, about 70%, about 75%, about 80%, about 85%, about 90%, about 95%, about 100%, about 150%, about 200%, about 250%, about 300%, about 350%, about 400%, about 450%, or about 500%.

In another embodiment, the fatty acids of C18 or less level and/or yield in the seed oil produced from the Camelina plants of the present invention is increased as compared to the same plants known in the prior art (e.g., comparable wild type plant). For example, the level and/or yield of fatty acids of C18 or less in the seed oil is increased by about 1%, about 2%, about 3%, about 4%, about 5%, about 6%, about 7%, about 8%, about 9%, about 10%, about 12%, about 14%, about 16%, about 18%, about 19%, about 20%, about 22%, about 24%, about 26%, about 28%, about 30%, about 32%, about 34%, about 36%, about 38%, about 40%, about 42%, about 44%, about 46%, about 48%, about 50%, about 52%, about 54%, about 56%, about 58%, about 59%, about 60%, about 62%, about 64%, about 66%, about 68%, about 70%, about 75%, about 80%, about 85%, about 90%, about 95%, about 100%, about 150%, about 200%, about 250%, about 300%, about 350%, or about 400%.

In addition, using the compositions and methods of the present invention, one skilled in the art will be able to combine disruption of FAD2, FAD3, FAC1 and/or FAE1 genes with other mutants and/or transgenes that can generally improve plant health, plant biomass, plant resistance to biotic and abiotic factors, plant yields, wherein the final preferred fatty acid production is increased. Such mutants and/or transgenes include, but are not limited to, cell cycle controlling genes, cell size controlling genes, cell division controlling genes, pathogen resistance genes, and genes controlling plant traits related to seed yield, which are well known to one skilled in the art (e.g., REV genes, KRP genes).

Methods of disrupting and/or altering a target gene have been known to one skilled in the art. These methods include, but are not limited to, mutagenesis (e.g., chemical mutagenesis, radiation mutagenesis, transposon mutagenesis, insertional mutagenesis, signature tagged mutagenesis, site-directed mutagenesis, and natural mutagenesis), knock-outs/knock-ins, antisense and RNA interference.

In one embodiment, the subject invention provides a plant cell, plant part, tissue culture or whole plant comprising at least two FAE1 genes each of which comprises at least one mutation that disrupts and/or alters the function of the gene.

In one embodiment, the subject invention provides a plant cell, plant part, tissue culture or whole plant comprising at least two FAE1 genes and at least one FAD2 gene each of which comprises at least one mutation that disrupts and/or alters the function of the gene.

In one embodiment, the subject invention provides a plant cell, plant part, tissue culture or whole plant comprising at least one FAE1 gene and at least one FAD2 gene each of which comprises at least one mutation that disrupts and/or alters the function of the gene.

In one embodiment, the subject invention provides a plant cell, plant part, tissue culture or whole plant comprising at least two, two, or three FAE1 genes, at least one, two, or three FAD2 genes, and at least one, two, or three FAD3 genes, each of which comprises at least one mutation that disrupts and/or alters the function of the gene.

In one embodiment, the plant cell, plant part, tissue culture or whole plant comprises at least three FAD2 genes and at least three FAE1 genes, each of which comprises at least one mutation that disrupts and/or alters the function of the gene.

In a further embodiment, the plant cell, plant part, tissue culture or whole plant further comprises at least one, two or three FAB1 genes, each of which comprises at least one mutation that disrupts and/or alters the function of the gene. The additional fatty acid gene is selected from the group consisting of a hydroxylase and a thioesterase.

The present invention also provides methods of breeding Camelina species producing altered levels of fatty acids in the seed oil and/or meal. In one embodiment, such methods comprise

i) making a cross between the Camelina mutants with mutations as described in the subject invention to a second Camelina species to make F1 plants;

ii) backcrossing said F1 plants to said second Camelina species;

iii) repeating backcrossing step until said mutations are integrated into the genome of said second Camelina species. Optionally, such method can be facilitated by molecular markers.

In one embodiment, the step of repeating the backcrossing step is to generate a near isogenic line, wherein the near isogenic line derived from the second Camelina plant with the integrated mutations has altered seed oil composition compared to that of the second Camelina plant without the integrated mutations; and wherein the mutations disrupt one or more copies of FAD2, one or more copies of FAD3, one or more copies of FAB1, and/or one or more copies of FAE1 genes.

In one embodiment, the method of breeding Camelina species producing altered levels of fatty acids in the seed oil and/or meal may further comprises backcrossing the F1 plant, or the plant produced by step iii) above to a third Camelina plant and optionally, repeating said backcrossing step to generate a near isogenic line, wherein the one or more mutations are integrated into the genome of the third Camelina plant.

In one embodiment, the near isogenic line derived from the third Camelina plant with the integrated mutations has altered seed oil composition compared to that of the second Camelina plant, or the third Camelina plant without the integrated mutations; wherein the mutations disrupt one or more copies of FAD2, one or more copies of FAD3, one or more copies of FAB1, and/or one or more copies of FAE1 genes.

The present invention provides methods of breeding species close to Camelina sativa, wherein said species produces altered levels of fatty acids in the seed oil and/or meal. In one embodiment, such methods comprise

i) making a cross between the Camelina mutants with mutations as described above to a species close to Camelina sativa to make F1 plants;

ii) backcrossing said F1 plants to said species that is close to Camelina saliva;

iii) repeating backcrossing step until said mutations are integrated into the genome of said species that is close to Camelina sativa. Special techniques (e.g., somatic hybridization) may be necessary in order to successfully transfer a gene from Camelina sativa to another species and/or genus, such as to B. oleracea. Optionally, such method can be facilitated by molecular markers.

### Plant Transformation

The polynucleotides of the present invention can be transformed into a Camelina plant, or other plants. A common method for the introduction of new genetic material into a plant genome involves the use of living cells of the bacterial pathogen Agrobacterium tumefaciens to inject a piece of DNA, called transfer or T-DNA, into individual plant cells (usually following wounding of the tissue) where it is targeted to the plant nucleus for chromosomal integration.

Agrobacterium-mediated plant transformation involves as a first step the placement of DNA fragments cloned on plasmids into living Agrobacterium cells, which are then subsequently used for transformation into individual plant cells. Agrobacterium-mediated plant transformation is thus an indirect plant transformation method. Methods of Agrobacterium-mediated plant transformation may also involve using vectors with no T-DNA, but with e.g., P-DNA in the transformation vector.

Other plant transformation methods include, but not are limited to, electroporation, “biolistic bombardment,” fibrous forms of metal or ceramic, silicon carbide and aluminium borate whiskers.

For efficient plant transformation, a selection method must be employed such that whole plants are regenerated from a single transformed cell and every cell of the transformed plant carries the DNA of interest. These methods can employ a positive selection, whereby a foreign gene is supplied to a plant cell that allows it to utilize a substrate present in the medium that it otherwise could not use, such as mannose or xylose. A negative selection using one or more selective agents can be more efficient. The selective agents may be, for example, herbicides and/or antibiotics that either kill or inhibit the growth of nontransformed plant cells and reducing the possibility of chimeras.

Either a gamete-specific promoter, a constitutive promoter (such as the CaMV or Nos promoter), an organ-specific promoter (such as the E8 promoter from tomato), or an inducible promoter is typically ligated to the protein or antisense encoding region using standard techniques known in the art. The expression unit may be further optimized by employing supplemental elements such as transcription terminators and/or enhancer elements.

For example, the 5′ introns of FAD2 gene in sesame have been demonstrated to increase and/or regulate expression of certain genes (Kim et al. 2006. Mol Genet Genomics 276(4): 351-68). Thus, the 5′ intron sequences of the FAD2 genes of the present invention can be used to increase expression of either a FAD2 or a non-FAD2 gene. The expression cassette can comprise, for example, a seed-specific promoter (e.g. the phaseolin promoter (U.S. Pat. No. 5,504,200). The term “seed-specific promoter”, means that a gene expressed under the control of the promoter is predominantly expressed in plant seeds with no or no substantial expression, typically less than 10% of the overall expression level, in other plant tissues. Seed specific promoters have been well known in the art.

Thus, for expression in plants, the expression units will typically contain, in addition to the protein sequence, a plant promoter region, a transcription initiation site and a transcription termination sequence. Unique restriction enzyme sites at the 5′ and 3′ ends of the expression unit are typically included to allow for easy insertion into a pre-existing vector.

In the present invention, “overexpression promoter” means a promoter capable of causing strong expression (large amount expression) of a gene that has been ligated thereto in host plant cells. The overexpression promoter of the present invention may be either an inducible promoter or a constitutive promoter. A promoter is a DNA comprising an expression control region generally located on the 5′ upstream of a structural gene or a modified sequence thereof. In the present invention, any promoters appropriate for foreign gene expression in plant cells can be used as overexpression promoters.

Non-limiting examples of such overexpression promoters to be used in the present invention include, but are not limited to, a cauliflower mosaic virus (CaMV) 35S promoter, a rice actin promoter, a modified 35S promoter, or an embryo-specific promoter. As used herein an “embryo-specific promoter” refers to a promoter of an embryo-specific gene. An embryo-specific gene is preferentially expressed during embryo development in a plant. For purposes of the present disclosure, embryo development begins with the first cell divisions in the zygote and continues through the late phase of embryo development (characterized by maturation, desiccation, dormancy), and ends with the production of a mature and desiccated seed. Embryo-specific genes can be further classified as “early phase-specific” and “late phase-specific”. Early phase-specific genes are those expressed in embryos up to the end of embryo morphogenesis. Late phase-specific genes are those expressed from maturation through to production of a mature and desiccated seed. An early phase-specific promoter is a promoter that initiates expression of a protein prior to day 7 after pollination in Arabidopsis or an equivalent stage in another plant species.

Non-limiting examples of promoter sequences that can be used in the present invention include a promoter for the amino acid permease gene (AAP1), a promoter for the oleate 12-hydroxylase:desaturase gene (e.g., the promoter designated LFAH 12 from Lesquerellafendleri), a promoter for the 2S2 albumin gene (e.g., the 2S2 promoter from Arabidopsis thaliana), a fatty acid elongase gene promoter (FAE1) (e.g., the FAE1 promoter from Arabidopsis thaliana), and the leafy cotyledon gene promoter (LEC2) (e.g., the LEC2 promoter from Arabidopsis thaliana). Other early embryo-specific promoters of interest include, but are not limited to, seedstick, Fbp7 and Fbpl 1, Banyuls, agl-15 and agl-18, Phel, Perl, embl75, LI1, Lec1, Fusca3, tt12, tt16, A-RZf, TtGl, TtI, TT8, Gea-8 (carrot), Knox (rice), Oleosin, ABI3, and the like.

In addition to a promoter sequence, the expression cassette can also contain a transcription termination region downstream of the structural gene to provide for efficient termination. The termination region may be obtained from the same gene as the promoter sequence or may be obtained from different genes. If the mRNA encoded by the structural gene is to be efficiently processed, DNA sequences which direct polyadenylation of the RNA are also commonly added to the vector construct. The vector will typically contain a selectable marker gene expression unit by which transformed plant cells can be identified in culture. Usually, the marker gene will encode resistance to an antibiotic, such as G418, hygromycin, bleomycin, kanamycin, or gentamicin or to an herbicide, such as glyphosate (Round-Up) or glufosinate (BASTA) or atrazine.

Replication sequences, of bacterial or viral origin, are generally also included to allow the vector to be cloned in a bacterial or phage host; preferably a broad host range for prokaryotic origin of replication is included. A selectable marker for bacteria may also be included to allow selection of bacterial cells bearing the desired construct. Suitable prokaryotic selectable markers include resistance to antibiotics such as ampicillin, kanamycin or tetracycline. Other DNA sequences encoding additional functions may also be present in the vector, as is known in the art. For instance, in the case of Agrobacterium transformations, T-DNA sequences will also be included for subsequent transfer to plant chromosomes.

The expression vectors of the present invention will preferably include at least one selectable marker. Such markers include, for example, dihydrofolate reductase, G418 or neomycin resistance for eukaryotic cell culture and tetracycline, kanamycin or ampicillin resistance genes for culturing in E. coli and other bacteria. Vectors that can be used with the invention include vectors for use in bacteria, which comprise pQE70, pQE60 and pQE-9, pBluescript vectors, Phagescript vectors, pNH8A, pNH16a, pNH18A, pNH46A, ptrc99a, pKK223-3, pKK233-3, pDR540, pRIT5. Among preferred eukaryotic vectors are pFastBac1 pWINEO, pSV2CAT, pOG44, pXTI and pSG, pSVK3, pBPV, pMSG, and pSVL. Other suitable vectors will be readily apparent to the skilled artisan.

Recombinant DNA techniques allow plant researchers to identify and clone specific genes for desirable traits, such as improved fatty acid composition, and to introduce these genes into already useful varieties of plants. Once the foreign genes have been introduced into a plant, that plant can then be used in conventional plant breeding schemes (e.g., pedigree breeding, single-seed-descent breeding schemes, reciprocal recurrent selection) to produce progeny which also contain the gene of interest.

Genes can be introduced in a site directed fashion using homologous recombination.

Homologous recombination permits site-specific modifications in endogenous genes and thus inherited or acquired mutations may be corrected, and/or novel alterations may be engineered into the genome.

### Breeding Methods

Classic breeding methods can be included in the present invention to introduce one or more mutations of the present invention into other Camelina varieties, or other close-related species of the Brassicaceae family that are compatible to be crossed with Camelina. In one embodiment, the mutations are on the FAD2 A, FAD2 B, and/or FAD2 C genes. In one embodiment, the mutations are on the FAE1 A, FAE1 B, and/or FAE1 C genes. In one embodiment, the mutations are on the FAD3 A, FAD3 B, and/or FAD3 C genes. In one embodiment, the mutations are on the FAB1 A, FAB1 B, and/or FAB1 C genes. In one embodiment, the mutations are on any one or more FAD2 gene, FAD3 gene, FAB1 gene and/or FAE1 gene.

Open-Pollinated Populations. The improvement of open-pollinated populations of such crops as rye, many maizes and sugar beets, herbage grasses, legumes such as alfalfa and clover, and tropical tree crops such as cacao, coconuts, oil palm and some rubber, depends essentially upon changing gene-frequencies towards fixation of favorable alleles while maintaining a high (but far from maximal) degree of heterozygosity. Uniformity in such populations is impossible and trueness-to-type in an open-pollinated variety is a statistical feature of the population as a whole, not a characteristic of individual plants. Thus, the heterogeneity of open-pollinated populations contrasts with the homogeneity (or virtually so) of inbred lines, clones and hybrids.

Population improvement methods fall naturally into two groups, those based on purely phenotypic selection, normally called mass selection, and those based on selection with progeny testing. Interpopulation improvement utilizes the concept of open breeding populations; allowing genes to flow from one population to another. Plants in one population (cultivar, strain, ecotype, or any germplasm source) are crossed either naturally (e.g., by wind) or by hand or by bees (commonly Apis mellifera L. or Megachile rotundata F.) with plants from other populations. Selection is applied to improve one (or sometimes both) population(s) by isolating plants with desirable traits from both sources.

There are several primary methods of open-pollinated population improvement. First, there is the situation in which a population is changed en masse by a chosen selection procedure. The outcome is an improved population that is indefinitely propagable by random-mating within itself in isolation. Second, the synthetic variety attains the same end result as population improvement but is not itself propagable as such; it has to be reconstructed from parental lines or clones. Third, a method used in plant species that are largely self pollinated in nature, such as soybeans, wheat, rice, safflower, camelina and others is pedigree selection. In this situation, crosses are made and individual plants and lines from individual plants are selected for desired traits. These lines are advanced as genetically homogeneous varieties. Since the individuals are largely self pollinated, these lines are analogous to an inbred line with favorable agronomic characteristics.

Mass Selection. In mass selection, desirable individual plants are chosen, harvested, and the seed composited without progeny testing to produce the following generation. Since selection is based on the maternal parent only, and there is no control over pollination, mass selection amounts to a form of random mating with selection. As stated above, the purpose of mass selection is to increase the proportion of superior genotypes in the population.

Synthetics. A synthetic variety is produced by crossing inter se a number of genotypes selected for good combining ability in all possible hybrid combinations, with subsequent maintenance of the variety by open pollination. Whether parents are (more or less inbred) seed-propagated lines, as in some sugar beet and beans (Vicia) or clones, as in herbage grasses, clovers and alfalfa, makes no difference in principle. Parents are selected on general combining ability, sometimes by test crosses or topcrosses, more generally by polycrosses. Parental seed lines may be deliberately inbred (e.g. by selfing or sib crossing). However, even if the parents are not deliberately inbred, selection within lines during line maintenance will ensure that some inbreeding occurs. Clonal parents will, of course, remain unchanged and highly heterozygous.

Whether a synthetic can go straight from the parental seed production plot to the farmer or must first undergo one or two cycles of multiplication depends on seed production and the scale of demand for seed. In practice, grasses and clovers are generally multiplied once or twice and are thus considerably removed from the original synthetic.

While mass selection is sometimes used, progeny testing is generally preferred for polycrosses, because of their operational simplicity and obvious relevance to the objective, namely exploitation of general combining ability in a synthetic.

The number of parental lines or clones that enter a synthetic vary widely. In practice, numbers of parental lines range from 10 to several hundred, with 100-200 being the average. Broad based synthetics formed from 100 or more clones would be expected to be more stable during seed multiplication than narrow based synthetics.

Pedigreed varieties. A pedigreed variety is a superior genotype developed from selection of individual plants out of a segregating population followed by propagation and seed increase of self pollinated offspring and careful testing of the genotype over several generations. This is an open pollinated method that works well with naturally self pollinating species. This method can be used in combination with mass selection in variety development. Variations in pedigree and mass selection in combination are the most common methods for generating varieties in self pollinated crops.

Hybrids. A hybrid is an individual plant resulting from a cross between parents of differing genotypes. Commercial hybrids are now used extensively in many crops, including corn (maize), sorghum, sugarbeet, sunflower and broccoli. Hybrids can be formed in a number of different ways, including by crossing two parents directly (single cross hybrids), by crossing a single cross hybrid with another parent (three-way or triple cross hybrids), or by crossing two different hybrids (four-way or double cross hybrids).

The present invention is further illustrated by the following examples that should not be construed as limiting. The contents of all references, patents, and published patent applications cited throughout this application, as well as the Figures, are incorporated herein by reference in their entirety for all purposes.

### EXAMPLES

### Methods and Materials

### Plant Growth and Characterization

Camelina sativa (cultivar Ames 1043) seed lines were planted into moist soil media (soil, perlite, vermiculture, and fertilizer) and grown at 21° C. under a 16 h day/8 h night light cycle in greenhouses or growth chambers. Mature seeds were harvested, dried thoroughly, and stored at room temperature until further analyzed. Lines were crossed by emasculating green flower buds of the pollen recipient and then applying pollen from anthers removed from the donor plant.

### Screening of the Mutant Population

Fatty acid methyl esters (FAMEs) were prepared by transesterification with trimethylsulphonium hydroxide (TMSH). Single transgenic seeds were directly crushed in 50 μL of TMSH in glass GC vials. Heptane (450 μL) was added to each vial. After room temperature incubation with agitation for 30 min, FAMEs were quantified on an Agilent Technologies 7890A gas chromatograph fitted with a flame ionization detector and a 30 m length×0.25 mm inner diameter HP-INNOWax column (Agilent, CA, USA) using helium as the carrier gas. The oven temperature was set to start at 90° C. with a 1 min hold and then an increase of 30° C./min until it reached 235° C., for 5 min.

### Fatty Acid Quantification of Camelina Seed

FAMEs were generated from ˜10 mg of dry camelina seeds using a previously established acid catalyzed method, with triheptadecanoin (Nu-Check Prep, MN) added as an internal standard. The organic phase was transferred to autosampler vials and FAMEs quantified using gas chromatography as described above.

### FAD2 and FAE1 Sequence Alignments

Translated amino acid FAD2 and FAE1 sequences were aligned with AlignX (Invitrogen), with a gap opening penalty of 15, a gap extension penalty of 6.66, and a gap separation penalty range of 8. Alignments were imported into Boxshade (EMBnet) to highlight the conserved residues.

### Identification and Isolation of Mutant Alleles

Genomic DNA was extracted from young leaf tissue using the DNeasy Plant Mini kit (QIAGEN Sciences, MD, USA). Gene candidates were amplified with PCR using Phusion High-Fidelity DNA polymerase (Fisher Scientific) as per the manufacturer's protocol and primer pairs specific for all three gene homeologs (Table 1).

TABLE 1
Primers to subclone the FAB1C, FAD2A, FAD3A,
FAE1A, and FAE1C genes into pCRBlunt or
pGEM-T easy vectors

| | Forward primer | Reverse primer |
|---|---|---|
| Gene | [SEQ ID NO] | [SEQ ID NO] |
| FAB | 5′-GAGCGGCCGCA | 5′-ATGCGGCCGCG |
| 1 | TCACAGATCGATT | AACAATGTATGTAA |
| | CTCTCTTAA-3′ | TGTACC-3′ [50] |
| | [49] | |
| FAD | 5′-ATGGGTGCAGG | 5′-CCTCATAACTT |
| 2 | TGGAAGAATG-3′ | ATTGTTGTACC-3′ |
| | [51] | [52] |
| FAD | 5′-ATGGTTGTTGC | 5′-TTAATTGATTT |
| 3 | TATGGACAAACG- | TAGACTTGTCAGAA |
| | 3′ [53] | GCG-3′ [54] |
| FAE | 5′-ATGACGTCCGT | 5′-TTAGGACCGAC |
| 1 | TAACGCAAAG-3′ | CGTTTTTGAC-3′ |
| | [55] | [56] |

PCR products were ligated into pCRBlunt or pGEM-T easy vectors (Invitrogen, CA, USA) and transformed into DH5a chemical competent Escherichia coli cells. Plasmid DNA was isolated from individual colonies and sequenced to identify mutant alleles. Because FAE1A, FAE1C and FAD2A lack introns, these cloned genomic sequences were also used for subsequent expression experiments. To isolate cDNA for FAD3A, which does contain introns, RNA was extracted from developing wild type and CS2864 line seeds using a cetyltrimethylammonium bromide (CTAB) method (Bekesiova et al., 1999). Briefly, 1.4 mls of RNA extraction buffer, which also included 0.5 g/L spermidine, was added to the ground tissue and incubated for 30 min at 65° C. with occasional mixing. 8M LiCl was used to precipitate the RNA. 450 μl Buffer RLT from the RNeasy Plant Mini kit (QIAGEN Sciences, MD, USA) was added to the RNA and steps 4-11 of the kit were followed. 1p g RNA was treated with DNase and cDNA was synthesized using the RevertAid RT Reverse Transcription kit (Thermofisher Scientific, MA, USA). FAD3A homeologs were then amplified, cloned into pCRBlunt and then identified by sequencing.

### Yeast Expression

The open reading frames for wild type and mutant alleles of FAE1A, FAE1C and FAD2A were ligated into the EcoRI site of the yeast expression vector pYES2 (Invitrogen, CA, USA) under the control of the inducible GAL1 promoter. Wild type FAD3A or the mutant fad3a allele were ligated into the BamHI/XhoI sites of the yeast expression vector pESC-URA under control of the GAL1 promoter; wild type FAD2A was cloned downstream of the GAL10 promoter to enable expression of both genes. The pYES2 and pESC-URA plasmids were transformed into Saccharomyces cerevisiae yeast strain BY4741 using the Frozen-EZ Yeast Transformation II Kit (Zymo Research, CA, USA) and selected on minimal agar plates lacking uracil. Single colonies were inoculated into synthetic complete minimal medium lacking uracil (SC-Ura) which contained 2% raffinose as the exclusive carbon source and grown at 28° C. for 48 hours with shaking at 250 rpm. Cells were harvested, washed with sterile water and diluted to 0.2 OD₆₀₀ in SC-Ura induction medium containing 2% galactose. After a further 48 h growth at 28° C. for the FAE1 vectors and 96 hour growth at 20° C. for the FAD2 and FAD3 vectors, the yeast cells were pelleted and washed three times with water to remove media or other metabolites, cells were dried in a freeze dryer and stored at −80° C. until lipids were extracted and analyzed. Fatty acid methyl esters (FAMEs) were generated by resuspending dried yeast cells in 1.5 ml of 2.5% sulfuric acid (H₂SO₄, v/v) in methanol and heated for 30 min at 90° C. in 13×100 mm glass screw-capped tubes. Following cooling, 1.5 ml of water and 2 ml of heptane were added to the tubes and mixed. The organic phase was transferred to autosampler vials and analyzed on an Agilent Technologies 7890A gas chromatograph as for the camelina FAMEs.

### Camelina Mutant Complementation

pCRBlunt clones containing cDNAs for the FAE1A, FAE1C, FAD2A or FAD3A wild type alleles were amplified by PCR, digested with EcoRI/XhoI, and ligated under control of the CaMV35S promoter into the corresponding sites in the binary plant vector pBin35SRed, a variant of the previously described pBinGlyRed3 (Nguyen et al., 2015; Nguyen et al., 2013) with the glycinin-1 promoter replaced with a CaMV35 promoter. This vector also contains the DsRed (Discosoma coral) gene under control of the cassava mosaic virus 35S promoter as the seed selection marker. The resulting vectors were transformed into Agrobacterium tumefaciens strain C58CI. Transgenic camelina lines were generated using an Agrobacterium mediated vacuum method and DsRed-positive seeds were identified using a green LED flashlight with a red camera filter lens (Lu and Kang, 2008). FAMEs were prepared by transesterification with TMSH and quantified on an Agilent Technologies 7890A gas chromatograph as described herein.

### Genotyping Using CAPS/dCAPS

CAPS (Cleaved Amplified Polymorphic Sequences) or dCAPS (Derived Cleaved Amplified Polymorphic Sequences) markers were designed to distinguish the different mutant alleles from the wild type genes. The dCAPS assays were designed using the dCAPS Finder 2.0 software. Primer sequences and restriction enzymes used for each assay are listed in Table 2.

TABLE 2
Primers to genotype the fab1-c, fad2-a,
fad3-a, fae1-a, and fae1-c alleles.

| | | | | | | An- | |
|---|---|---|---|---|---|---|---|
| | | | For- | Re- | | neal- | Elec- |
| | | | ward | verse | Re- | ing | tropho- |
| | | | Primer | Primer | stric- | Tm | resis |
| Meth- | | Al- | [SEQ | [SEQ | tion | (° | con- |
| od | LINE | lele | ID NO] | ID NO] | Site | C.) | ditions |
| dCAP | CS19 | fab1- | 5′- | 5′- | BgHI | 52 | 2.5% |
| S | 96 | c | ATCTGA | ACATTT | | | agar- |
| | | | GAATCTC | GAAATT | | | ose, |
| | | | ATACAA | TAACAG | | | 85V, |
| | | | GAAGAT | ACCAGA | | | 25C |
| | | | AGATC- | TCC-3′ | | | |
| | | | 3′ | [58] | | | |
| | | | [57] | | | | |
| CAPS | CS23 | fad2- | 5′- | 5′- | BamHI | 55 | 1% |
| | 62 | 1 | AACCAC | CCTCAT | | | agar- |
| | | | CGTTCAC | AACTTA | | | ose, |
| | | | GCTGGG | TTGTTG | | | 100V, |
| | | | ATATC- | TACC- | | | 25C |
| | | | 3′ | 3′ | | | |
| | | | [59] | [60] | | | |
| dCAP | CS28 | fad3- | 5′- | 5′- | PstI | 73- | 1% |
| S | 64 | a | CCTTTAC | CATAGT | | 1/ | agar- |
| | | | TGGGCC | GGAACG | | cy- | ose, |
| | | | ATCTTCG | TTACTG | | cle | 100V, |
| | | | TCTGC- | AAAAGT | | (8 | 25C |
| | | | 3′ | G-3′ | | cy- | |
| | | | [61] | [62] | | cles); | |
| | | | | | | 65 | |
| | | | | | | (25 | |
| | | | | | | cy- | |
| | | | | | | cles) | |
| CAPS | CS28 | fae1- | 5′- | 5′- | GsuI/ | 56 | 3% |
| | 64 | a | TTATTGC | CGATCT | BpmI | | agar- |
| | | | AGTTACC | CCTGGC | | | ose, |
| | | | CCCTTAT | TTGTTG | | | 60V, |
| | | | AGGTTTG | GAGAGC | | | 4C |
| | | | G-3′ | AAAA- | | | |
| | | | [63] | 3′ | | | |
| | | | | [64] | | | |
| dCAP | CS29 | fae1- | 5′- | 5′- | BamHI | 59 | 3% |
| S | 01 | c | GATGGT | GGTATG | | | agar- |
| | | | CGTTAAC | AGTCCG | | | ose, |
| | | | ACTTTCA | AACCGT | | | 60V, |
| | | | GGATC- | GTG-3′ | | | 4C |
| | | | 3′ | [66] | | | |
| | | | [65] | | | | |

### Oxidative Stability Index Calculations

Oxidative stability index (OSI) was calculated based on bis-allylic position equivalences (BAPE) that have been shown to correlate well with experimental values (Knothe and Dunn, 2003). BAPE for different fatty acid compositions were calculated based on the 18:2 and 18:3 content.

### Example 1—Isolation of Camelina Mutants with Altered Fatty Acid Composition

M₂ seeds from a Camelina sativa (cultivar Ames 1043) mutant population created by ethyl methanesulfonate (EMS) treatment were analyzed for altered fatty acid composition using gas chromatography (GC). Among the 1,000 lines analyzed, four different phenotypic classes of interest were identified: 1) more 18:1 and less 20:1 than wild type, suggesting possible mutations in FAE1, 2) more 18:1 and less 18:2 than wild type, suggesting possible mutations in FAD2, 3) more 18:2 and less 18:3 than wild type, suggesting possible mutations in FAD3, and 4) more 16:0 and less 18:0 than wild type, suggesting possible mutations in FAB1 encoding KASII. One TILLING line from each of the four mutant classes was chosen for further analysis.

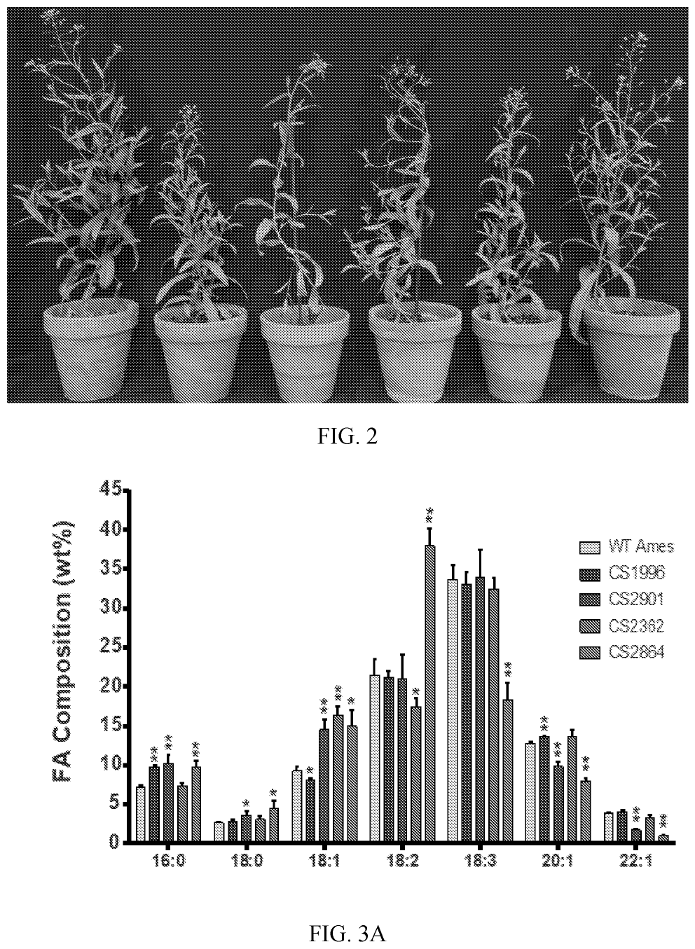

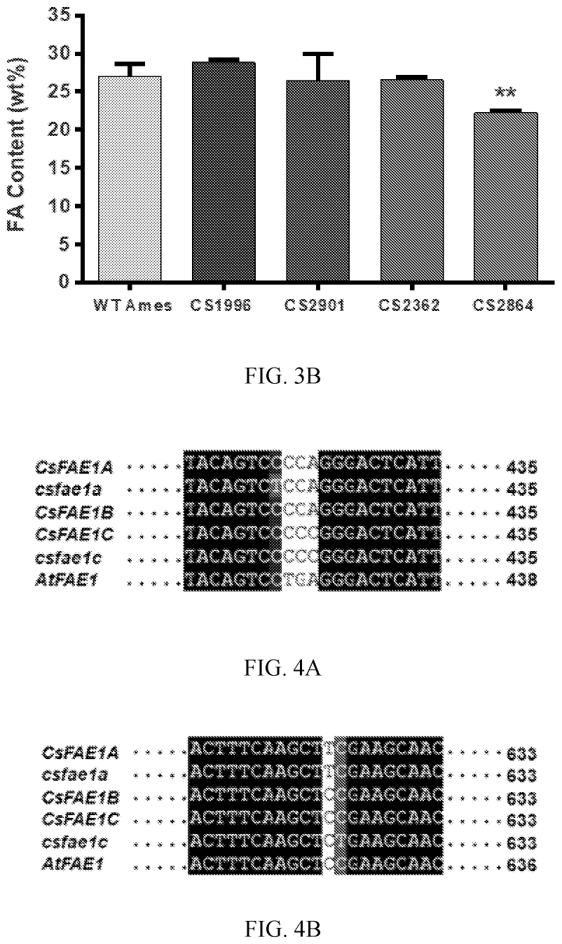

Specific lines were selected based on the magnitude of their fatty acid compositional phenotype, as well as whether they most closely resembled the growth of wild type plants when grown together in the greenhouse (FIG. 2). M₂ seeds from the four chosen lines were planted and allowed to self-pollinate. The fatty acid content and composition of the resulting M₃ seeds were analyzed to confirm the heritability of the phenotype observed in the M₂ seed (FIG. 3A). Seeds from line CS2901 had 10% C20:1, lower than the 13% found in wild type, but 18% 18:1, double the levels present in wild type. Seeds from line CS2362 had decreased amounts of 18:2 (16%) compared to wild type (21%), with an increase of 18:1 from 9% to 15% in the mutant. Seeds from line CS2864 had decreased amounts of 20:1 (8%) compared to wild type (13%) and increased amounts of 18:1 (17%) compared to wild type (9%). This mutant also had a significant decrease of 18:3 (21%) compared to wild type (34%) and an increase in the amount of 18:2. Seeds from line CS1996 contained 9.8% 16:0 compared to 7.5% in wild type. Differences in total fatty acid content were observed in seeds of some of the lines, particularly for CS2864 which was ˜80% that of wild type (FIG. 3B).

### Example 2—Identification of FAE1, FAD2, FAD3 and FAB1 Mutations

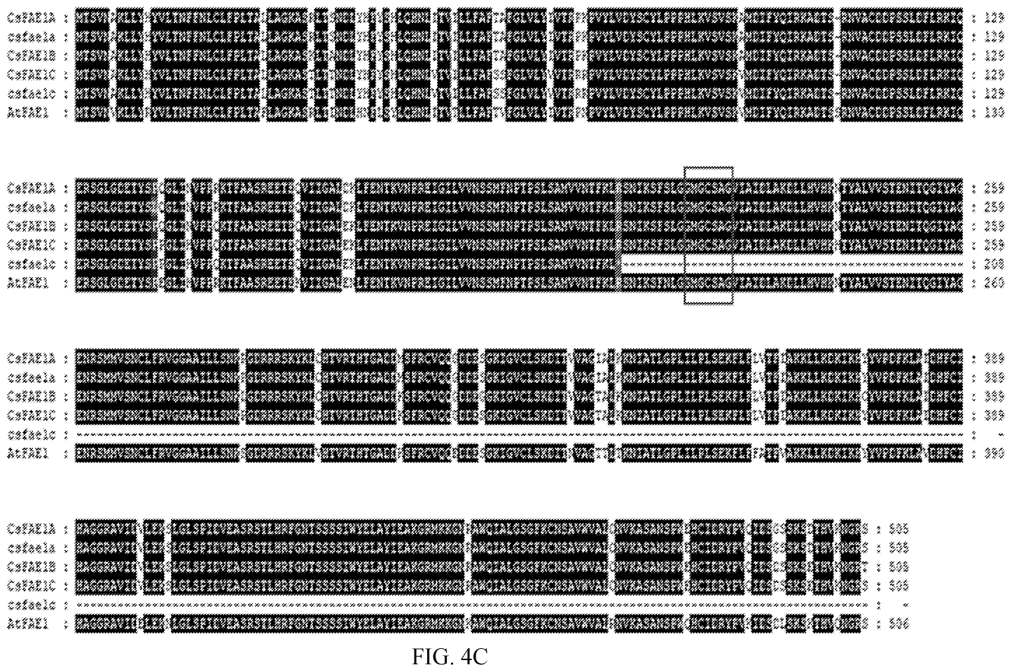

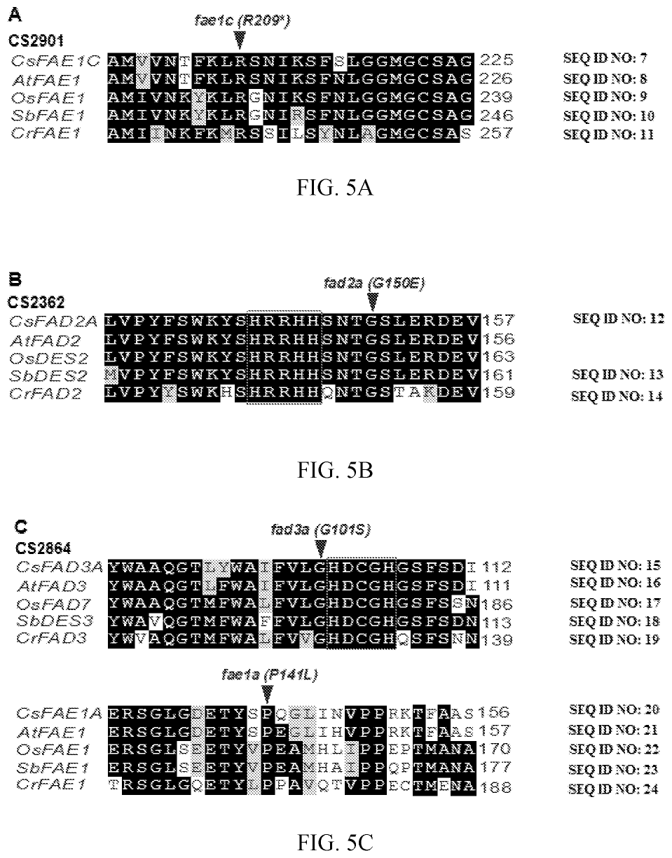

Based on its reduced levels of 20:1, CS2901 may contain a mutation in one of the FAE1 homeologs encoding part of the enzyme complex that catalyzes VLCFA synthesis by elongation of 18:1 to 20:1. Arabidopsis fae1 mutants synthesize very little VLCFA and have increased amounts of 18:1 (James Jr, 1990; Lemieux et al., 1990). Further, the simultaneous mutagenesis of all three camelina FAE1 homeologs using CRISPR-based genome editing results in 20:1 levels of less than 1% with concomitant increases in 18:1 (Ozseyhan et al., 2018). Cloning and sequencing of the three camelina FAE1 homeologs revealed that CS2901 had wild type alleles of FAE1A (Csa11g007400) and FAE1B (Csa10g007610), but FAE1C (Csa12g009060) contains a C625T mutation (FIG. 4A) which replaces an arginine residue with a stop codon (FIGS. 5A and 4C). This nonsense mutation results in a truncated protein lacking the region highly conserved among condensing enzymes in VLCFA biosynthesis.

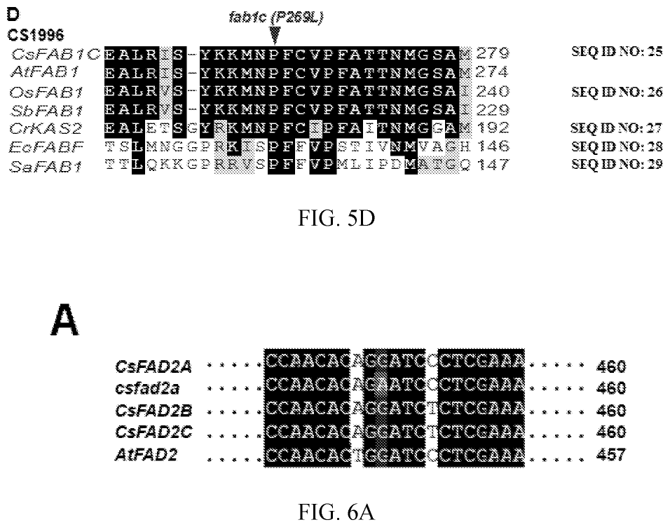

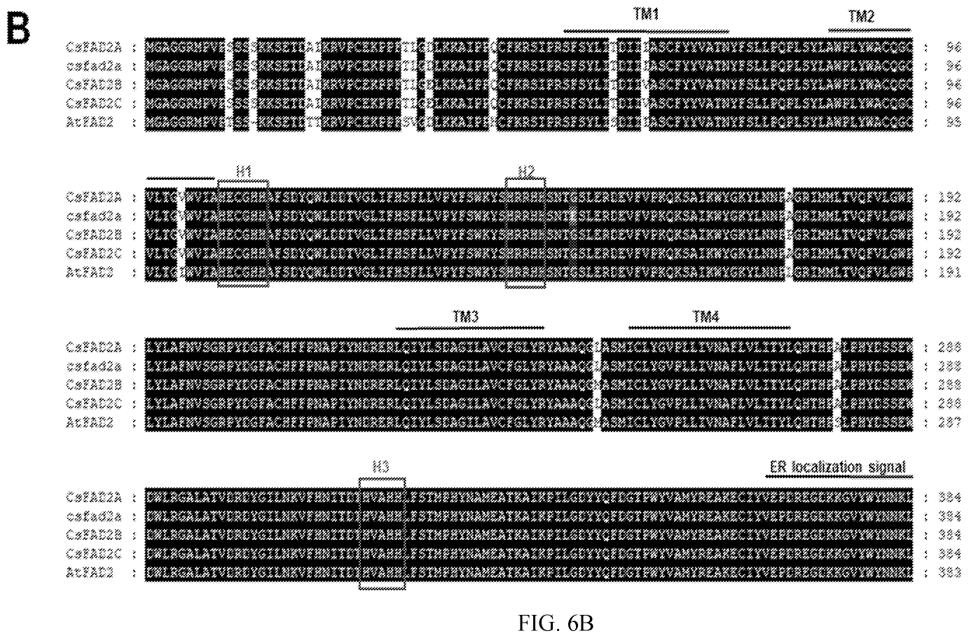

Due to its higher 18:1 levels, CS2362 may have a mutation similar to that affecting the activity of FAD2, the A12-desaturase that catalyzes the synthesis of 18:2 from 18:1 in Arabidopsis (Okuley et al., 1994). Similar to CS2362, Arabidopsis fad2 mutants possess increased levels of 18:1 and lower levels of 18:2 and 18:3 (Lemieux et al., 1990; Okuley et al., 1994). Likewise, genome edited camelina lines with targeted mutations in multiple FAD2 homeologs show higher levels of 18:1 and reduced PUFA content (Jiang et al., 2017; Morineau et al., 2017). Sequencing of all three camelina FAD2 homeologs demonstrated that CS2362 possessed wild type alleles of FAD2B (Csa15g016000) and FAD2C (Csa19g016350), but FAD2A (Csa01g013220) contained a G449A nucleotide change (FIG. 6A), which results in the changed of a conserved glycine residue to glutamate (FIG. 5B). This G150E mutation lies close to the second of three conserved histidine boxes that are critical to the function of the enzyme (FIGS. 5B and 6B) and is predicted by PROVEAN (Protein Variation Effect Analyzer) to be deleterious (Choi et al., 2012).

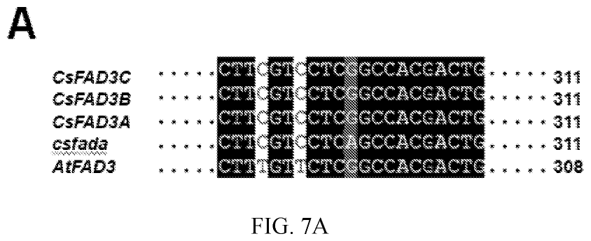

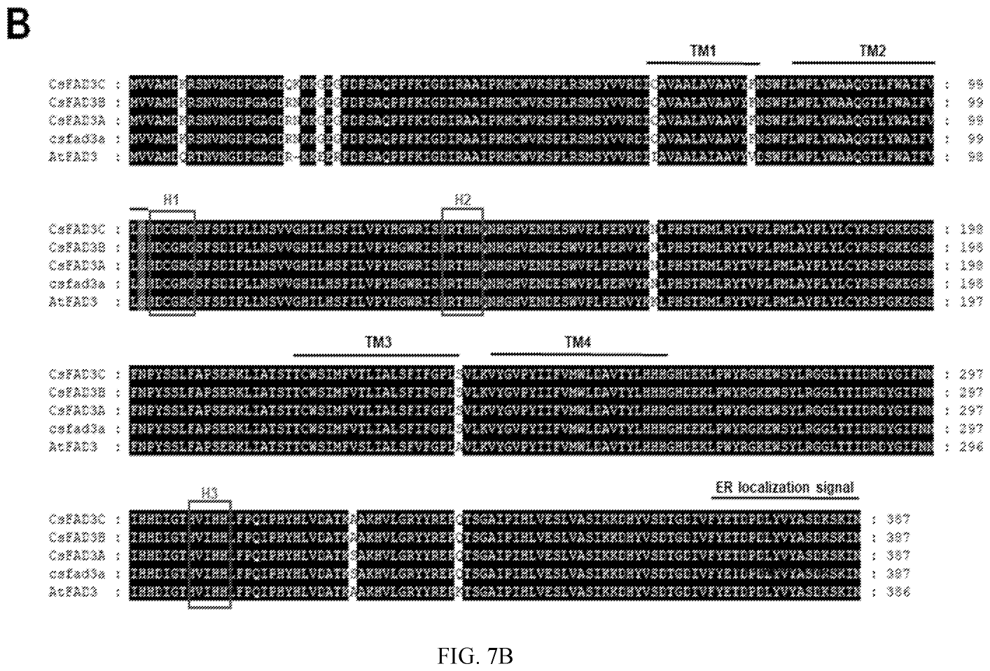

The reduced levels of 18:3 in Arabidopsis fad3 mutants are caused by mutations in the A15-desaturase (Arondel et al., 1992; Yadav et al., 1993). Line CS2864 was therefore suspected of having a mutation in one the FAD3 homeologs, based on its lower levels of 18:3. CS2864 possessed wild type alleles of FAD3C (Csa05g033930) and FAD3B (Csa07g013360), but FAD3A (Csa16g014970) contained a G301A mutation (FIG. 7A), resulting in a G101S substitution. This mutation affects a conserved residue present in the second transmembrane domain of FAD3 and is located adjacent to one of the histidine boxes important for enzyme function (FIGS. 5C and 7B). The decreased levels of C20:1 in seeds of CS2864 suggested that this line might also have a mutation in one of the FAE1 homeologs. Sequencing demonstrated that FAE1A in CS2864 contained a C422T nucleotide change (FIG. 4B) with the consequent replacement of a conserved proline residue with leucine (FIGS. 5C and 4C). FAE1B and FAE1C did not possess any mutations in this line. The affected proline is conserved and leucine substitution is predicted by PROVEAN to be deleterious.

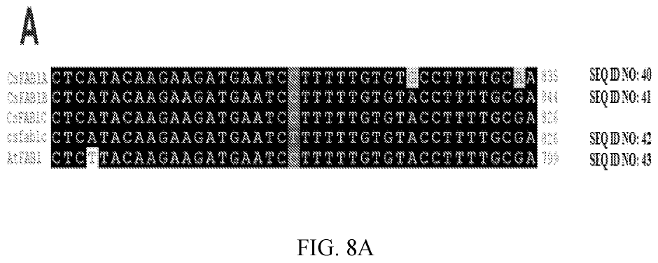

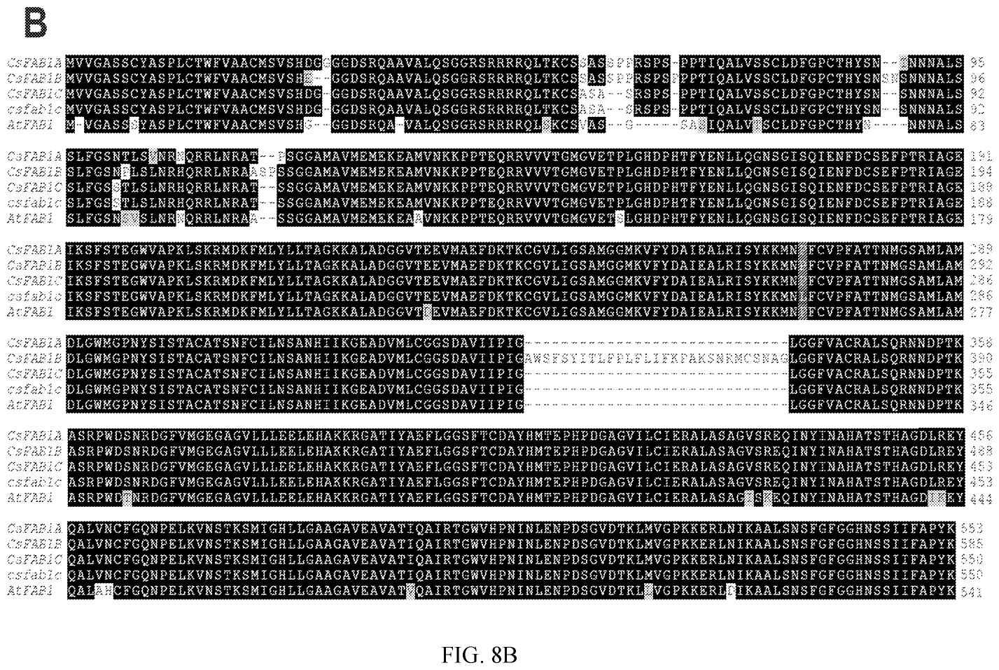

Sequencing demonstrated a C1425T mutation in the fifth exon of FAB1C (Csa09g079550) resulting in a P269L substitution (FIG. 8). The other FAB1 homologs Csa16g038860 (FAB1A) and Csa07g046400 (FAB1B) contained no mutations. The mutated proline residue of fab1c is highly conserved in orthologs from diverse species, including fabF from E. coli (FIG. 5D), suggesting an essential function for this particular residue. Further, the P269L substitution is predicted by PROVEAN to be deleterious. GenBank accession numbers for the proteins used in the alignments are located in Table 3.

TABLE 3
Gene Bank IDs

| | | | GenBankID | |
|---|---|---|---|---|
| Organisms | FAE1 | FAD2 | FAD3 | FA Bl |
| Camelina sativa | ADN10812, ADN10814 | ADN10824 | XP_010469908 | NP_001306958 |
| Arabidopsis thaliana | NP_195178 | NP_001319529 | NP_180559 | NP_001185400 |
| Oryza sativa | XP_015639811 | XP_015625626 | XP_015627771 | XP_015644677 |
| Sorghum bicolor | XP_002441534 | XP_002452649 | XP_002450090 | XP_002463181 |
| Chlamydomonas reinhardtii | PNW70493 | XP_001691669 | ABL09485 | XP_001700152 |
| Escherichia coli | X | X | X | WP_072662266 |
| Staphylococcus aureus | X | X | X | WP_075109203 |

The partial changes in fatty acid composition in these camelina mutants compared to orthologous mutations in Arabidopsis reflect the fact that the three camelina subgenomes are highly related and undifferentiated (Kagale et al., 2014). Previous work has demonstrated that all three FAE1 and FAD2 homeologs are expressed in developing seeds (Hutcheon et al., 2010). Consistent with this, camelina lines with mutations in individual FAE1 or FAD2 homeologs only possessed a partial phenotype whereas lines with mutations in multiple homeologs presented a stronger phenotype.

### Example 3—Functional Expression of Wild Type and Mutant Alleles in Yeast Cells

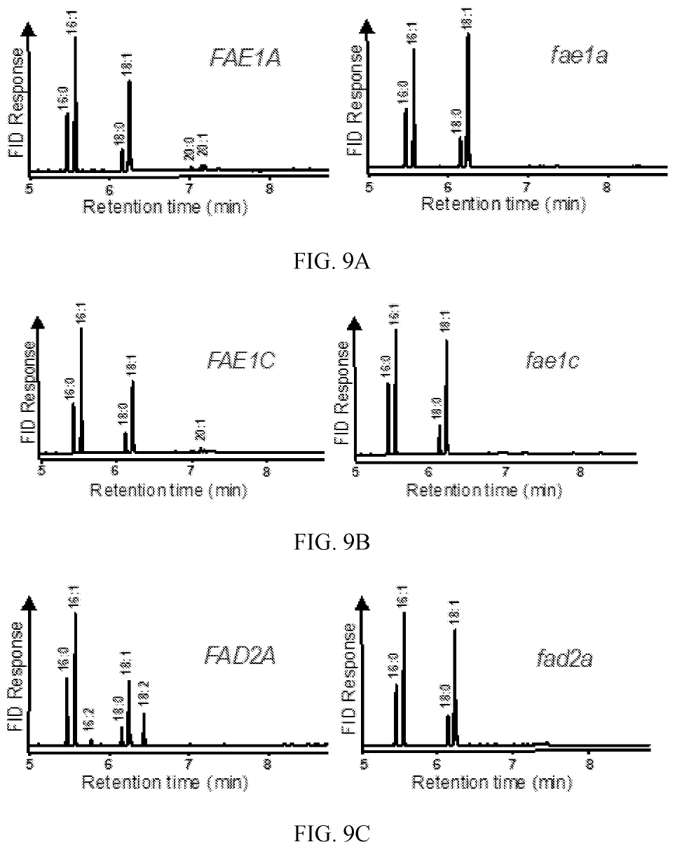

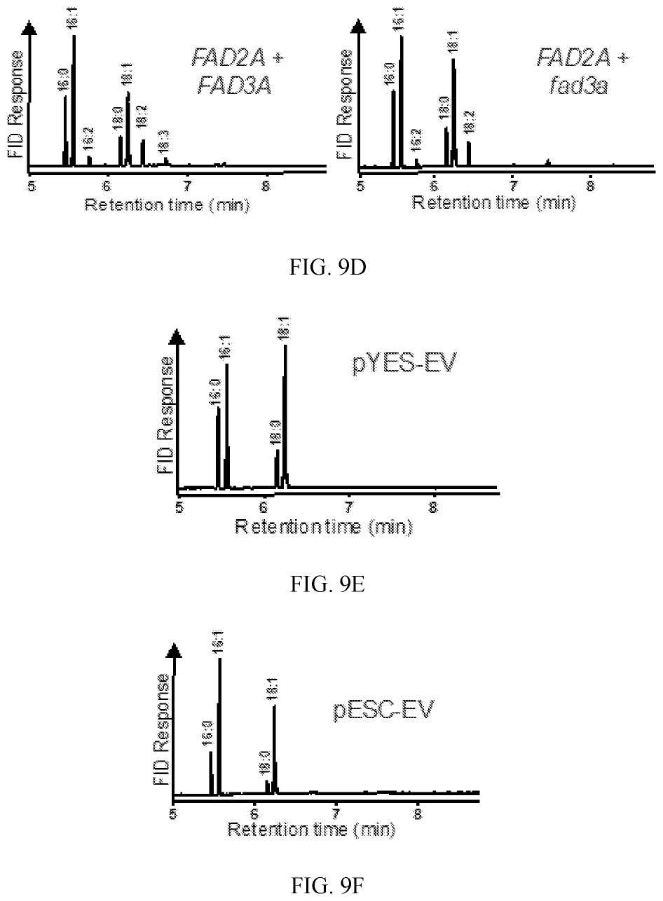

The wild type and mutated alleles of FAE1A, FAE1C, FAD2A and FAD3A were expressed in yeast to determine if the mutations had an effect on the activity of the enzymes and therefore were responsible for the altered fatty acid composition observed in the mutant lines. In the control yeast cells transformed with the empty pYES2 vector, no VLCFAs were produced (FIG. 9E). However, yeast cells expressing wild type FAE1A were able to elongate 18:0 into arachidic (20:0) and behenic (22:0) acids, as well as 18:1 into 20:1 and C22:1, consistent with the activity of FAE1 (FIG. 9A). However, when the fae1a mutant allele from CS2864 was expressed, we did not detect any VLCFA (FIG. 9A). Similarly, the expression of the wild type FAE1C allele resulted in 20:1 synthesis, but not with the expression of the fae1c mutant allele from CS2901 (FIG. 9B). These results suggest these two camelina mutant fae1 alleles encode non-functional enzymes.

Normally no polyunsaturated acids are produced by S. cerevisiae (FIGS. 9E and 9F). However, cells expressing wild type FAD2A synthesized hexadecadienoic acid (16:2) and 18:2 from the action of the Δ12-desaturase on palmitoleic acid (16:1) and 18:1, respectively. In contrast, no 16:2 or 18:2 was detected with the expression of the fad2a mutant allele from CS2362 (FIG. 9C) suggesting the encoded desaturase lacks activity.

In the yeast cells expressing wild type copies of both FAD2A and FAD3A, there is production of 16:2 and 18:2 from the activity of the FAD2A A12 desaturase and then conversion of 18:2 to 18:3 due to the activity of FAD3A. However, when the mutant fad3a allele from CS2864 is expressed in combination with FAD2A, there is a major reduction of 18:3, suggesting this mutation substantially affects the activity of the encoded enzyme (FIG. 9D).

### Example 4—the FAB1 Mutant Allele Segregates with Increased 16:0 Content

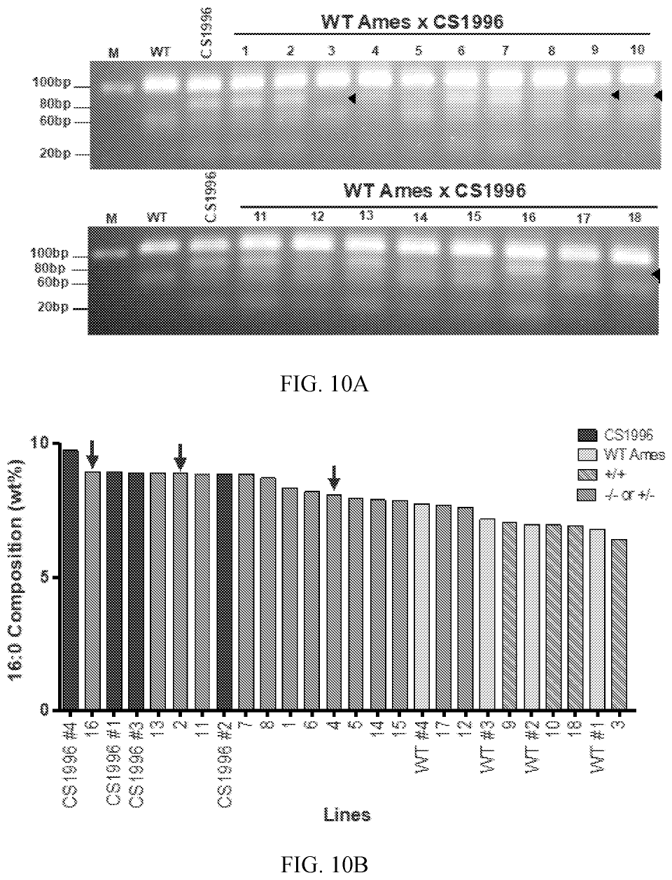

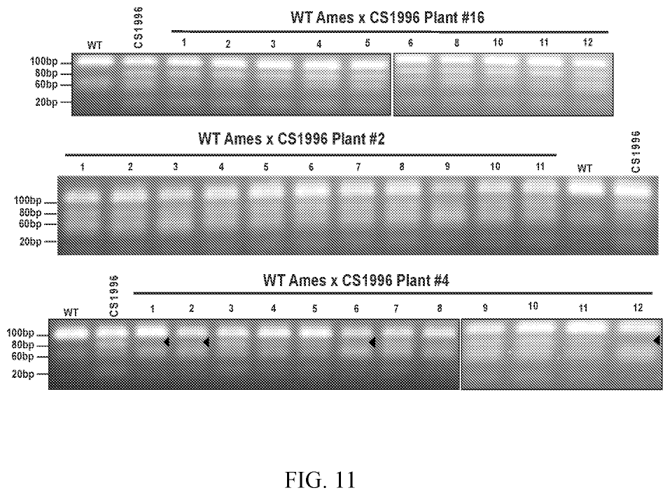

KASII functions in a Type II fatty acid synthase in plant cells, complicating its expression in yeast, which uses a Type I fatty acid synthase. To confirm that the fab1c allele in CS1996 is responsible for the high 16:0 phenotype of the mutant seed, the mutant line was backcrossed to the Ames 1043 wild type background. When 18 F₂ plants were genotyped, 14 possessed the C1425T mutation and 4 were wild type (FIG. 10A). As the fab1c dCAPs marker (Table 2) is unable to distinguish the three highly identical camelina FAB1 homeologs, we could not differentiate plants that were homozygous for the mutation from those that were heterozygous. Quantification of 16:0 levels in the F₃ seed harvested from these plants revealed that the F₂ plants with a wild type genotype possessed lower levels of 16:0, comparable with those of control wild type seeds (FIG. 10B). Seed from six of the F₂ plants containing the C1425T fab1c mutation had levels of 16:0 similar to that of CS1996 plants. Eight F₂ plants produced seed containing levels of 16:0 intermediate between the CS1996 and wild type controls. Thus, the high 16:0 plants may be homozygous for the fab1c mutation, with the intermediate 16:0 plants being heterozygous. F₃ progeny of selected plants were genotyped to discover whether they were homozygous or heterozygous for the mutation. From two plants producing high levels of 16:0, all derived F₃ plants contained the C1425T mutation (FIG. 11).

The result demonstrates that the F₂ parents are homozygous for the mutation, consistent with the high 16:0 phenotype. F₃ plants derived from the seed containing intermediate amounts of 16:0 segregated 3:1 for progeny possessing the mutation versus those that only possess wild type homeologs of FAB1. Therefore, these F₂ plants were heterozygous for the mutation, explaining the intermediate C16:0 levels.

### Example 5—Complementation of Altered Fatty Acid Composition in Camelina TILLING Lines

To determine if the altered fatty acid composition in mutant lines could be complemented or restored to wild-type amounts, cDNAs were cloned for wild type alleles of FAE1A, FAE1C, FAD2A or FAD3A under the control of the seed-specific glycinin-1 promoter. The fluorescent protein DsRed was used as the selectable marker. Each mutant line was transformed with the pertinent binary construct by vacuum infiltration of Agrobacterium tumefaciens. Five T₁ red seeds were then selected for fatty acid composition analysis by GC, as well as seeds from the mutant line transformed with the empty binary vector and from wild type plants grown at the same time.

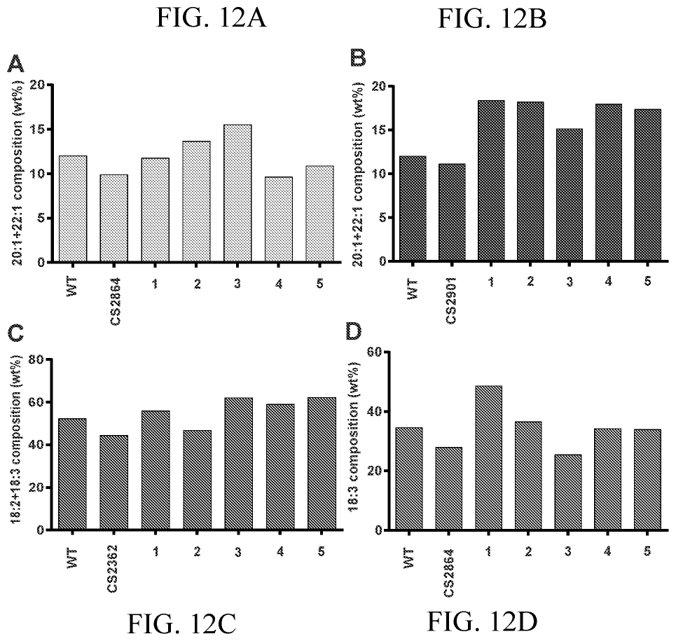

The mutant line CS2864, containing the fae1a allele, had around 10% VLCFA (20:1+22:1), lower than wild type levels of 12%. In contrast, seeds of four of the five T₁ lines contained levels higher than the mutant and two of them levels above wild type, up to 16% (FIG. 12A). Similarly, expression of a wild type copy of FAE1C in CS2901 increased the levels of VLCFA from 11% up to 18% in the five T₁ lines (FIG. 12B). The PUFA (18:2+18:3) levels of CS2362, which contains a mutation in FAD2A, are 44% compared to wild type levels of 52%. All five T₁ lines expressing FAD2A contained levels higher than the mutant, with four of them having levels above wild type, up to 62% (FIG. 12C). Likewise, the reduced 18:3 content of seeds from CS2864, containing the fad3a mutant, was complemented in four of the five T₁ lines expressing FAD3A, with one line containing 18:3 levels up to 49% (FIG. 12D). These results further confirm that the mutant alleles are responsible for the changes in seed fatty acid composition seen in the camelina mutants.

### Example 6—Combining Mutations to Create Mid-Oleic Camelina Lines

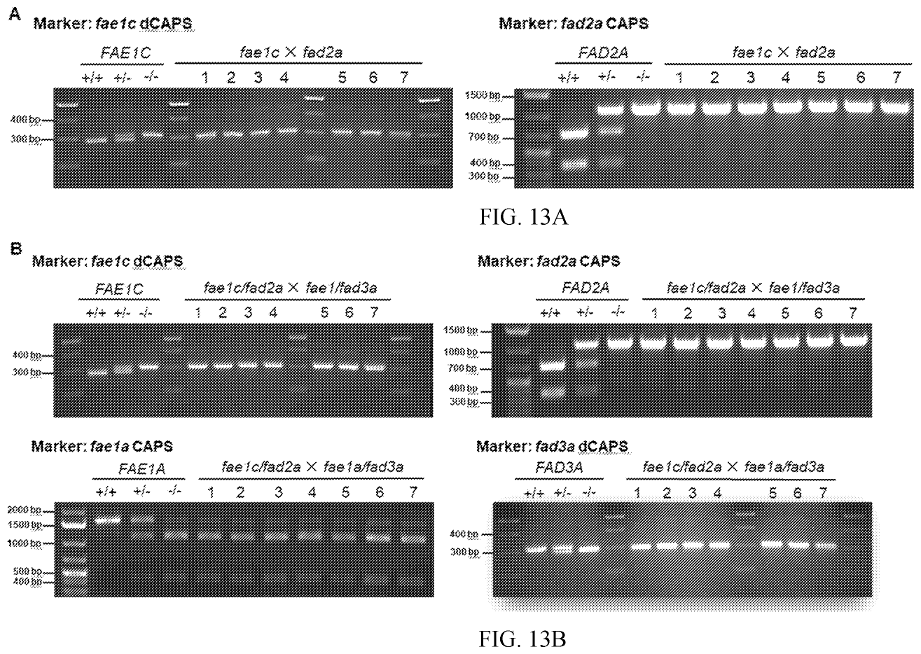

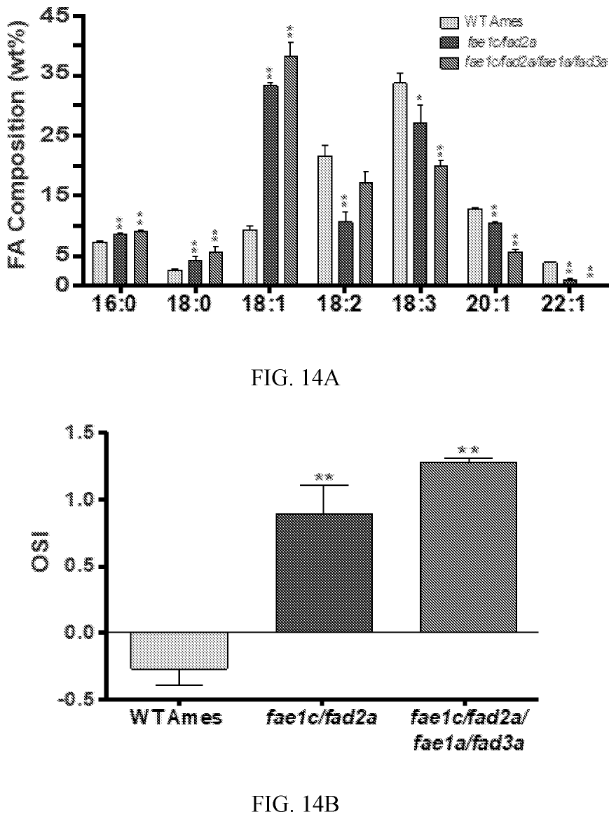

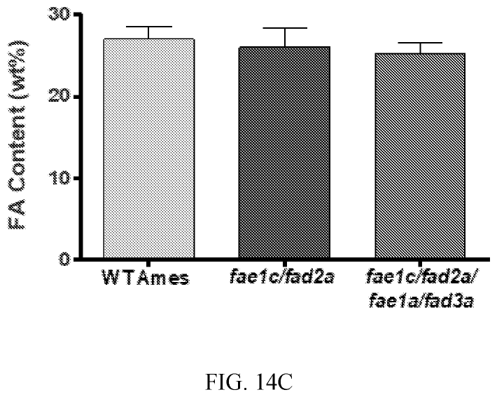

An oil with increased oleic and decreased polyunsaturated fatty acid (PUFA) content is more desirable as a biodiesel feedstock because of the improved oxidative stability and ignition quality conferred by these changes in fatty acid composition. Likewise, a decrease in VLCFAs allows better cold flow temperature properties. An improved fatty acid composition such as this can be achieved, according to the subject invention, by downregulating FAE1, FAD2 and FAD3 activity. Thus, the fae1c mutant, CS2901, was crossed with the fad2a mutant, CS2362. F₃ plants homozygous for both mutations were identified by genotyping (FIG. 13) and the fatty acid phenotype confirmed in the seed (FIG. 14A). The fae1c/fad2a mutant cross has decreased levels of 20:1,18:2 and 18:3 due to the two combined mutations, thereby increasing the amount of oleic acid from 9% in wild type to 34% in the new line (FIG. 14A). The reduced PUFA content in the fae1c/fad2a line results in an increased oxidative stability index (OSI), as calculated according to the bis-allylic position equivalences (BAPE) of the fatty acid composition (Knothe and Dunn, 2003). Here, the OSI for oil from fae1c/fad2a resulted in an OSI of 0.90, much greater than that for wild type camelina oil, which is slightly negative (FIG. 14B).

To further alter seed fatty acid composition, the fae1c/fad2a line was crossed with CS2864, which contains mutations in FAE1A and FAD3A. Plants homozygous for all four mutations were identified in the F₅ generation by genotyping (FIG. 13B). The amount of 20:1 was reduced even further with the combination of the fae1a allele with fae1c (FIG. 14A). Levels of 18:3 were reduced even further with the addition of the fad3a mutant alleles, raising the amount of oleic acid to 43%.

Consequently, the calculated OSI for the quadruple mutant combination increased to 1.28 (FIG. 14B). The fae1c/fad2a line was slightly smaller than wild-type plants when grown in the greenhouse, but the quadruple mutant appeared phenotypically normal (FIG. 2). Both lines containing mutant combinations demonstrated slight reductions in total seed fatty acid content (FIG. 14C).

All publications, patents, and patent publications cited are incorporated by reference herein in their entirety for all purposes. Also incorporated by reference herein are nucleic acid sequences and polypeptide sequences deposited into the GenBank, which are cited in this specification.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

We claim:

1. A polynucleotide encoding a plant fatty acid synthesis enzyme, wherein the polynucleotide has one or more mutations such that the polynucleotide encodes a plant fatty acid synthesis enzyme that is mutated compared to a wild-type fatty acid synthase, wherein the expression of said mutated enzyme in a plant results in a reduction in polyunsaturated fatty acids and/or long chain fatty acids in the plant expressing the mutant enzyme compared to a plant expressing the wild-type enzyme.

2. The polynucleotide of claim 1, wherein the plant fatty acid synthesis enzyme is a plant fatty acid desaturase, a plant fatty acid elongase, or a plant beta-ketoacyl-ACP synthase.

3. The polynucleotide of claim 2, wherein the plant fatty acid desaturase is FAD2 or FAD3.

4. The polynucleotide of claim 2, wherein the plant fatty acid elongase is FAE1.

5. The polynucleotide of claim 2, wherein the beta-ketoacyl-ACP synthase is KASII.

6. The polynucleotide of claim 1, wherein said one or more mutations occur in a nucleic acid sequence selected from the group consisting of SEQ ID NOs: 67 to 78.

7. The polynucleotide of claim 3, wherein the polynucleotide sequence encoding the plant fatty acid desaturase comprises a mutation selected from G301A in the FAD3A gene and G449A in the FAD2A gene.

8. The polynucleotide of claim 4, wherein the polynucleotide sequence encoding the plant fatty acid elongase comprises a mutation selected from C625T in the FAE1C gene and C422T in the FAE1A gene.

9. A plant fatty acid synthesis enzyme, having one or more mutations, compared to a wild-type enzyme, wherein the expression of said mutant enzyme in a plant results in a reduction in polyunsaturated fatty acids and/or long chain fatty acids in the plant expressing the mutant enzyme compared to a plant expressing the wild-type enzyme.

10. The plant fatty acid synthesis enzyme of claim 9, wherein the mutated enzyme comprises an amino acid sequence that is mutated with one or more amino acid mutations compared to an amino acid sequence selected from the group consisting of SEQ ID NOs: 1, 3-4, 30, 32-33, 35-37, and 44-46.

11. The polypeptide of claim 9, wherein the plant fatty acid synthesis enzyme is a plant fatty acid desaturase, a plant fatty acid elongase, or a plant beta-ketoacyl-ACP synthase.

12. The polypeptide of claim 11, wherein the plant fatty acid desaturase is FAD2 or FAD3.

13. The polypeptide of claim 11, wherein the plant fatty acid elongase is FAE1.

14. The polypeptide of claim 11, wherein the beta-ketoacyl-ACP synthase is KASII.

15. The polypeptide of claim 12, wherein the plant fatty acid desaturase comprises one or more mutations selected from G150E in FAD2A and G101S in FAD3A.

16. The polypeptide of claim 13, wherein the plant fatty acid elongase comprises one or more mutations selected from R209* in FAE1 C and P141L in FAE1A.

17. The polypeptide of claim 9, wherein the polypeptide comprises an amino acid sequence selected from the group consisting of SEQ ID NOs: 2, 5, 31, 38, and 47.

18. A plant cell, plant part, tissue culture or whole plant comprising a polynucleotide according to claim 1.

19. The plant cell, plant part, tissue culture or whole plant of claim 18, wherein the plant cell, plant part, tissue culture or whole plant comprises at least two FAE1 genes, at least one FAD3 gene, and at least one FAD2 gene, each of which genes encodes a fatty acid synthesis enzyme that is mutated compared to a wild-type fatty acid synthesis gene.

20. The plant cell, plant part, tissue culture or whole plant of claim 18, wherein the plant cell, plant part, tissue culture or whole plant comprises FAE1A, FAE1C, FAD2A, and FAD3A genes, each of which comprises at least one mutation such that the gene encodes a fatty acid synthesis enzyme that is mutated compared to a wild-type fatty acid synthesis gene that disrupts and/or alters the function of the gene.

21. The plant cell, plant part, tissue culture or whole plant of claim 18, further comprising a mutated fatty acid gene that encodes a mutated enzyme selected from the group consisting of FAB1, a hydroxylase and a thioesterase.

22. The plant cell, plant part, tissue culture or whole plant of claim 18, wherein said plant cell, plant part, tissue culture, or whole plant expresses a plant fatty acid desaturase having one or more of G150E and G101S mutations, and/or a fatty acid elongase having one or more of R209* and P141L mutations.

23. A method of altering a fatty acid composition of a Camelina plant comprising transforming the plant with a polynucleotide of claim 1.

24. The method of claim 23, wherein one, two or three FAD2 genes, one, two, or three FAD3 genes, and/or one, two or three FAE1 genes are mutated compared to a wild-type gene such that the plant expresses a plant fatty acid synthesis enzyme, having one or more mutations, compared to a wild-type enzyme, wherein the expression of said mutant enzyme in said plant results in a reduction in polyunsaturated fatty acids and/or long chain fatty acids in the plant expressing the mutant enzyme compared to a plant expressing the wild-type enzyme.

25. The method of claim 24, wherein one FAD2 gene, one FAD3 gene and/or two FAE1 genes are mutated compared to a wild-type gene.

26. The method of claim 23, wherein said plant expresses a plant fatty acid desaturase having one or more of G150E and G101S mutations, and/or a fatty acid elongase having one or more of R209* and P141L mutations.

27. A method of increasing the activity of a FAD2, FAD3 and/or FAE1 protein in a Camelina plant cell, plant part, tissue culture or whole plant comprising transforming the plant cell, plant part, tissue culture or whole plant with a polynucleotide of claim 1.


SEQUENCE LISTING (98 sequences)

SEQ ID NO: 1 - protein, 505 aa, Camelina sativa
MTSVNAKLLYHYVLTNFFNLCLFPLTALLAGKASRLTSNDLYHFYSHLQHNLITVILLFAFTAFGLVLYIVTRPKPVYLVDYSCYLPPPHLKVSVSKAMDIFYQIRKADTSRNVACDDPSSLDFLRKIQERSGLGDETYSPQGLINVPPRKTFAASREETEQVIIGALDKLFENTKVNPREIGILVVNSSMFNPTPSLSAMVVNTFKLRSNIKSFSLGGMGCSAGVIAIDLAKDLLHVHKNTYALVVSTENITQGIYAGENRSMMVSNCLFRVGGAAILLSNKPGDRRRSKYKLCHTVRTHTGADDMSFRCVQQGDDESGKIGVCLSKDITVVAGIALKKNIATLGPLILPLSEKFLFLVTFIAKKLLKDKIKHYYVPDFKLAIDHFCIHAGGRAVIDVLEKSLGLSPIDVEASRSTLHRFGNTSSSSIWYELAYIEAKGRMKKGNRAWQIALGSGFKCNSAVWVALCNVKASANSPWEHCIDRYPVQIDSGSSKSDTHVKNGRS

SEQ ID NO: 2 - protein, 505 aa, Artificial sequence
  Camelina sativa mutant
MTSVNAKLLYHYVLTNFFNLCLFPLTALLAGKASRLTSNDLYHFYSHLQHNLITVILLFAFTAFGLVLYIVTRPKPVYLVDYSCYLPPPHLKVSVSKAMDIFYQIRKADTSRNVACDDPSSLDFLRKIQERSGLGDETYSLQGLINVPPRKTFAASREETEQVIIGALDKLFENTKVNPREIGILVVNSSMFNPTPSLSAMVVNTFKLRSNIKSFSLGGMGCSAGVIAIDLAKDLLHVHKNTYALVVSTENITQGIYAGENRSMMVSNCLFRVGGAAILLSNKPGDRRRSKYKLCHTVRTHTGADDMSFRCVQQGDDESGKIGVCLSKDITVVAGIALKKNIATLGPLILPLSEKFLFLVTFIAKKLLKDKIKHYYVPDFKLAIDHFCIHAGGRAVIDVLEKSLGLSPIDVEASRSTLHRFGNTSSSSIWYELAYIEAKGRMKKGNRAWQIALGSGFKCNSAVWVALCNVKASANSPWEHCIDRYPVQIDSGSSKSDTHVKNGRS

SEQ ID NO: 3 - protein, 505 aa, Camelina sativa
MTSVNAKLLYHYVLTNFFNLCLFPLTALLAGKASKLTANDLYHFYSHLQHNLITVILLFAFTAFGLVLYIVTRPKPVYLVDYSCYLPPPHLKVSVSKAMDIFYQIRKADTSRNVACDDPSSLDFLRKIQERSGLGDETYSPQGLINVPPQKTFAASREETEQVIIGALEKLFENTKVNPREIGILVVNSSMFNPTPSLSAMVVNTFKLRSNIKSFSLGGMGCSAGVIAIDLAKDLLHVHKNTYALVVSTENITQGIYAGENRSMMVSNCLFRVGGAAILLSNKLGDRRRSKYKLCHTVRTHTGADDKSFRCVQQGDDEGGKIGVCLSKDITVVAGTALKKNIATLGPLILPLSEKFLFLVTFIAKKLLKDKIKHCYVPDFKLAIDHFCIHAGGRAVIDVLEKSLGLSPIDVEASRSTLHRFGNTSSSSIWYELAYIEAKGRMKKGNRAWQIALGSGFKCNSAVWVALCNVKASANSPWEHCIDRYPVQIDSDSSKSETHVKNGRS

SEQ ID NO: 4 - protein, 505 aa, Camelina sativa
MTSVNAKLLYHYVLTNFFNLCLFPLTALLAGKASTLTTNDLYHFYSHLQHNLVTVILLFAFSSFGLVLYVVTRRRPVYLVDYSCYLPPPHLKVSVSKVMDIFYQIRKADTSRNVACDDPSSLDFLRKIQERSGLGDETYSPPGLIHVPPQKTFAASREETEQVIIGALEKLFENTKVNPREIGILVVNSSMFNPTPSLSAMVVNTFKLRSNIKSFSLGGMGCSAGVIAIDLAKDLLHVHKHTYALVVSTENITQGIYAGENRSMMVSNCLFRVGGAAILLSNKPGDRRRSKYKLCHTVRTHTGADDKSFRCVQQGDDESGKIGVCLSKDITVVAGTALKKNIATLGPLILPLSEKFLFLVTFIAKKLLKDKIKHYYVPDFKLAIDHFCIHAGGRAVIDVLEKSLGLSPIDVEASRSTLHRFGNTSSSSIWYELAYIEAKGRMKKGNRAWQIALGSGFKCNSAVWVALCNVKASANSPWEHCIDRYPVQIDSDSSKSETHVKNGRS

SEQ ID NO: 5 - protein, 208 aa, Artificial sequence
  Camelina sativa mutant
MTSVNAKLLYHYVLTNFFNLCLFPLTALLAGKASTLTTNDLYHFYSHLQHNLVTVILLFAFSSFGLVLYVVTRRRPVYLVDYSCYLPPPHLKVSVSKVMDIFYQIRKADTSRNVACDDPSSLDFLRKIQERSGLGDETYSPPGLIHVPPQKTFAASREETEQVIIGALEKLFENTKVNPREIGILVVNSSMFNPTPSLSAMVVNTFKL

SEQ ID NO: 6 - protein, 506 aa, Arabidopsis thaliana
MTSVNVKLLYRYVLTNFFNLCLFPLTAFLAGKASRLTINDLHNFLSYLQHNLITVTLLFAFTVFGLVLYIVTRPNPVYLVDYSCYLPPPHLKVSVSKVMDIFYQIRKADTSSRNVACDDPSSLDFLRKIQERSGLGDETYSPEGLIHVPPRKTFAASREETEKVIIGALENLFENTKVNPREIGILVVNSSMFNPTPSLSAMVVNTFKLRSNIKSFSLGGMGCSAGVIAIDLAKDLLHVHKNTYALVVSTENITQGIYAGENRSMMVSNCLFRVGGAAILLSNKSGDRRRSKYKLVHTVRTHTGADDKSFRCVQQEDDESGKIGVCLSKDITNVAGTTLTKNIATLGPLILPLSEKFLFFATFVAKKLLKDKIKHYYVPDFKLAVDHFCIHAGGRAVIDELEKNLGLSPIDVEASRSTLHRFGNTSSSSIWYELAYIEAKGRMKKGNKAWQIALGSGFKCNSAVWVALRNVKASANSPWQHCIDRYPVKIDSDLSKSKTHVQNGRS

SEQ ID NO: 7 - protein, 26 aa, Camelina sativa
AMVVNTFKLRSNIKSFSLGGMGCSAG

SEQ ID NO: 8 - protein, 26 aa, Arabidopsis thaliana
AMVVNTFKLRSNIKSFNLGGMGCSAG

SEQ ID NO: 9 - protein, 26 aa, Oryza sativa
AMIVNKYKLRGNIKSFNLGGMGCSAG

SEQ ID NO: 10 - protein, 26 aa, Sorghum bicolor
AMIVNKYKLRGNIRSFNLGGMGCSAG

SEQ ID NO: 11 - protein, 26 aa, Chlamydomonas reinhardtii
AMIINKFKMRSSILSYNLAGMGCSAS

SEQ ID NO: 12 - protein, 26 aa, Camelina sativa
LVPYFSWKYSHRRHHSNTGSLERDEV

SEQ ID NO: 13 - protein, 26 aa, Sorghum bicolor
MVPYFSWKYSHRRHHSNTGSLERDEV

SEQ ID NO: 14 - protein, 26 aa, Chlamydomonas reinhardtii
LVPYYSWKHSHRRHHQNTGSTAKDEV

SEQ ID NO: 15 - protein, 27 aa, Camelina sativa
YWAAQGTLYWAIFVLGHDCGHGSFSDI

SEQ ID NO: 16 - protein, 27 aa, Arabidopsis thaliana
YWAAQGTLFWAIFVLGHDCGHGSFSDI

SEQ ID NO: 17 - protein, 27 aa, Oryza sativa
YWAAQGTMFWALFVLGHDCGHGSFSSN

SEQ ID NO: 18 - protein, 27 aa, Sorghum bicolor
YWAVQGTMFWAFFVLGHDCGHGSFSDN

SEQ ID NO: 19 - protein, 27 aa, Chlamydomonas reinhardtii
YWVAQGTMFWALFVVGHDCGHQSFSNN

SEQ ID NO: 20 - protein, 27 aa, Camelina sativa
ERSGLGDETYSPQGLINVPPRKTFAAS

SEQ ID NO: 21 - protein, 27 aa, Arabidopsis thaliana
ERSGLGDETYSPEGLIHVPPRKTFAAS

SEQ ID NO: 22 - protein, 27 aa, Oryza sativa
ERSGLSEETYVPEAMHLIPPEPTMANA

SEQ ID NO: 23 - protein, 27 aa, Sorghum bicolor
ERSGLSEETYVPEAMHAIPPQPTMANA

SEQ ID NO: 24 - protein, 27 aa, Chlamydomonas reinhardtii
TRSGLGQETYLPPAVQTVPPECTMENA

SEQ ID NO: 25 - protein, 26 aa, Camelina sativa
EALRISYKKMNPFCVPFATTNMGSAM

SEQ ID NO: 26 - protein, 26 aa, Oryza sativa
EALRVSYKKMNPFCVPFATTNMGSAI

SEQ ID NO: 27 - protein, 27 aa, Chlamydomonas reinhardtii
EALETSGYRKMNPFCIPFAITNMGGAM

SEQ ID NO: 28 - protein, 27 aa, Escherichia coli
TSLMNGGPRKISPFFVPSTIVNMVAGH

SEQ ID NO: 29 - protein, 27 aa, Staphylococcus aureus
TTLQKKGPRRVSPFFVPMLIPDMATGQ

SEQ ID NO: 30 - protein, 384 aa, Camelina sativa
MGAGGRMPVPSSSSKKSETDAIKRVPCEKPPFTLGDLKKAIPPQCFKRSIPRSFSYLITDIIIASCFYYVATNYFSLLPQPLSYLAWPLYWACQGCVLTGVWVIAHECGHHAFSDYQWLDDTVGLIFHSFLLVPYFSWKYSHRRHHSNTGSLERDEVFVPKQKSAIKWYGKYLNNPAGRIMMLTVQFVLGWPLYLAFNVSGRPYDGFACHFFPNAPIYNDRERLQIYLSDAGILAVCFGLYRYAAAQGLASMICLYGVPLLIVNAFLVLITYLQHTHPALPHYDSSEWDWLRGALATVDRDYGILNKVFHNITDTHVAHHLFSTMPHYNAMEATKAIKPILGDYYQFDGTPWYVAMYREAKECIYVEPDREGDKKGVYWYNNKL

SEQ ID NO: 31 - protein, 384 aa, Artificial sequence
  Camelina sativa mutant
MGAGGRMPVPSSSSKKSETDAIKRVPCEKPPFTLGDLKKAIPPQCFKRSIPRSFSYLITDIIIASCFYYVATNYFSLLPQPLSYLAWPLYWACQGCVLTGVWVIAHECGHHAFSDYQWLDDTVGLIFHSFLLVPYFSWKYSHRRHHSNTESLERDEVFVPKQKSAIKWYGKYLNNPAGRIMMLTVQFVLGWPLYLAFNVSGRPYDGFACHFFPNAPIYNDRERLQIYLSDAGILAVCFGLYRYAAAQGLASMICLYGVPLLIVNAFLVLITYLQHTHPALPHYDSSEWDWLRGALATVDRDYGILNKVFHNITDTHVAHHLFSTMPHYNAMEATKAIKPILGDYYQFDGTPWYVAMYREAKECIYVEPDREGDKKGVYWYNNKL

SEQ ID NO: 32 - protein, 384 aa, Camelina sativa
MGAGGRMPVPSSSSKKSETDAIKRVPCEKPPFTLGELKKAIPPQCFKRSIPRSFSYLITDIIVASCFYYVATNYFSLLPQPLSYLAWPLYWACQGCVLTGVWVIAHECGHHAFSDYQWLDDTVGLIFHSFLLVPYFSWKYSHRRHHSNTGSLERDEVFVPKQKSAIKWYGKYLNNPPGRIMMLTVQFVLGWPLYLAFNVSGRPYDGFACHFFPNAPIYNDRERLQIYLSDAGILAVCFGLYRYAAAQGMASMICLYGVPLLIVNAFLVLITYLQHTHPALPHYDSSEWDWLRGALATVDRDYGILNKVFHNITDTHVAHHLFSTMPHYNAMEATKAIKPILGDYYQFDGTPWYVAMYREAKECIYVEPDREGDKKGVYWYNNKL

SEQ ID NO: 33 - protein, 384 aa, Camelina sativa
MGAGGRMPVPSSSSKKSETDAIKRVPCEKPPFTLGELKKAIPPQCFKRSIPRSFSYLITDIIVASCFYYVATNYFSLLPQPLSYLAWPLYWACQGCVLTGVWVIAHECGHHAFSDYQWLDDTVGLIFHSFLLVPYFSWKYSHRRHHSNTGSLERDEVFVPKQKSAIKWYGKYLNNPAGRIMMLTVQFVLGWPLYLAFNVSGRPYDGFACHFFPNAPIYNDRERLQIYLSDAGILAVCFGLYRYAAAQGLASMICLYGVPLLIVNAFLVLITYLQHTHPALPHYDSSEWDWLRGALATVDRDYGILNKVFHNITDTHVAHHLFSTMPHYNAMEATKAIKPILGDYYQFDGTPWYVAMYREAKECIYVEPDREGDKKGVYWYNNKL

SEQ ID NO: 34 - protein, 383 aa, Arabidopsis thaliana
MGAGGRMPVPTSSKKSETDTTKRVPCEKPPFSVGDLKKAIPPHCFKRSIPRSFSYLISDIIIASCFYYVATNYFSLLPQPLSYLAWPLYWACQGCVLTGIWVIAHECGHHAFSDYQWLDDTVGLIFHSFLLVPYFSWKYSHRRHHSNIGSLERDEVFVPKCKSAIKWYGKYLNNPLGRIMMLTVQFVLGWPLYLAFNVSGRPYDGFACHFFPNAPIYNDRERLQIYLSDAGILAVCFGLYRYAAACGMASMICLYGVPLLIVNAFLVLITYLCHTHPSLPHYDSSEWDWLRGALATVDRDYGILNKVFHNITDTHVAHHLFSTMPHYNAMEATKAIKPILGDYYQFDGTPWYVAMYREAKECIYVEPDREGDKKGVYWYNNKL

SEQ ID NO: 35 - protein, 387 aa, Camelina sativa
MVVAMDKRSNVNGDPGAGDQKKKGEGFDPSAQPPFKIGDIRAAIPKHCWVKSPLRSMSYVVRDICAVAALAVAAVYFNSWFLWPLYWAACGTLFWAIFVLGHDCGHGSFSDIPLLNSVVGHILHSFILVPYHGWRISHRTHHQNHGHVENDESWVPLPERVYKNLPHSTRMLRYTVPLPMLAYPLYLCYRSPGKEGSHFNPYSSLFAPSERKLIATSTTCWSIMFVTLIALSFIFGPLSFLKVYGVPYIIFVMWLDAVTYLHHHGHDEKLPWYRGKEWSYLRGGLTTIDRDYGIFNNIHHDIGTHVIHHLFPQIPHYHLVDATKAAKHVLGRYYREPQISGAIPIHLVESLVASIKKDEYVSDTGDIVFYETDPDLYVYASDKSKIN

SEQ ID NO: 36 - protein, 387 aa, Camelina sativa
MVVAMDKRSNVNGDPGAGDRNKKGEGFDPSAQPPFKIGDIRAAIPKHCWVKSPLRSMSYVVRDICAVAALAVAAVYFNSWFLWPLYWAACGTLFWAIFVLGHDCGHGSFSDIPLLNSVVGHILHSFILVPYHGWRISHRTHHQNHGHVENDESWVPLPERVYKNLPHSTRMLRYTVPLPMLAYPLYLCYRSPGKEGSHFNPYSSLFAPSERKLIATSTTCWSIMFVTLIALSFIFGPLSVLKVYGVPYIIFVMWLDAVTYLHHHGHDEKLPWYRGKEWSYLRGGLTTIDRDYGIFNNIHHDIGTHVIHHLFPQIPHYHLVDATKAAKHVLGRYYREPQTSGAIPIHLVESLVASIKKDHYVSDTGDIVFYETDPDLYVYASDKSKIN

SEQ ID NO: 37 - protein, 386 aa, Camelina sativa
MVVAMDKRSNVNGDPGAGDRNKKGEGFDPSAQPPFKIGDIRAAIPKHCWVKSPLRSMSYVVRDICAVAALAVAAVYFNSWFLWPLYWAACGTLYWAIFVLGHDCGHGSFSDIPLLNSVVGHILHSFILVPYHGWRISHRTHHQNHGHVENDESWVPLPERVYKNLPHSTRMLRYTVPLPMLAYPLYLCYRSPGKEGSHFNPYSSLFAPSERKLIATSTTCWSIMFVTLIALSFIFGPLSVLKVYGVPYIIFVMWLDAVTYLHHHGHDEKLPWYRGKEWSYLRGGLTTIDRDYGIFNNIHHDIGTHVIHHLFPQIPHYHLVDATKSAKHVLGRYYREPQTSGAIPIHVESLVASIKKDHYVSDIGDIVFYETDPDLYVYASDKSKIN

SEQ ID NO: 38 - protein, 387 aa, Artificial sequence
  Camelina sativa mutant
MVVAMDWRSNVNGDPGAGDRNKKGEGFDPSAQPPFKIGDIRAAIPKHCWVKSPLRSMSYVVRDICAVAALAVAAVYFNSWFLWPLYWAACGTLYWAIFVLSHDCGHGSFSDIPLLNSVVGHILHSFILVPYHGWRISHRTHHQNHGKVENDESWVPLPERVYKNLPHSTRMLRYTVPLPMLAYPLYLCYRSPGKEGSHFNPYSSLFAPSERKLIATSTTCWSIMFVTLIALSFIFGPLSVLKVYGVPYIIFVMWLDAVTYLHHHGHDEKLPWYRGKEWSYLRGGLTTIDRDYGIFNNIHHDIGTHVIHHLFPQIPHYHLVDATKSAKHVLGRYYREPQTSGAIPIHLVESLVASIKKDHYVSDTGDIVFYETDPDLYVYASDKSKIN

SEQ ID NO: 39 - protein, 386 aa, Arabidopsis thaliana
MVVAMDQRTNVNGDPGAGDRKKEERFDPSAQPPFKIGDIRAAIPKHCWVKSPLRSMSYVVRDIIAVAALAIAAVYVDSWFLWPLYWAACGTLFWAIFVLGHDCGHGSFSDIPLLNSVVGHILHSFILVPYHGWRISHRTHHQNHGHVENDESWVPLPERVYKKLPHSTRMLRYTVPLPMLAYPLYLCYRSPGKEGSHFNPYSSLFAPSERKLIATSTTCWSIMFVSLIALSFVFGPLAVLKVYGVPYIIFVMWLDAVTYLHHHGHDEKLPWYRGKEWSYLRGGLTTIDRDYGIFNNIHHDIGTHVIHHLFPQIPHYHLVDATKAAKHVLGRYYREPKTSGAIPIHLVESLVASIKKDHYVSDTGDIVFYETDPDLYVYASDKSKIN

SEQ ID NO: 40 - DNA, 41 bp, Camelina sativa
ctcatacaagaagatgaatcctttttgtgtgccttttgcaa

SEQ ID NO: 41 - DNA, 41 bp, Camelina sativa
ctcatacaagaagatgaatcctttttgtgtaccttttgcga

SEQ ID NO: 42 - DNA, 41 bp, Artificial sequence
  Camelina sativa mutant
ctcatacaagaagatgaatcttttttgtgtaccttttgcga

SEQ ID NO: 43 - DNA, 41 bp, Arabidopsis thaliana
ctcttacaagaagatgaatcctttttgtgtaccttttgcga

SEQ ID NO: 44 - protein, 553 aa, Camelina sativa
MVVGASSCYASPLCTWFVAACMSVSHDGGGGDSRQAAVALQSGGRSRRRRQLTKCSSASSPPRSPSPPTIQALVSSCLDFGPCTHYSNTNNNALSSLFGSNTLSVNRNQRRLNRATPSGGAMAVMEMEKEAMVNKKPPTEQRRVVVTGMGVETPLGHDPHTFYENLLQGNSGISQIENFDCSEFPTRIAGEIKSFSTEGWVAPKLSKRMDKFMLYLLTAGKKALADGGVTEEVMAEFDKTKCGVLIGSAMGGMKVFYDAIEALRISYKKMNPFCVPFATTNMGSAMLAMDLGWMGPNYSISTACATSNFCILNSANHIIKGEADVMLCGGSDAVIIPIGLGGFVACRALSQRNNDPTKASRPWDSNRDGFVMGEGAGVLLLEELEHAKKRGATIYAEFLGGSFTCDAYHMTEPHPDGAGVILCIERALASAGVSREQINYINAHATSTHAGDLREYQALVNCFGQNPELKVNSTKSMIGHLLGAAGAVEAVATIQAIRTGWVHPNINLENPDSGVDTKLMVGPKKERLNIKAALSNSFGFGGHNSSIIFAPYK

SEQ ID NO: 45 - protein, 585 aa, Camelina sativa
MVVGASSCYASPLCTWFVAACMSVSHEGGDSRQAAVALQSGGRSRRRRQLTKCSSASSPPRSPSPPPTIQALVSSCLDFGPCTHYSNSNSNNNALSSLFGSNPLSLNRHQRRLNRAASPSSGGAMAVMEMEKEAMVNKKPPTEQRRVVVTGMGVETPLGHDPHTFYENLLQGNSGISQIENFDCSEFPTRIAGEIKSFSTEGWVAPKLSKRMDKFMLYLLTAGKKALADGGVTEEVMAEFDKTKCGVLIGSAMGGMKVFYDAIEALRISYKKMNPFCVPFATTNMGSAMLAMDLGWMGPNYSISTACATSNFCILNSANHIIKGEADVMLCGGSDAVIIPIGAWSFSYITLFPLFLIFKPAKSNRMCSNAGLGGFVACRALSQRNNDPTKASRPWDSNRDGFVMGEGAGVLLLEELEHAKKRGATIYAEFLGGSFTCDAYHMTEPHPDGAGVILCIERALASAGVSREQINYINAHATSTHAGDLREYQALVNCFGQNPELKVNSTKSMIGHLLGAAGAVEAVATIQAIRTGWVHPNINLENPDSGVDTKLMVGPKKERLNIKAALSNSFGFGGHNSSIIFAPYK

SEQ ID NO: 46 - protein, 550 aa, Camelina sativa
MVVGASSCYASPLCTWFVAACMSVSHDGGGDSRQAAVALQSGGRSRRRRQLTKCSASASRSPSPPTIQALVSSCLDFGPCTHYSNSNNNALSSLFGSSTLSLNRHQRRLNRATSSGGAMAVMEMEKEAMVNKKPPTEQRRVVVTGMGVETPLGHDPHTFYENLLQGNSGISQIENFDCSEFPTRIAGEIKSFSTEGWVAPKLSKRMDKFMLYLLTAGKKALADGGVTEEVMAEFDKTKCGVLIGSAMGGMKVFYDAIEALRISYKKMNPFCVPFATTNMGSAMLAMDLGWMGPNYSISTACATSNFCILNSANHIIKGEADVMLCGGSDAVIIPIGLGGFVACRALSQRNNDPTKASRPWDSNRDGFVMGEGAGVLLLEELEHAKKRGATIYAEFLGGSFTCDAYHMTEPHPDGAGVILCIERALASAGVSREQINYINAHATSTHAGDLREYQALVNCFGQNPELKVNSTKSMIGHLLGAAGAVEAVATIQAIRTGWVHPNINLENPDSGVDTKLMVGPKKERLNIKAALSNSFGFGGHNSSIIFAPYK

SEQ ID NO: 47 - protein, 550 aa, Artificial sequence
  Camelina sativa mutant
MVVGASSCYASPLCTWFVAACMSVSHDGGGDSRQAAVALQSGGRSRRRRQLTKCSASASRSPSPPTIQALVSSCLDFGPCTHYSNSNNNALSSLFGSSTLSLNRHQRRLNRATSSGGAMAVMEMEKEAMVNKKPPTEQRRVVVTGMGVETPLGHDPHTFYENLLQGNSGISQIENFDCSEFPTRIAGEIKSFSTEGWVAPKLSKRMDKFMLYLLTAGKKALADGGVTEEVMAEFDKTKCGVLIGSAMGGMKVFYDAIEALRISYKKMNLFCVPFATTNMGSAMLAMDLGWMGPNYSISTACATSNFCILNSANHIIKGEADVMLCGGSDAVIIPIGLGGFVACRALSQRNNDPTKASRPWDSNRDGFVMGEGAGVLLLEELEHAKKRGATIYAEFLGGSFTCDAYHMTEPHPDGAGVILCIERALASAGVSREQINYINAHATSTHAGDLREYQALVNCFGQNPELKVNSTKSMIGHLLGAAGAVEAVATIQAIRTGWVHPNINLENPDSGVDTKLMVGPKKERLNIKAALSNSFGFGGHNSSIIFAPYK

SEQ ID NO: 48 - protein, 541 aa, Arabidopsis thaliana
MVGASSSYASPLCTWFVAACMSVSHGGGDSRQAVALQSGGRSRRRRQLSKCSVASGSASIQALVTSCLDFGPCTHYNNNNALSSLFGSNSVSLNRNQRRLNRAASSGGAMAVMEMEKEAAVNKKPPTEQRRVVVTGMGVETSLGHDPHTFYENLLQGNSGISQIENFDCSEFPTRIAGEIKSFSTEGWVAPKLSKRMDKFMLYLLTAGKKALADGGVTDEVMAEFDKTKCGVLIGSAMGGMKVFYDAIEALRISYKKMNPFCVPFATTNMGSAMLAMDLGWMGPNYSISTACATSNFCILNSANHIIKGEADVMLCGGSDAVIIPIGLGGFVACRALSQRNNDPTKASRPWDTNRDGFVMGEGAGVLLLEELEHAKKRGATIYAEFLGGSFTCDAYHMTEPHPDGAGVILCIERALASAGISKEQINYINAHATSTHAGDIKEYQALAHCFGQNPELKVNSTKSMIGHLLGAAGAVEAVATVQAIRTGWVHPNINLENPDSGVDTKLLVGPKKERLDIKAALSNSFGFGGHNSSIIFAPYK

SEQ ID NO: 49 - DNA, 33 bp, Artificial sequence
  primer
gagcggccgcatcacagatcgattctctcttaa

SEQ ID NO: 50 - DNA, 31 bp, Artificial sequence
  primer
atgcggccgcgaacaatgtatgtaatgtacc

SEQ ID NO: 51 - DNA, 21 bp, Artificial sequence
  primer
atgggtgcaggtggaagaatg

SEQ ID NO: 52 - DNA, 22 bp, Artificial sequence
  primer
cctcataacttattgttgtacc

SEQ ID NO: 53 - DNA, 23 bp, Artificial sequence
  primer
atggttgttgctatggacaaacg

SEQ ID NO: 54 - DNA, 28 bp, Artificial sequence
  primer
ttaattgattttagacttgtcagaagcg

SEQ ID NO: 55 - DNA, 21 bp, Artificial sequence
  primer
atgacgtccgttaacgcaaag

SEQ ID NO: 56 - DNA, 21 bp, Artificial sequence
  primer
ttaggaccgaccgtttttgac

SEQ ID NO: 57 - DNA, 30 bp, Artificial sequence
  primer
atctgagaatctcatacaagaagatagatc

SEQ ID NO: 58 - DNA, 27 bp, Artificial sequence
  primer
acatttgaaatttaacagaccagatcc

SEQ ID NO: 59 - DNA, 24 bp, Artificial sequence
  primer
aaccaccgttcacgctgggatatc

SEQ ID NO: 60 - DNA, 22 bp, Artificial sequence
  primer
cctcataacttattgttgtacc

SEQ ID NO: 61 - DNA, 25 bp, Artificial sequence
  primer
cctttactgggccatcttcgtctgc

SEQ ID NO: 62 - DNA, 25 bp, Artificial sequence
  primer
catagtggaacgttactgaaaagtg

SEQ ID NO: 63 - DNA, 29 bp, Artificial sequence
  primer
ttattgcagttacccccttataggtttgg

SEQ ID NO: 64 - DNA, 28 bp, Artificial sequence
  primer
cgatctcctggcttgttggagagcaaaa

SEQ ID NO: 65 - DNA, 25 bp, Artificial sequence
  primer
gatggtcgttaacactttcaggatc

SEQ ID NO: 66 - DNA, 21 bp, Artificial sequence
  primer
ggtatgagtccgaaccgtgtg

SEQ ID NO: 67 - DNA, 2791 bp, Camelina sativa
gcggaggagcttcttcctcgtagggggtttcttcttcttcatcgtttttaacgaaccatcgttaagtcaaatctccccccccccctacgtcagctccaggtccgtccttctcatttccgatttcgattcatttacgtctcgtctggtctgttctgtgtttttttttttctttttctttctcccgcactatctcatttccgatgtttttttaaataaaaaccgatttcattatatagatctggcttatatgtcttgcattcaaccttagatctggtctcgatgctctgtttttttctttagttgagaaatctgatgttgttacaatgagttcttattcatataatgattactagtgccttgggtcatccatgaaaacgatatgttaatgctatgatttttttatttgttttctttgtcaaaaatgaagtgctgctttgacccattcttctttagatatttttattttatttttgttgggttggtagaatagtgagaatcaccataaaattctcttatcagtttcacgtccctggttttttttattttttatttatttaaagatctgtaatatattcagttttccctaattttgtttgtgtaaaatttgctttgaattgccgtgttgaatctcttatggatttgacctatgcctaccgggtcttaagattgatgataaaactttttaaatagacaaaaaaaaaagtttcactgattgattctcataaactttacaatgaagttggaattagggtaattcaggatcagatgcgtagtagattcagatgcaaaataatgagttgcatgacttgttaatattatagatccataaagacatatttaaatatctgacatatgatgtcggcaaaattcggtggtatatagtatacatcacttagaaactgtttcctttggacttgtttgccaacttggttgtattcaggaggatttgtgattttgattgatccatttactttctctattgttttttctttttcttttggggtctacttggtggtattcataagagaacttttgtttttgattgaatttaattacaagaaactgatgatgataaccacaataaagagattgtgacctgtcgtattgaaatcttattagtagtagcagtcgtgttctcaacgtcaatgggtttcctttctttggtttcttactttacgccgcttctctgctctttttgttcgttttggtccacgcactttccttttttgtggcaatccctttcacaacctcatctctgaataataataattattactagtttgttgatttgatcattaccacctcgttttctagtgcatgcaaaatttgtcaattagtgtgataaacaaacaattcctttcttgagtttcagctttttgatttttctttgctctatgtttcttttgcagaatcatgggtgcaggtggaagaatgccagttccttcttcttcttccaagaaatctgaaaccgatgccataaagcgtgtgccctgcgagaaaccaccgttcacgctgggagatctgaagaaagcaatcccaccgcagtgtttcaaacgctctatccctcgctctttctcctaccttatcactgacatcattattgcctcctgcttctactacgtcgccaccaattacttttctctcctccctcagcctctctcttacttggcttggcccctctattgggcttgtcaaggctgtgtcctaaccggtgtctgggtcatagcccacgaatgcggtcaccacgcattcagcgactaccagtggctcgatgacacagtcggtcttatcttccattccttccttctcgtcccttacttctcctggaagtacagtcatcgccgtcaccattccaacacaggatccctcgaaagagatgaagtatttgtcccaaagcagaagtccgctatcaagtggtatggcaaatacctcaacaaccctgctggacgcatcatgatgttgaccgtccagtttgtcctcgggtggcccttgtacttggcctttaacgtctccggcagaccatacgacgggttcgcttgccatttcttccccaacgctcccatctacaacgaccgtgaacgcctccagatatatctctctgatgccggtattctagcagtctgttttgggctttaccgttacgccgctgcacaaggattggcctcgatgatctgcctctacggagtaccacttctgatagtgaacgcgttcctcgtcttgatcacttacttgcagcacactcatcctgcgttgcctcactacgattcatccgagtgggattggcttaggggagctttggctaccgtagacagagactatggaatcttgaataaggtgttccacaacatcacggacacacatgtggctcatcatctgttctcgacaatgccgcattataatgcgatggaagctacaaaggcgataaagccaatactcggtgactattaccagttcgacggaacaccatggtatgtggccatgtatagggaggcaaaggagtgtatctatgtagaaccggacagggaaggtgacaagaaaggtgtgtactggtacaacaataagttatgaggatgatggtgaaagaacactgaagaaattgtcgatctttctctagtctggttctcttttgtttaagaagttatgttttgtttcaataatttcagtgtccattttgttgtgttatgacattttggcaaattatgtgatgtgggaagttagtgttcaaatgttttgtgtctgtattgttcttctcatcgctgttttgttgggatcgtagaaatgtgaccttcgg

SEQ ID NO: 68 - DNA, 3875 bp, Camelina sativa
  misc_feature (3864)..(3864): n is a, c, g, or t
  misc_feature (3867)..(3867): n is a, c, g, or t
gtggaggagcttcttcctcgtagggggtttcttcttcttcatcgttattaacgaaccatcgttgagtcaaatctcccaaccccctacgtcagctccaggtccgtcctttctcatttcatattccgattcatttacgtctcgtctgatctgttctttgtttttattttttttttctttctcccgcactatctcattttcgattctttttttttttagaaccgatttgatgatatagatctggcttatatgtcttgcattcaaccttagatctggtctcgatgctctgttttttctttagttgagaaatctgatgttgttgttacaatgagttcttattcataataatgattactagtgccttgggtcatccatgaaaacgatatgttgttatactatgattttttatttgtcaaaaatgaagtgctgctttgacccattctctttagatttattatttttgttgggttggtagaatagtgagaatcaccataaaattctcttatcagtttcacgtcctgtttttttttcaaaaagatccgtaatatattcagtttttttttatttgtgtgtaaaatttgctttgtattgccgtgttgaatctcttatggatttgacctatgcctaccgggtcttatggattgatgatataactttttaaacagacaaaataagtttcactgaatgattctcataaactatacaataaagttggaattagggtaattcaggatcagatgcgtagattcagatgcaaataatgagttgcatgacattttattattatagatccgtaaccgtaaagacatattatgttctgttttaaatatctgatatatgatgtcggcaaatttcggtggtttatacatcacttaaaaactgtttcctttggacttgtttgccaacttggtgctattcaggaggatttctgattttgattgatccatttactttctctattgttttttttttttttgggggtctacttgttggtattcataaaagaatttttgatcttgattgaatttaattacaagaaactgctgatgataaccacaataaagagattgtgacctgtcgtattgaaatcttattagtagtagtagtcgtgttctcaacgtcaatgggtttttctttctttggtttcttactttacgccgcttctctgctctttttattccttttggtccacgctctctccttttgtggcaatccctttcacaacctcatctctgaataacaataattattactagtttgttgatttcatcattaccactcgttttctagtgcatgcaaaatttgtcaattagtgataaactgaaaattcctttcttgatttttctttgctcttggtttgttgcagaatcatgggtgcaggtggaagaatgccagttccttcttcttcttccaagaaatcagaaaccgatgccataaagcgtgtgccctgcgagaaaccaccgttcacgttgggagaattgaagaaagcaatcccaccgcagtgtttcaaacgctctatccctcgctctttctcctaccttatcactgacatcattgttgcctcctgcttctactacgtcgccaccaattacttctctctcctccctcagcctctctcttacttggcttggcctctctactgggcttgtcaaggctgcgtcctaaccggtgtctgggtcatagctcacgaatgcggtcaccacgcattcagcgactaccaatggcttgatgacacagttggtcttatcttccattccttccttctcgtcccttacttctcctggaagtacagtcatcgccgtcaccattccaacacaggatctctcgaaagagatgaagtatttgtcccaaagcagaaatcagctatcaagtggtatggcaaatacctcaacaaccctcctggacgcatcatgatgttaaccgtccagtttgtcctcgggtggcccttgtacttggcctttaacgtctcgggcagaccgtacgacgggttcgcttgccatttcttccccaacgctcccatctacaacgaccgtgaacgcctccagatatatctctcggatgccggtattctagcagtctgttttgggctttaccgttacgctgctgcacaaggaatggcctcgatgatctgcctctacggagtaccgcttctgatagtgaacgcgttcctcgtcttgatcacttacttgcagcacactcatcctgcgttgcctcactacgattcatccgagtgggattggcttaggggagctttggctaccgttgacagagactatggaatcttgaacaaggtgttccacaacatcacggacacacatgtggctcatcatctgttctcgacaatgccacattataatgcgatggaagctacaaaggcgataaagccaatactcggtgactactaccagttcgacggaacaccgtggtatgtggcgatgtatagggaggcaaaggagtgtatctatgtagaaccggacagagaaggtgacaagaaaggtgtgtactggtacaacaataagttatgaggatgatgatggtgaaagaacactgaagaaattgtcgaactttctctagtctggttctcttttgtttaagaagttatgttttgtttcaataatttcaatgtccattttgttgtgttatgacattttggcaaattatgtgatgtgggaagttagtgtttaaatgttttgtgtctgtattgttcttctcatcgctgttttgctgggatcgtagaaatgtgaccttcggacagtaaaactctactaaaactatcttcctttcggtatcttcaaaagtgttaacttaactatgatgcacgtagtgaatcctgacttaaataatcgacttctgtttaagacctatcaactgtaagagggttacacgaatgtttctttaacaaaataaacataacaattgctctctctaaattaggttcgatgtttttgtctgtttgtttgatgcatggtagtcggagtagctgtcatgttcaagttcaatcttcagtttagaacttgtttccattgttttatgactagcactgaattccattgtactctctgtctgtgatactgaagccaagcgtgacaaatgttgaacatgccatgtcgatgtattaaaggggattgagttaatagtgctgttttggctggcaggtcacaatcacaattctttcacactccaatcatgtggttaggcttacgattccttttttttagaattagcttttggtaaagaactgagacctctggcctttacatatgaaatatgaaacccctttaactaaaattatatagcacgccaaatccattacctctggtttcatctttgagagggaacattaagagtaaaagaaagagaaataaataaaaaataattttttccattacagaatcaccaaaagagaaggacaacaagaaagaaatgaggtgaagaaacatagaaaacaaaagaatgttctgtaaccaagtcgatcgatgaacaaaaggctttaccaatacggaaacaatctttcatcccttcgatttaagcataaacttagaagcatttcctgtggactatggatggccctgactcatcatactcaccctttgatatccacatctgtaaagcaacaacattgtgtatgattaacaaatttcaaatgggtaacaaagtaagtaaaaaagcacaaaaactcatagagaataaagaatgaagatattacctgttggaaggtactgagggaagctaaaatggagcctccaatccagacactgtacttcctctcaggtggtgcaaccaccttaatcttcatgctgcttggagccaaagcagtgatctctttgctcattctatcagcaattccagggaacatggtggttccancantgagcaag

SEQ ID NO: 69 - DNA, 2725 bp, Camelina sativa
gcggaggaccttcttcctcgtagggggtttcttcttcttcatcgttattaacgaaccatcgttaagtcaaatctcccccccccccccctacgtcagctccaggtccgtccttctcatttccgatttcgattctttaacgtatcgtctggtctgttctgtgtttttatttttttctttctttctcccgcactatctcattttcgatttttttttaataaaaaaaaaaccgatttcgtgatatagatctggcttatatgtcttgcattcaaccttagatctggtctcgatgctctgttttgtttttttttttttgttgagaaatctgatgttgttacaatgagttcttattcatataatgattactagtgccttgggtcatccatgataacgatatgttatactatgatttttattttatttttcttttgtcaaaaatgaagtgctgctttgacccattctctttagatatttttatttttttatttttgttgggttggtagaatagtgagaatcaccataaaattctcttatcagtttcacgtcctggctttttttcttgtttttgtttttttttaaagatctgtaatatattcagttttccctatttttgtttttgtaaaatttgctttgaattgccgtgttgaatctctcttatggatttgacctatgcctaccgaggtcttatggattgatgatatgactttttaaataaagttggaattagggtaattcaggatcagatgcgtagtagattcagatgcaaataatgagttgcatgacttgaaaatattatagatccgtaaagacatatttaaatatctgacatatgatgtcggcaaatttcggtggtttatacatcactcaaaacttaaaactgtttcttttggacttgtttggcaacttggtggtattcaagaggatttgtgattttgattgatccatttactttctctattgttttttcttttggggtctacttgttggtattcataagagaactttgtgatcttgattaaatttaattacaagaaactgatgatgatatccacaataaagagattgtgacctgtcgtattgaatatcttattagtagtagtagtcgtgttctcaacgtcaatgggtttctttctttggtttcttactttacgccgcttctctgctctttttattccttttggtccacgcactttccttttgtggcaatccctttcacaacctaatcttcaatttggatcatttctctgaataataataacaactagtttgttgatttgatcactaccactcgttttctagtccatgcaaaatttgtcaattcctttattcctttgatttttttgcagaaacatgggtgcaggtggaagaatgccggttccttcttcttcttccaagaaatcagaaaccgatgccataaagcgtgtgccttgcgagaaaccgccgttcacactgggagaattgaagaaagcgatcccaccgcagtgtttcaaacgctctatccctcgctctttctcctaccttatcactgacatcattgttgcctcctgcttctactacgtcgccaccaattacttctctctcctccctcagcctctctcttacttggcttggcccctctattgggcttgtcaaggctgtgtcctaaccggtgtctgggtcatagcccacgaatgcggtcaccacgcattcagcgactaccaatggcttgatgacacagttggtcttatcttccattccttccttctcgtcccttacttctcctggaagtacagtcatcgccgtcaccattccaacacaggatctctcgaaagagatgaagtatttgtcccaaagcagaagtccgctatcaagtggtatggcaaatacctcaacaaccctgctggacgcatcatgatgttaaccgtccagtttgtcctcgggtggcccttgtacttggcctttaacgtctcgggcagaccatacgatgggttcgcttgccatttcttccccaacgctcccatctacaacgaccgtgaacgcctccagatatatctctctgatgccggtattctagcagtctgttttgggctttaccgttacgccgctgcacaaggattggcctcgatgatctgcctctacggagtaccacttctgatagtaaacgcgttcctcgtcttgatcacttacttgcagcacactcatcctgcgttgcctcactacgattcatccgagtgggattggcttaggggagctttggctaccgtagacagagactatggaatcttgaacaaggtgttccacaacatcacggacacacatgtggctcatcatctgttttcgacaatgccgcattataatgcgatggaagctacaaaggcgataaagccaatactcggtgactattaccagttcgacggaacaccatggtatgtggccatgtatagggaggcaaaggagtgtatctatgtagaaccggacagggaaggtgacaagaaaggtgtgtactggtacaacaataagttatgaggatgatggtgaaagaacactgaagaaattgtcgatctttctctagtctggttctcttttgtttaagaagttatgttttatttcaataattccagtgtccattttgttgtgttatgacattttggcaaattatgtgatgtgggaagttagtgttcaaatgttttgtgtctgtattgttcttctcatcgctgttttgttgggatcgtagaaatgtgaccttcgg

SEQ ID NO: 70 - DNA, 5288 bp, Camelina sativa
ggtatgaattggcttacacggaagccaaaggaagaatgaggaaagggaacagagtttggcagattgcttttggaagcgggtttaagtgtaacagcgcggtttgggtggctctccgcgatgtcaagccctcggttaacaatccttgggaacattgcatccatagatatccagttaagatcgatctttgaactcgtaagaaccggtccgaaaacatggttagtccccctccatgtaccaaaaaaaaaaagtttaactcttatatttttagttctttaccaatgggtcaagaaattctgttgaaggtaacacttaaatgtatgtatgtgtttattatattatttattataattaaaaaaatagttttatttccggatataccataagttgaatttttaaaaacaaatataaattgttcaatctataaaatattcaagctttagtaatattattctttaaaaataatatctattgaattaaaaaatttactaagtaacgggtcaaaatttgaatatttaaattcaatttcatattttttgttaaattttataattttatataatataataaactttaaataatttattttgaaatatttttaatatattgaaacttgatataaaagttagaaactataaacactctattgaaataaaagttatcaataatatttcactataatatgaaagaatttcaagaaaccaagtatattaaaacaaaacgtataaaaatatattaaattactcataatataatataactcactttttaaaaaccaaatttacaaaattatgttttataatgacattcaagtcatgatgtagaatatacattgttgaaataatttcacatacataaactaatataacatattaaaattttattttaaaatataaaatatacataattttgtataaataaaatttaaccagtgttatagcacgggtaattatctagaatcattaacaatataaaacttataaaataagttttttttttcaagctatcatatttagcatatgattttattgcataatgttttatccaaaaaacttttaaaaacaagtatataaattacattttatataaaaattacaatataaataaaataaatttcaacacgtactctaacacggatcttaatctagtatgttgtgtataaaggtaacactaaaatgaccaagaatggttacgaagtcaaaagatgggaccaaaagcgttgacaaaattttagttcttttctaaaaataaaattgtttgtataataaaaattgttaggtagaacttagaacactcaactaatatatccttgcgtacgaaaacatgtgttaagtgaagtgaccttaatgtagtggccaaaagtaagtctttaatgcacaagacaccatcacaccagagatcgagtctcgttccctacgaatgtagggattagtgtaatggtccgacaaaaaaaacaaaacatgtattaaatttagaatacatgaaattactcacaagttgcgaatacatgtacccctcggctctgggatgctaaaccgggttcgtcttatactcaaatataacatttaagtttgaaatttttttttgtcaataaaaataatttgtatactaaaaaatttagttaaaagtttaaaacaagttttacttaaaaaaaaatatttgtgaaaaaatccgaagtgttaaatttagaacacacaaaaatatgttggacttatgacacgaagcttacagatccgaaatgtttagtcagattaatcttcaagcgtatttaagtttaacacttaagtttgtaaatatacgaaaatattggttaaaaaaccttaaattttagtatcgacaaaaaaaaaatcttaaattttagtttaaaatctgaaaagaaaaagtgaaaacctttttgtttatttgtaggaaaaatcaatcacttaaaactaaaaaaaactagaaaaagaaataaagaaagcaatatacctctgcttgtgatatggaagtggaagaaatacaagatttgatcgattctcatcatcttgagatgactgaatagtcggaaccatggaaacaccaaacacacaggatcatgactggttctctactctcactcgtcctcgttcctgcattctcgcatccttgagccgtcgttgatatatccaccgtgtgaaaacgtttcggtgatcgtagagaaaattttgacctaatttaaatcgatgacgatcggatgaagatgatgtagtcacgatttaacaagagagattaatcacgaaaagagatgctctccgctccgatcaaatatcaaaaagagatcatctctttttgtgattgtgtaagaagttagagagaagacttgacgtcctgtaatcgtgatctcgatcgaatgaagatatgtcgtcacgattaacatgaggatggcgatcaatcaatcacgaacagagtcagagatgcttcccctaatcttgacagagatcaccaacgcaaagctttggatttggtttctcaaagaagaagaagaagagagatttggaaaactttgacaaagaagaaaatgagagaatgaatgaatgatgtgatgttggagattttttaacctaatgactaaatgagccgtcttttatatacgcaacagctatattaatatttttttattttttattttctaatttcagcaaatttaaataacgaaattgtgattgtttcctttgttttcttttccccacgagttctcaagttgtttgttttcttattttggttgcaaccaaaaaaaacaagacagaaaaaacaatttatctcaagagtaacaaaaaggagattcgagatcctttggagttactcaattacattcatatgtcacgagataaaaaggttaaacaatcactggatcagtgtgctcatggtgttccaggtccaaaacatgtgtcacgagataaaaacaaagagaaacacaactgaaaatgattaccaagatcacaactatatataaaggacttataaaaaatgaatttgaaagtggttaaactaagtgattataagtgttattgcagttacccccttataggtttggtgaatcttattagagataacttattcttaagatagttgcaattaaccaaaaaaaaaaattgtccggatagtttgatgcaattaaatgattaatgagtgttctatagggtctgattcttaatatttcgaaatatttggccttaactaaacttccaccatgatttatttactgatctagttcggggacagactttgcgaataaaactcattaccgagaaacattcatcccataattgctatttagtcagaggctaatcgactatggcctttcagccaatcaaagctacgaacacgaatctccctaaaacatcctcaagtattttatttaatacacatgtatcgtattgagcaccactcataaactaatttcatacatttatcatactctttatttgtaataataaaagcatcaacatattgtaggcaattagaatcaaaacaaaacattttttttttctttccaaattttcaaaattggtaaacgaaacttggacctttaatacttatattggcaatataataatattgcagagtggactatttcccttattttggcaactttcagtggactagtaatttatttcaatgtggatgcttgcatgagtgtgaatatacacatgtctatatgcatgcctgcaaatcgtaacggaccacaaaaaaggatccatacaaatacctcttaacggctcctctctatcatactctccgacacaaactgagcaatgacgtccgttaacgcaaagctcctttaccattacgtcctaaccaactttttcaacctttgcttgtttccgttaacggcgttacttgccggaaaagcctctaggcttacctcaaacgatctctaccacttctattcccatctccaacacaaccttataaccgtaattttactctttgctttcaccgctttcggtttggttctctacattgtaacccggcccaaaccggtttacctcgttgactactcgtgctaccttccaccaccgcatctcaaagttagtgtttccaaggcgatggatattttctaccaaataagaaaagctgatacctcacggaacgtggcatgcgatgatccatcctcgcttgatttcctgaggaagattcaagaacgttcaggtctaggtgatgaaacctacagtccccagggactcattaacgtgcccccacgaaagacctttgcagcttcacgtgaagagacagagcaggtaatcatcggtgcgctagataagctattcgagaataccaaagttaaccctagagaaattggtatacttgtggtcaactcaagcatgtttaatccaactccttcgctatctgcgatggtcgttaatactttcaagcttcgaagcaacatcaaaagctttagtctcggaggaatgggttgtagtgctggtgtcatcgccattgatcttgcaaaggacttgttgcatgttcataaaaacacttatgcacttgtggtgagcactgagaacatcactcaaggcatttatgctggcgaaaatagatccatgatggttagcaattgcttgttccgtgttggtggcgcagcgattttgctctccaacaagccaggagatcggagacggtccaagtacaagttatgtcatactgttcgaacgcataccggagctgatgacatgtcttttcgatgtgtgcaacaaggagacgatgagagcggtaaaatcggagtttgtctgtcaaaggacataaccgttgttgcggggatagcgcttaagaaaaacatagcaacgttgggtccgttgattcttcctttaagcgaaaaatttctgtttttagtaaccttcatcgccaagaaacttttgaaggacaagatcaagcactattacgtcccggatttcaagcttgctattgaccatttctgtattcatgcgggaggcagagccgtgatcgatgtgcttgagaagagcttaggactatctccaatcgatgtggaggcatctagatcaacgttacacagatttgggaatacttcgtctagctcaatttggtatgaattggcatacatagaagcaaaaggaaggatgaagaaagggaatagagcttggcagattgctttagggtcaggatttaagtgtaacagtgcggtttgggtggctctatgcaatgtcaaggcttcggcgaatagtccttgggaacattgcatcgatagatatccggttcaaattgattctggttcatcaaaatcagatactcatgtcaaaaacggtcggtcctaa

SEQ ID NO: 71 - DNA, 5097 bp, Camelina sativa
  misc_feature (12)..(12): n is a, c, g, or t
ggtatgaattgncttacacggaagccaaaggaagaatgaggaaagggaacagagtttggcagattgcttttggaagcgggtttaaatgtaacagcgcggtttgggtggctctccgcgatgtcgagccctcgtttaaaaatccttgggaacattgcatcgatagatatccggttaagatcgatctttgaactcgtaagaacggtagattggtctggaaacatggttagtcctccatgtaccaaaaaaaaaaggttaactcttatatcttttgttctttaccaaggggtcaagaaaatcagtggaaggttaatgtatgtttgtatatgttgtgtataaaggtaacccttaattaatgaccaagaatggttatgaagtcaaaagataggaccaaaagtgttgacctattaaaatttaaaattttagttttttggaaaaattaaatcatttgtgaaataaaaatggtgagttagaaaaatcaaaacaatttacttacttacaaagaaccttaaaattaaacttaagtgttaaatttagaataaatgaaaaaatgctaaacttatgacaccaaaacctacgaactcttataaggacggggatactaaatttgtttgtttgtttgtttttttttttttttttgtaaatcaatccggtttgttttcaatgtattcaaattaaagtcgggttatgacaccaacaccaagcttacggatctgaggaagacgggatgctaagtcgggttagtcatcaagcgtattcaaataactctgaagtcttagaactttttttgttaataaaatattttgtataataaaaaaaatttagttaaaaaaatttaaaacaatttttacataaaatattatttgtgaaagaactttttaagtgttaaatttagaacactcgaaaacatgttagacttgtgacaccaagcttattccaatttaacgcttaagtttgaaaactttttttgtcaataaaaaatatactatatgaaaagattggttataaaaaccttaaatttagtttaaagtctgaaaataaaaataaaaaataaaaaaaattattagttggaaaggaaataaagaaagcaatatacctctggttgtgatgtggaagaaacacaagatttgatcgattctcatcagcttcagatgactgaatgaatagtcggaaccatggaaacaccaccaaacacacaggatcatgactggttcactactctcactcgtcctcgttcccgcattctcgcatccttgagccgtctttgacagatggttaaaacacgaacagagctgaataagtagaagaaaaaagagagagtttgagaagtttgatcgaatgaataagaagaaagactttttttgacctggaatcgatctcgattgaacagagatgttttcctctttaccgcattagatcagatcaatccaccgtgtgaaaacgtttcggtgatcgtagagcagattttgacctaattaattaagatcgatcacgtacgatcggatgacgataatgtaagagatcaatcacgaaaagagatgctccgatcaaatatcaaagggataataaagttgatgcagagatcatctctgttcgtggttgtttgagattagggagaagacttgacctggaatcaatctcgatcgaatgaagatgttgtcacgattaacatggggatggcgatcagtcaatcacgaacagagtctgatatgattccccctccgatcttgacagagatcaccaacgcaaagctttggatttggtttctcaaagaagaagaagaagagagatttggagaaactttgatggttttacaaagaagaagagagaatgaatgaatgatttggagattttttaacctaatgactaataataatgagccgagtgtatgaatgtatttaatataagcaacggctattttttcttcttttcttttaactttaagcaaatttagaaaaggaaattgtgattgtttccttttttccccacgagctctcaagttgaatccttttcttatttgagttgcaaccaataataaaaaaaaaagatagaaaaacggaaatttatcacaagagtaacaaaaaggagattcgagatcctttagagtttactcaattacattcatatgtgctagttgtgggagtgagaggagttttctccttccgaagtgatttatgtatggaggagtttatcaccgttaagagttccgaattgaaagagactatttgattgctaaaattgtatattactgtctgagagaaaaaatattcgatcccccacaaagtctccccccccttatatatttatacagaccaattaattacccaattaatgcgtaattaattttacacgattctcgcgtcctaattaattcacttgaatgctagaatctttgaccgaggtcgaactgcgagctgactaatatgacgcctctccgcgctctcttcttctctctgggcctgatgggccgaccagcgctacacctttgacccgtttagcgtgcccggcccaggtcctctgcctgttatccgagatgacgaatcgtgggtacaacactagtgtatgaccttttgcctttataggcactaaaaaactacccaaaaaaaatatgaagaagaaaaaaaggttaaacaatcactggatcagtgtgctcatggtgttccaggtccaaagcatgtgtgtcacgagataaaaatagagagaaacacaactgattatgatcaccaagatcacgaatatatataaaggacttataaaaaatgaatttgaaagtggttaaactaagtgattatatgtgtgattgctcttagccccttaggtgtggtgaatcttattatagagatgacttattttaaagatagttgcaattaaaaaaaaaaatcagtgtccggatagtttgatgcaattaattagtgttctatagggtctgattcttaatatttatgcaaatattatagtatttcaaagtatttggccttaactaaacttccacctgatttatttactgatctagttcggggacagactttgcgaataaaactcgttcccgagaaacattcatcccataactgctatttagtcagaggctaatcgactatagcctttcagccaatcaaatctacgaacacgaatccccctaaaacatcctcaagtatttatttaatacacatgtatcgtattgagcaccactcataaactatttttttttttgtttttaacaaaaaaaatttatcatactcttttgtaataatagatgcatcaacatattgtaggcaacgttgaagaaccagtacattctttttttttttgctccaaattttcaaaattggaaaatgaaacttggacgaaataaatttaacactctgtatatattggcaatataatattgcagagtggactatttaccttattttggcaactttcagtggactagtaatttatttcaatgtgtatgcttgcatgagtgtgaatatacacatgtctatatgcatgcttgcaaatcgtaacggaccacaaaaaaggatccatacaaatacctcttaacggctcctctctatcatactctccgacacaaactgagcaatgacgtccgttaacgcaaagctcctttaccattacgttctaaccaactttttcaacctttgcttgtttccgttaacggcgttacttgccggaaaagcctctaagcttacagcaaacgatctctaccacttctattcccatctccaacacaaccttataaccgtaattttactctttgctttcaccgctttcggtttggttctctacattgtaacccggcccaaaccggtttacctcgttgactactcgtgctaccttccaccaccgcatctcaaagttagtgtttccaaggcgatggatattttctaccaaataagaaaagctgatacctcacggaacgtggcatgcgatgatccatcctcgcttgatttcctgaggaagattcaagaacgttcaggtctaggtgatgaaacgtacagtccccagggactcattaacgtgcccccacaaaagacctttgcagcttcacgtgaagagacagagcaggtaatcatcggtgcgctagaaaagctattcgagaacaccaaagtaaaccctagagagattggtatacttgtggtgaactcaagcatgtttaatccaactccttcgctatctgcgatggtcgttaacactttcaagctccgaagcaacatcaaaagctttagtctcggaggaatgggttgtagtgctggtgttatcgccattgatcttgcaaaggacttgttgcatgttcataaaaacacttatgcacttgtggtgagcactgagaacatcactcaaggcatttatgctggcgaaaacagatccatgatggttagcaattgcttgtttcgtgttggtggggcagcgattttgctctccaacaaactgggagatcggagacggtccaagtacaagctatgtcatactgttcgaacgcataccggagctgatgacaagtcttttcgatgtgtgcaacaaggagacgatgagggcggtaaaatcggagtttgtctgtcaaaggacataaccgttgttgcggggacagcgcttaagaaaaacatagcaacgttgggtccgttgattcttcctttaagcgaaaagtttctgtttttagttaccttcatcgccaagaaacttttgaaggacaagatcaagcactgttacgtcccggatttcaagcttgctatcgaccatttctgtattcatgcgggaggcagagccgtgatcgatgtgcttgagaagagcttaggactatcgccaatcgatgtggaggcatctagatcaacgttacatagatttgggaatacttcgtctagctcaatttggtatgaattggcatacatagaagcaaaaggaaggatgaagaaagggaatagagcttggcagattgctttagggtcagggtttaagtgtaacagtgcggtttgggtggctctatgcaatgtcaaggcttcggcgaatagtccttgggaagattgcatcgatagatatccggttcaaattgattctgattcatcaaaatcagagactcatgtcaaaaacggtcggtcctaa

SEQ ID NO: 72 - DNA, 3583 bp, Camelina sativa
gaggcgtctagaatgactttgcatagatttggaaacacttcctcgagctcgatatggtatgaattggcttacacggaagctaaaggaagaatgaggaaagggaacagagtttggcagattgcttttggaagcgggtttaagtgtaacagcgcggtttggatggctctccgcgatgtcgagccctcgtttaaaatccttgggaacattgcatcgatagatatccggttaagatcgatctttgaactcgtaagaacggtggattggtccggaaacatggttagtcctccatgtaccaaaaaagaaagtgaacttatatctctagttctttactaaggggtcaagaaatcagttgaaggttaatgtatgtttatatatgttgtgtataaaggtaacccttaaatgaccaagactgttgacctattaaaatttaaaatttaagttctctcgaaaaattaaatcgtttgtaaaataaaaattgtgagttagaaaaatcaaaacaatttacttacctacaagaaccttaaaattaaacttaagtattaaattttgaatacatgaaaaaatgttaaacttatgacaccaaaatttacgaactctgaggacggaaatgcttaattcagtttgtttgtttttttgttttgtaaatcaatctggtttgttttcaatgtatctaaactaaagtcgggttatgacaccaacaccaagcttatggagacaggatgttaagtcgggttagtcatcaaggtattcaaataagtaataacttttaagttttagaacttttttgtcagtaaaatattttgtataataaaaaagtttagttaaaacattaaaacagtttttactgaaaatattatttgtgaaaaaacttcttttaagtgttaaatttagaacacgtaaaaacatgttagacttatgacaccaagcttatagacccgaaaaggaaactgtgattgtttccttttccccacgagttctaagttgtttcctttacttatttgggttgcaaccaatataaaaagaaagaaaaatggaaatttatcacaaaaaggagagtcgagatctctagagttacattcatatgtgctagtgtctgaccttttgccttaatttataggcactaataaaaaaactaccaaaaaaaaaattgaagaagaacaaaaggttaaacaatcactggatcagtgtgctcatggtgttccaggtccaaaacatgtgtcacgagattaaaaaagagagaaacacaactgaaactattcaccaagatcacaactatatatataaaggacttataaaaaatcaatttgaaagtggttaaactaaacgattataagtgtgattgcacttacccccttataggtttggtgaatcttattagagataacttatttttaagatagttgcaattaaaaaaaaaaaaaaattgtccggatagtttgatgcaattaatgagtgttctatatggtctgattcttaatatttatgcaaatattatagtatttcaaagtatttggccttaactgacagactttgcaaataaaactcattcccgagaaacattcatcccataattggtatttagtcagaggctaatcgactatggcctttcagccaatcaaagctacgaacacgaatccccctaaaacatcctcaagtatttatttaatacatcgtattgagcaccactcataaactaattccatacatttatcatactgtttatttgtaataataaaagcagcaacatattgtagtttgtaggcaataagaaacaaaacaaaacatttttttttctctccaaattttcaaaattggaaaacgaaacttggaccttcaatacttatatattatatttgcaatataaaattgcagagtggactatttcccttattttggcaactttcagtggactagtaatttatttcaatgtgtatgcttgcatgagtgtgaatatacacatgtctatatgcatgcctgcaaatcgtaacggaccacaaaaaaggatccatacaaatatacctctcaacggctcctctctattatgctctccgacacaaactgagaaatgacgtccgttaacgcaaagctcctttaccattacgtcctaaccaactttttcaacctttgcttgtttccgttaacggcgttacttgccggaaaagcctctacgcttaccacaaacgatctctaccacttctattcccatctccaacacaaccttgtaaccgtaattttactctttgctttctcctctttcggtttggttctctacgttgtaacccggcgcagaccggtttacctcgttgactactcgtgctaccttccaccaccgcatctcaaagttagtgtttctaaggtcatggatattttctaccaaataagaaaagctgatacctcacgaaacgtggcatgcgatgatccatcctcgcttgatttcctgaggaagattcaagaacgttcaggtctaggtgatgaaacctacagtcccccgggactcattcacgtgcccccacaaaagacttttgcagcttcacgtgaagagacagagcaggtaatcatcggtgcgttagaaaagttattcgagaacaccaaagttaaccctagagagattggtatacttgtggtcaactcaagcatgtttaatccaactccttcgctatctgcgatggtcgttaacactttcaagctccgaagcaacatcaaaagctttagtctcggaggaatgggttgtagtgctggtgtcatcgccattgatcttgcaaaggacttgttgcatgttcataaacacacttatgcacttgtggtgagcactgagaacatcactcaaggcatttatgctggcgaaaatagatccatgatggttagcaattgcttgtttcgtgttggtggggcagcgattttgctatccaacaagccgggagatcggagacggtccaagtacaagctatgtcacacggttcggactcataccggagctgatgacaagtcttttcgatgtgtgcaacaaggagacgatgagagcggtaaaatcggagtttgtctgtcaaaggacataacagttgttgcggggacagcgcttaagaaaaacatagcaacgttaggtccgttgattcttcctttaagcgaaaagtttctgttcttagttaccttcatcgccaagaaacttttgaaggacaagatcaagcactattacgtcccggatttcaagcttgctattgaccatttctgtattcatgccggaggcagagccgtaatcgatgtgcttgagaagagcttaggactatcgccaatcgatgtggaggcatctagatcaacgttacatagatttgggaatacttcgtctagctcaatttggtatgaattggcatacatagaagcaaaaggaaggatgaagaaagggaatagagcttggcagattgctttagggtcagggtttaagtgtaacagtgcggtttgggtggctctatgcaatgtcaaggcttccgcgaatagtccttgggaacattgcatcgatagatatccggttcaaattgattctgattcatcaaaatcagagactcatgtcaaaaacggtcggtcctaa

SEQ ID NO: 73 - DNA, 3447 bp, Camelina sativa
aaccccaaaaaattcatcaaaccctttcttcttctcaccacatttttcctctttcactgagcccattttgagatcctctctctttctctctctctctctctctctttctccggagatggttgttgctatggacaaacgtagcaatgtgaacggagatcccggcgccggagaccggaacaagaaaggagaagggtttgatccgagtgcacagcctccgttcaagatcggtgatataagggcggcaataccaaaacactgttgggttaagagtcctttgagatcaatgagttacgtcgtcagagacatttgcgccgtcgctgctcttgccgtcgccgccgtgtattttaacagctggttcctttggcctctttattgggccgctcaaggcaccctttactgggccatcttcgtcctcggccacgactggtatttttttttttttttttttttactaatgttacgttttcaaataatttaatttaattaattatttttcgatttagtttttgtgactttttttgcttttttaattgcatcgaattcttaaattatactttgaatttgcacgttcgatgaagtattgtcgctaacttcaatatttgttcttttgaggttactttaatttgttaaaaaactgaaaaagaggatccaagatttctgtttttagattttttataagttttcacttttcagtaacgttccactatgatgattagtcgtaagactcgtaacaaaatatctaaagacttttctattattattaataataccgcatttcgtggatctacgttccaacttcacaaattactattgtttcttttagatctacggtttttttttttttttaattattcatgccttcatggaattattgaattgttaatataacgtaccaatgtactttcgagatttatacacgtcatatatatatattttctaacatttcttgatgcgttttaaatatttttgccagtatattataagatttataggtatttaatgaataatgatgtgaaattgaatgtgtaaaaataatgacacagtggacatgggagtttctcagacattcctctgctgaatagtgtggttggtcacattcttcattccttcatcctcgttccttaccatggttggtaagttatttacttttattcttttctctctttttttttttttttttagtgttacgttgttatttttggtgtatttgatgcatgttaggaatttaatagataaccttcacttcaatcgtactttttttttttggggtgtattgcaaattttgtgattagaaaataatgcatgtgactattatgattatgaggtggcttttgatttttccactccaaaagcatcaaatcttttagtaaccaaccaagagggcatcaaaacttttattaaatgttgtgggccaatttatggcccattagccaatctgaacatgaagaaattgatgtacctttgtctaattaacattaattttgtgaaagccaaatgtgtgtttttttttttttttttttttgggtttgagattgagattgatgtcctaaatgtattcatcggctgatatcattaaaaagtagatttgtatgcatcattatagtactgtttgtttagaatctgccgcatagagttgtgtataacttgtatttatagaacaaaacaagaactaattttttgtgaatttatataattggggttgtaggagaataagccaccggacgcaccaccagaaccatggccatgttgaaaacgacgagtcatgggttccggtagtctttccttctctcatctgtgttctcttgtcgtttttttgttttttttttctttcctatattcgaatttttatatgttagcgtactcctagaatatgttttttttttccttcaatagtatgttttatttatagatttagtttaggcaagttgaaatcctaacaacaaccacttggtcaagtgtcgtttgttgaatcagtaattgatttttttttttttggtttatagaatcaactcttttcaaaatataatactactaatatataactttaatagtggaccacttatattcgggtccttcgcataggacgattctcctcgaagtgcgaatactttcttttcatttttttttttctcttcttggcgttttcgtgatattttgtctagatcttgaaaaaaatacaaaaaaaaaaattagaatgcacatgcaatgtataatgataacgattaaaaccatagtgtaaatgtatacgagtgtataaattaataacaaaagtcattttaatcaaaacatttcagttaccagaaagggtgtacaagaacttgccccacagtactcggatgctgagatacactgtccctctccccatgcttgcttatcctctctatctggtaaatgtttttaaaacctagtcttgcacaataattgatttttagtttttttaaagtaataaaagttgatattggattttttttttttgacagtgctacagaagtcctggaaaggaaggatcacattttaacccatacagtagtttatttgctccaagcgagagaaagcttattgcaacttcaactacttgttggtccataatgtttgtcactcttattgctctgtcattcatcttcggtccactttccgttcttaaagtctacggtgtgccgtacatcgtaagttatttcatatctttatactgtgtgagcaattttatgatatacatgtttttgacataaattttggatttatttgttttttcagatctttgtgatgtggttggacgctgtcacgtatttacatcaccatggtcacgatgagaagttgccttggtacagaggcaaggtaatttaaaagaaaaaaatcaactatatttatgtttataagaaaacaaaaatctctagaaatattagcaatgtgttgtataggaatggagtaattgatttgtttggaaaatataggaatggagttatctacgtggaggactaacaactattgatagagattacggaatcttcaacaacattcatcacgacattggaactcacgtgatccatcatctcttcccacagatccctcactatcacttggtcgatgccgtgagtggatcatttcattatacatatactgagattagctctaataaaagggggtgattaactaattagtgattgtttaatttgaatgattgacagacaaaatcagctaaacatgtgttgggaagatactacagagaaccacagacgtcaggagcaataccgatccacttggtggagagtttggtcgcaagtattaagaaagatcattacgtcagtgacactggtgatattgtcttctacgagacagatccagatctctacgtttacgcttctgacaagtctaaaatcaattaaatttcttcctttgtttagctctattaggaataaaccagcaagtccccccttttttttttaattt

SEQ ID NO: 74 - DNA, 3447 bp, Camelina sativa; identical to SEQ ID NO: 73

SEQ ID NO: 75 - DNA, 3441 bp, Camelina sativa
tcttcttctcaccacattttttctttctttcactgagagcccatttctctttctctctctttctctctttctccggagatggttgttgctatggacaaacgtagcaatgtgaacggagatcccggcgccggagaccagaagaagaaaggagaagggtttgatccgagtgcacagcctccgttcaagatcggtgatataagggcggcaataccaaaacactgttgggttaagagtcctttgagatcaatgagttacgtcgtcagagacatttgcgccgtcgctgctcttgccgtcgccgccgtgtattttaacagctggttcctttggcctctttattgggccgctcaaggcacccttttctgggccatcttcgtcctcggccacgactggtaatttttttttactaatcttacgtttttaaataatttaatttaattaatttaatttttcaatttagtttttgcttttttaattgcattgaattcttaaattatactttgaatttgcacgttcgatgaagtattgtcgctaacttcaatatttgttcttttgaggttactttgatttgttaaaaaacagaaaaagaggatccaagatttctgtttttagattttttttttatttttcaataacgttccactatgatggttagtcgtaagactcgtaacaaaatatctaaagacttttctattattattaataataccgcatttcatggatctacgttcgaactttcacaaattactattgtttcttttagatctacggggttttttttttaattgttcatggaactattgaattattaatataatgtaccaatgtactttcgagatttatacacgtcatatatagatatattttctaacctttcttgaggcgttttatattatgaaaacatggacaaatattaatatttttgcagtatatattataagatgtatgtgtgtttaatgaaatattgatgtgaaattgaatgtgaaaaaataatgacacagtggacatgggagtttctcagacattcctctgctgaatagtgtggttggtcacattcttcattccttcatcctcgttccttaccatggttggtaagttatttacttttattcttttctcttattactattattttttactgtttacgttgttatttttggtgtatttggtgcatgttaggaatttaatctgataaccttcatttcaatcggatatttttttttgggatgtattgcaaattttgtgattagaaaataaagcatgtgactatatgaggtggcttttgatttttccactccaaaagcatcaaatcttttagtaaccaaccaaaaaaagagggcatccaaacttttattaaatgttgtgggccaatttatggcccgttagccaacctgaacatgaagaaattgatgtacctttgtctaattaacattaattttgtgaaaatgtgtttttttttttagattgagattgatgtcctaaatgtatccatcggctggtatctttaaaaagtagatttgtatgcatcattataagaaacattatagtactgtttgtttgttacttaccccctcctagttataaattttgaagattgttagaatctgccacaaagacttgtgtatacttttgtttatagaacttaacaaaaactaattttttgtgaatttatacaattgggttgtaggagaataagccaccggacacaccaccagaaccatggccatgttgaaaacgacgagtcatgggttccggtaatctttccttctctcatctgtctactcttgtctttttttctttcctatattcgaatttttatatgctagctaactcctaggatatgttttaattttattttattatgttttagttaatagatttagtttaggcaagttgaatcctaacaacaaccacttggtcagttttattttattttcaagtgtcgtttgttgaatcagtaattgatttttttttttgtggtttatagaaggatcaactctgtcaacatataatactactaataatatataactttaatagtggaccacttatattcgggtccttcgcataggacgattctcctcgaagtgcgaatacttttctttttcatttttttctcttcttggcgttttcgtaatattttgtctagatcttgaaaaaaatactaatttttttttagaaaaattagaatgcacatgcgctgtataatgataacgattaaaaccataatgtaaatttatacgagtgtataaattaataacagtcattttaatcaaaacatttcagttaccagaaagggtgtacaagaacttgccccacagtactcggatgctgagatacactgtccctctccccatgcttgcttatcctctctatctggtaagtatttttaaatcctagtcttgcacaataattgattttttagattttttaaagtaataaaagttgatattggatttttattttgacagtgctacagaagtcctggaaaggaaggatcacattttaacccatacagtagtttatttgctccaagcgagagaaagcttattgcaacttcaactacttgttggtccataatgtttgtcactcttattgctctatcattcatcttcggtccactctccgttcttaaagtctatggtgtgccgtacatcgtaagttatttcataactttatactgtgtgagcaattttatgatatacatgtttttgacataaattttggatttatttgtttttttcagatctttgtgatgtggttggacgctgtcacgtatttacatcaccatggtcacgatgagaagttgccttggtacagaggcaaggtaatttttttaaaaaattcaactatatttatgtttgtaagaaaacaaaaatctctagaaatattagcaatgttatataggaatggagtaattgatttgtttgaaaaatataggaatggagttatctacgtggaggactaacaactattgatagagattacggaatcttcaacaacattcatcacgacattggaactcacgtgatccatcatctcttcccacagatccctcactatcacttggtcgatgccgtgagtggatcatttttttatttttactgagattagttctaatcctaataataaaagggaggggtgattaattaaattagtgatttgttttatttgaatgattgacagacaaaagcagctaaacatgtgttgggaagatactacagagaaccacaaacgtcaggagcaataccgatccacttggtggagagtttggtcgcaagtattaagaaagatcattacgtcagtgacactggtgatattgtcttctacgagacagatccagatctctacgtttacgcttctgacaagtctaaaatcaattaaatctcttcctttgtttagctctattaggaataaaccagcaagtccccctttttttaattt

SEQ ID NO: 76 - DNA, 5250 bp, Camelina sativa
  misc_feature (629)..(629): n is a, c, g, or t
  misc_feature (1701)..(1701): n is a, c, g, or t
gtgttttgttgtattctcatctctttctcttctctctctctctctctctctgtctctgtttgttttggcgtctccacgcacgattttacgcatcgaaggctctctgcacgcctcctgaaagagagagagagagagaaggagagagagatcatcacagatcgattctctcttaaatctctcgtgaatcccatttccccttccgctagattctctcttcactcatttctcgctttctctccttttgttctctcttatctctctctctcttcgcttcattcactttttttttccttgctatggtggtgggtgcgtcttcttgttacgcatctccgttatgcacttggttcgttgctgcttgcatgtccgtctctcacgacggcggcggagatagccgccaggctgctgttgctcttcaatctggtgggcggagtcggcgaaggaggcagcttaccaaatgctctgcctctgcctctcgatccccttcccctcctaccattcaagctcttgtctcttcttgtttggattttggtccctgtactcactactccaactccaacaacaatgccttgtcttctctctttggatccagtactctttctttgaatcgacaccagaggagattgaatcgnaaataaaacaaaataaaataatggtattcccagtgtcctccttccttcgattaaaccgaggtcgccaaatctctctgtatgcacttttgtgtaaataaaataaaataaattgtatcaacataaaataatgattttctctataatttatgattggatacaatacaaatatgtgtaaatgaatcactatttcatcattctaatgcactcttttcagtgtttcttttttttttttcctttttttttgataagatggcatcaattttaacaataaaaaaggttgcaaaagttcattataaataaaaataaataaaaaatttattctggtcatcgataatattgagatttgaagtgtcgattggtataattatttttgtgtatacctgtgtagagtgttttgttgtattctcatctctttctcttctctctctctctctctctctgtctctgtttgttttggcgtctccacgcacgattttacgcatcgaaggctctctgcacgcctcctgaaagagagagagagagagaaggagagagagatcatcacagatcgattctctcttaaatctctcgtgaatcccatttccccttccgctagattctctcttcactcatttctcgctttctctccttttgttctctcttatctctctctctcttcgcttcattcactttttttttccttgctatggtggtgggtgcgtcttcttgttacgcatctccgttatgcacttggttcgttgctgcttgcatgtccgtctctcacgacggcggcggagatagccgccaggctgctgttgctcttcaatctggtgggcggagtcggcgaaggaggcagcttaccaaatgctctgcctctgcctctcgatccccttcccctcctaccattcaagctcttgtctcttcttgtttggattttggtccctgtactcactactccaactccaacaacaatgccttgtcttctctctttggatccagtactctttctttgaatcgacaccagaggagattgaatcgagctacttcttcttcttcttccggtatttacatttctcttccaatctgggttttnttttttttttttttttttttttttttatgttccttagttcgatgttgctatggggtttaatggtattgggctaatgatggaagcagctcacatgtgaaaagtttggattttttatcttcattccatgtgttattatgtttcaaaagaaggcacttttttttgtgaggttaggggctctagatgaggaatgagggatacttaacaagtggcggtgaactatatataatataaattttgaagttgcctttagctattttgtttaccataccattgagatgtggtttctgtaatagctgatttaccatcaactgtaatgtctttgtggcttttaggtggagccatggcagtgatggagatggaaaaggaagccatggttaacaagaaaccacctacggagcagcgtcgggttgtagtgacaggcatgggagttgaaacaccgttaggtcatgacccacatactttttatgagaatttgctacaaggcaacagtggtattagccagattgagaattttgattgttctgaatttcctacggtaagcattattatatcaggatctctcttggaagctttatattgtgtgaagtttctccatttttgctaatgttttgtatttgaatatcttttccttgttttacctttgtcaagcgaattgcgggagagatcaaaagcttctcgactgaaggatgggttgctccaaaactttcgaaaaggatggacaagttcatgctctatcttctcacagctggcaagaaagctttggctgatggtggggtaactgaagaagtaatggcagagtttgacaaaaccaaatgtggagttttgattggctcagcaatgggagggatgaaggtactttcttgatcccgggcttgaatttaaggtttttgttctgtatctgactaacaaaagagtcacatgaatattgaatctataaattagggagtctgttgcttattgttgcattttctaataatctagtattagtacaggtcttttacgatgctattgaagctctgagaatctcatacaagaagatgaatcctttttgtgtaccttttgcgacaacaaacatgggttctgctatgcttgctatggatctggtctgttaaatttcaaatgttatataatttgatatggtcgaccaccagcaatttcatactgacttttctttgtggtgatacatcagggatggatggggccaaactattctatttcaactgcttgtgccacaagcaacttttgcattctgaattcagcaaaccacattattaaaggcgaagctgtaagtattctccatccacttggtcattcgacagttcggtagacttcttctggttctacttatagactgtctatctttccactaaacttttcaggatgtaatgctctgtggtggttcagatgcagttattattccaataggtgcttactcgttctcttatattacattatcccctctattctccatatttaagctacgtgaaatcttatcgtatgtgtaccaatgcagggttgggaggttttgttgcatgccgggctctttcacaaaggaataatgatcccacaaaagcttcacgtccttgggatagtgtaatttttttacaatttcttgtctcgtcttctttttctttctgtacatatgctgtagtatccttctgtggttttgttttaatcttgattgccaatggtggatgctagaaacactgtatcaagtttctaccaacaggaatttcccttacatgtctgtaatgttgtagtaggaaagataatgagaggaaatgtgtgtattatgtttggagattcttcttgttttgttaacctggtcttgtatgcttatcagaatcgagatggttttgtgatgggagagggagctggagttctacttttggaagaacttgagcatgctaaggtatatatccatttgttctttttgtcattccttgtttcctattattttatcacaaattaaacttatctccaaaccattttttaactcttgttatggaacgagttgtaagaacttaagtattttattctgcttttctttcagaaaagaggagcaactatctacgcagagttcctcggtgggagtttcacatgtgatgcgtatcacatgaccgagcctcaccctgatggtagtttcatttctccctcatattagtctgctagtcaactacaaattttaaatctcttttttttctattaattttgtcattatcccatttttcttatatcacaggtgctggtgtcatcctctgtatcgagagagcgttagcgagtgctggagtctcaagggaacagataaattacataaatgcacatgcaacctcaacacacgctggagaccttagggagtaccaagctcttgttaactgttttggccaaaatcctgaggtagcttcacttctctgttacaaaactccaaggtggctttttgtgtgcttttctttaacttgatctttcacgatgtggcagcttaaagtaaattccacaaaatctatgattggacacttgctgggagctgctggggccgtggaggctgttgcaaccattcaggtaacacatttgatatttgagacatcagtatatctacaaggaacgaaacgctgttgaaaccaagtttcataacagagcgaaattatgtgcaatcttcgcattttttatgttaatgttctgttgttacctacaggcaataaggaccggatgggttcatccgaatatcaacctcgagaatccagacagtggagtggtatgttagtaatattgttggcttctgttattccattctgatcgtatgagttcaatacctacacaaagactaccatttaatgttttgaaatgaaatggaacaggatacaaagctgatggtgggtcctaagaaggagagattaaacattaaagcagcattgtcaaattctttcgggtttggtggccataactcgagcattattttcgctccttacaagtgaagaacgaaggctgttgcttgtattccaaacctggttgtataacttgctgtaaggtacattacatacattgttcattgatgcatcatcaagttttgagaggatgcaaaaggaaaatttactcctttgaccttaacgtatttattactctgattgagatggatcattgacaagaatgaattgaagtttgtcggtccagaaattcatttaatgatttgggatgactaggattctttgtactgtgtctgttatgaatatgagtgattgttttgatatgtgtgcagtgtttacaagaactttcccatgttaagctagtgttacgttgcgggaatcaacagagtttgttcaactaccaagagctaaagctaagtttcttaggatcaagatctgatgagccaaagacttggataggagcaaaagttgccagagatagcagagtttggatttccattaaaaaaaaaaacaaatcttgttcatcgtgataccttcttattggggaaatctttgtagtccttcttttctattattttaacatacaatctcaaaaaatgccaaatcaattctcagttaaatttagtaa

SEQ ID NO: 77 - DNA, 5358 bp, Camelina sativa
  misc_feature (3381)..(3381): n is a, c, g, or t
ttgtattcatctcttctctctctctttctctctgtctctgtttgttgtttttttatcagaggaggatttttggcgtctccacgcacgattttacgcatcgaaggctctctgcacgcctcctgaaagagagagagagagagaagaagagagagagatcatcacagatcgattctctcttaaatctctcgtgaataccatttccccttccgctagattctctcttcactcatttctcgctttctctccttttgttctctctctctctctctcaccttctctcttcgcttcattcactttttttcttgccatggtggtgggtgcgtcttcttgttacgcatctccgttatgcacttggttcgttgctgcttgcatgtccgtctctcacgacggcggcggtggagatagccgtcaggctgctgttgcgcttcaatctggtgggcggagtcggcgaaggaggcagcttaccaaatgctcctctgcctcttctcctcctcgatccccttcccctcccaccattcaagctcttgtctcttcttgtttggattttggtccttgtactcactactccaacaccaacaacaatgccttgtcttctctctttggatccaatactctttctgtgaatcgaaaccagaggagattgaatcgagctactccttcttccggtatttacatttctcttccaatctgggttttttttttttctttaatgttccttagttcgatgttgctatgggtttatggtattgggctaatgatggaagcagctgacatgtgaaagtttggatttttatcttgatttcatgtattattaaagaaggtactttttcgtgtggttaggctctagatgaggaatcagggatactgaacaagtggcggtgaactatataatatgaattctgatgttgcctgtaactatcttgtttaccataccattgagatgttgtttcggagtaagctaatttaccatcaactgtaatgtctttgtggtctttaggtggagccatggcagtgatggagatggaaaaggaagccatggttaacaagaaaccgcctacggagcagcgtcgggttgtagtgacaggcatgggagttgaaacaccattaggtcatgacccacataccttttatgagaatttgctacaaggcaacagtggtattagccagattgagaattttgattgttctgaatttcctacggtaagcattattatatcaggatctcttgcaagctttatattgtgtgaaatttctccattttgctattgttttgtattttaatatctttccttggttttacctttgtcaagcgcattgcgggagagatcaaaagcttctcgactgaaggatgggttgctccaaaactttcgaaaaggatggacaagttcatgctctatcttctcacagctggcaagaaagctttggctgatggtggggtaactgaagaagtaatggcagagtttgacaaaaccaaatgtggagttttgattggctcagcaatgggaggaatgaaggtactttcttgatcccgggcttgaaacagtttcaatttaaggttttttgttcagtatctgactaacaaaaaagtcacatgaatattgaatctataaattagggagtctgttgcttattgttgcgtttctaataatctaatattagtacaggtcttttacgatgctattgaagctctgagaatctcatacaagaagatgaatcctttttgtgtgccttttgcaacaacaaacatgggttctgctatgctagctatggatctggtctgactagaatttcaaatgttatataatttaatatggacgaccacaagcaacttcagactgacgtttctttgtggtaataaatcagggatggatggggccaaactattctatttcaactgcttgtgccacaagcaacttttgcattctgaattcagcaaaccacattattaaaggcgaagctgtaagtaataagtattctccatccacttggtaattcgacagttcggtagacttcttctggttctacttatagactgtctgtctttccactaaaattttcaggatgtgatgctctgtggtggttcagatgcagttattattccaataggtgctaactcgttctcttatattacagtattccctctattcttgatatttaagctacgtgaaatcttatcgtatgtgtaccaatgcagggttgggaggttttgttgcatgccgggctctttcacaaaggaataatgatcccacaaaagcttcacgcccttgggatagtgtaaacttttttacaatttcttgtctccgtcttcttttccttttgtacttgcatgtgtagcgttactctatggttttgttttaatcttgattatcgagtttccgccaacaggaatttcccttgtatgtgtgtaatgttgtagggaagataatgggaggaaatgtgtgtaatatgtatggagaatcttcttattttgttaacttggtcttgatttcatatcagaatcgagatggtttcgtgatgggagagggagctggagttctacttttggaagaacttgaacatgctaaggtatataattcatttgttctttttgtctttccttgtttcccattgtttaatcacaaatgaaacttgtctgcaaaccttttttttattattcttgctatggaacgagctgtaagagcttaagtattctgcttttctttcagaaaagaggagcaactatctacgcagagttcctcggtgggagtttcacatgtgatgcgtatcacatgaccgagcctcaccctgatggtagtttcatttttccctacttcatattagtctgctcgtcaacttcaaattctaaatctcttttttactattaattttttcattatcccatttttcttatatcacaggtgctggtgtcatcctctgtatcgagagagcgttagctagtgctggagtctcaagggaacagataaattacataaatgcacatgcaacctcaacacacgctggagaccttagggagtaccaagcccttgttaattgttttggccaaaaccctgaggtagcttcacttctctgttacaaaactccaaggtggctttttgtatgattttctttaacttgatctttcacgatgtggcagcttaaagtaaattccacaaaatctatgattggacacttgctgggagctgctggggccgtggaggctgttgcaactattcaggtaacagattttatctttgagatatcagtatatctacaaggaacgaaacgctgttgaaaccaagtttcataacagagtgaaactatgtgcaatattcgcattttaaatgttagttttctgttgttacctacaggcaataaggaccggatgggttcatccaaatatcncccttgggatagtgtaaacttttttacaatttcttgtctccgtcttcttttccttttgtacttgcatgtgtagcgttactctatggttttgttttaatcttgattatcgagtttccgccaacaggaatttcccttgtatgtgtgtaatgttgtagggaagataatgggaggaaatgtgtgtaatatgtatggagaatcttcttattttgttaacttggtcttgatttcatatcagaatcgagatggtttcgtgatgggagagggagctggagttctacttttggaagaacttgaacatgctaaggtatataattcatttgttctttttgtctttccttgtttcccattgtttaatcacaaatgaaacttgtctgcaaaccttttttttattattcttgctatggaacgagctgtaagagcttaagtattctgcttttctttcagaaaagaggagcaactatctacgcagagttcctcggtgggagtttcacatgtgatgcgtatcacatgaccgagcctcaccctgatggtagtttcatttttccctacttcatattagtctgctcgtcaacttcaaattctaaatctcttttttactattaattttttcattatcccatttttcttatatcacaggtgctggtgtcatcctctgtatcgagagagcgttagcgagtgctggagtctcaagggaacagataaattacataaatgcacatgcaacctcaacacacgctggagaccttagggagtaccaagcccttgttaattgttttggccaaaaccctgaggtagcttcacttctctgttacaaaactccaaggtggctttttgtatgattttctttaacttgatctttcacgatgtggcagcttaaagtaaattccacaaaatctatgattggacacttgctgggagctgctggggccgtggaggctgttgcaactattcaggtaacagattttatctttgagatatcagtatatctacaaggaacgaaacgctgttgaaaccaagtttcataacagagtgaaactatgtgcaatattcgcattttaaatgttagttttctgttgttacctacaggcaataaggaccggatgggttcatccaaatatcaacctcgagaatccagacagtggagtggtatgttagtaatttttttgttggcttctgttattccattctgatcgtatgagttcaatacctacacaaagagcaccatttaatgttttgaaatgaaactggaacaggatacaaagctgatggtgggtccgaagaaggagagattaaacattaaagcagccttgtcaaattctttcgggtttggtggccataactcgagcattattttcgctccttacaaatgaaaaacgaaggctgttgcttgtattccaaacctggttgtataacttgctgtaaggtacattacatacattgttcattgatgcatcatcaagttttgagaggatgcaaaggacaatttagtcttttgacctttaacatattcattactctggttgagatggatcattgacaagaatgaattgaagtttgtctgcccagaaatgcatttaatgatttgggttgtacttatgagttatgatttctttgtactgtgtatctgttatgaagatgagtgcatgttttgacatgtgtgcagtgtttacaagaactttcccatgttaagctagtgttacgttgcgggaatcaacagagtttgttcaactaccaagagctaaagctaagtttcttttaggatcaagatctgatgagccaaagacttggataggagcaaaagttgccagagatagcggagtttggatttgccattaaaaaaaaaacaaatcttgttcatcgtgataccttcttcttattgggaaatctttgtagtccttcttttctattattttaacatacaatctgaaaaaaaaatgccaaatcaattctcagttaaatttagtaactcttgacagt

SEQ ID NO: 78 - DNA, 4232 bp, Camelina sativa
ttctctctctctccgtctctgtttgttttgttgtttttttttatcagaggagggacgatatttggcgtctccacgcacgattttacgcatcgaaggctctctgcacgcctcctgaaagagagagagagaaggagagagagatcatcacagatcgattctctcttaaatctctcgtgaatcccatttccccttccgctagattctctcttcactcacttctcgctttctctccttttgctctctctctctctctccttctctcttcgcttcattcactttttttttcttttgccatggtggtgggtgcgtcttcttgttacgcatctccgttatgcacctggttcgttgcagcttgcatgtccgtctctcacgaaggcggagatagccgccaggctgctgttgcgcttcaatctggtgggcggagtcggcgaaggaggcagcttactaaatgctcctctgcctcttctcctcctcgatccccttcccctcctcctaccattcaagctcttgtctcttcttgtttggattttggtccttgtactcactactccaactccaactccaacaacaatgccttgtcttctctctttggatccaatcctctttctttgaatcgacaccagaggagattgaatcgagctgcttctccttcttccggtatttacatttctcttccaatctgggttgttgtttttgtctgccaagttttagtctttcttcgatttttctcgatgttgctatgggtttatggtattgggctaatgatggaagcagctcacatgtgaaaaagtttggatttttatcgtgattcccatgtgttattattatgtttcaaaagaagactctttttgtgtgtggttaggctctagatgaggaatcagggatacatacagagcgtcgatatctgaacaagtggcggtgaactatataatataaaattttgaagttgcctgtaactattttgtttaccataccattcagatgttgtttctgagtaagctaatttaccatcaactgtaatgtctttgtggcttttaggtggagccatggcagtgatggagatggaaaaggaagccatggttaacaagaaaccacctacggagcagcgtcgggttgtagtgacaggcatgggagttgaaacaccattaggtcatgacccacataccttttatgagaatttgctacaaggcaacagtggtattagccagattgagaattttgattgttctgaatttcctacggtaagcattattattatattagcatctcttggaagctttatattgtgtgaaatctccccgtttgctattgttttgtatttgaatatctttccttggtttacctttgtcaagcgaattgcgggagagatcaaaagcttctcgactgaaggatgggttgctcctaaactttctaaaaggatggacaagttcatgctctatcttctcacagctggcaagaaagctttggctgatggtggggtaactgaagaagtaatggcagagtttgacaaaaccaaatgtggagttttgattggctcagcaatgggagggatgaaggtactttcttgatccgggcttgaacgtatagaaagtatgaaaacagtttcaatttaaggtttttgctcagtatctgactatcaaaagagtgacatgaatattaaatctaaatattagggagtctgttgcttattgttgcatttctaataatcttaatatattagtacaggtcttttacgatgctattgaagctctgagaatctcatacaagaagatgaatcctttttgtgtaccttttgcgacaacgaacatgggttctgctatgcttgctatggatctggtctgattgaatttcaaatgttttataatttaatatggacgaccacctgcaatctcagactgacttttctttgtggtgataaatcagggatggatggggccaaactattctatttcaactgcttgtgccacaagcaacttttgcattctgaattcagctaaccacattattaaaggcgaagctgtaagtattctccatccacttgggtaattcgacagtttggtagacttcttctggttctacttatagactgtctgtctttcctcgaaacttttcaggatgtaatgctctgtggtggttcagatgcagttattattccaataggtgcttggtcgttctcgtatattacattattccctctattcttgatatttaagccagcgaaatctaatcgtatgtgttccaatgcaggtttgggaggttttgttgcatgccgggctctttcacaaaggaataatgatcccacaaaagcttcacgtccttgggatagtgtaatttttttacaatttcttgtctcgtcttcttttccttttgtacttgcatgttgtagcatccttctgtggttttgttttaatcttgattgccaacggtggatgctagaaacactgtatcaagtttctgccaacaggaaatttcccttatatgtctgtaatgttgtagggaagattatgggaggaaatgtgtgtattatgtttggagattcttctcgttttgttaacctggtcttgtttgcttatcagaatcgagatggttttgtgatgggagagggagctggagttctacttttggaagaacttgagcatgctaaggtatataatttatttgctctttttttcattccttgtttctctaaacattttgtttattcttgctatggaacgagctgtaagaacttaagtattctgtttttctttcagaaaagaggagcaactatctacgcagagttcctcggtgggagtttcacatgtgatgcgtatcacatgaccgagcctcaccctgatggtagttacattttctcgtaattcatattagtctgctatttttctacaaattttaaatcccatttttcttatatcacaggtgctggtgtcatcctctgtatcgagagagcgttagcgagtgctggagtctcaagggaacagataaattacataaatgcacatgcaacctcaacacacgctggagaccttagggagtaccaagcccttgttaattgttttggccaaaatcctgaggtagcttcacttctctgtgacaaaactccaaggtggctttttgtgtgcttttctttaacttgatctttcacgatgtggcagcttaaagtaaattccacaaaatctatgattggacacttgctgggagctgctggggctgtggaggctgttgcaaccattcaggtaacacatttgatatttgagacatcagtatataaacaaggaacgaaacgctgttgaaaccaagtttcataacggagcgaaattatgtgcaatattcgcatttttaatgttagttttctgttgttacctacaggcaataaggaccggatgggttcatccaaatatcaacctcgagaatccagacagtggagtggtatgttagtaaattttgttgatttctgttattccattctgatcgtatgagttcaatacctacacaaagactaccatttaatgttttgaaatgaaatggaacaggatacaaagctgatggtgggtcctaagaaggagagattaaacattaaagcagccttgtcaaattctttcgggtttggtggccataactcgagcattattttcgctccttacaaatgaaaaacgaaggctgttgcttgtattccaaacctggttgtataacttgctgtaaggtacattacatacattgttcattgatgcatcatcaagttttgagaggatgcaaaggacaatttagtcttttgaccttaacacattcattactctggttaagatggatcattgacaagaatgaattgaagtttgtctgtccagaaatgcatttaatgatttggggtgtacttaagagttatgatttctttgtactgtatctgttatgaagatgagtgcatgttttgttatgtgtgcagtgtttacaagaactttcccatgttaagctagtgttacgttgcgggaatcaacagagtttgttcaactaccaagagctaaagctaagtttcttaggatcaagatctgatggccaaagacttggataggagcaaaagttgccagagatagcagagtttggatttgccataaaaaaaaaacatatcttgttcatcgtgataccttctttttattgggaaatctttgtagtccttcttttctattattttaacatacaatctgaacaaaaaatgccaaatcaattctcagttaaattta

SEQ ID NO: 79 - DNA, 1458 bp, Camelina sativa
atgacgtccgttaacgcaaagctcctttaccattacgtcctaaccaactttttcaacctttgcttgtttccgttaacggcgttacttgccggaaaagcctctaggcttacctcaaacgatctctaccacttctattcccatctccaacacaaccttataaccgtaattttactctttgctttcaccgctttcggtttggttctctacattgtaacccggcccaaaccggtttacctcgttgactactcgtgctaccttccaccaccgcatctcaaagttagtgtttccaaggcgatggatattttctaccaaataagaaaagctgatacctcacggaacgtggcatgcgatgatccatcctcgcttgatttcctgaggaagattcaagaacgttcaggtctaggtgatgaaacctacagtccccagggactcattaacgtgcccccacgaaagacctttgcagcttcacgtgaagagacagagcaggtaatcatcggtgcgctagataagctattcgagaataccaaagttaaccctagagaaattggtaatactttcaagcttcgaagcaacatcaaaagctttagtctcggaggaatgggttgtagtgctggtgtcatcgccattgatcttgcaaaggacttgttgcatgttcataaaaacacttatgcacttgtggtgagcactgagaacatcactcaaggcatttatgctggcgaaaatagatccatgatggttagcaattgcttgttccgtgttggtggcgcagcgattttgctctccaacaagccaggagatcggagacggtccaagtacaagttatgtcatactgttcgaacgcataccggagctgatgacatgtcttttcgatgtgtgcaacaaggagacgatgagagcggtaaaatcggagtttgtctgtcaaaggacataaccgttgttgcggggatagcgcttaagaaaaacatagcaacgttgggtccgttgattcttcctttaagcgaaaaatttctgtttttagtaaccttcatcgccaagaaacttttgaaggacaagatcaagcactattacgtcccggatttcaagcttgctattgaccatttctgtattcatgcgggaggcagagccgtgatcgatgtgcttgagaagagcttaggactatctccaatcgatgtggaggcatctagatcaacgttacacagatttgggaatacttcgtctagctcaatttggtatgaattggcatacatagaagcaaaaggaaggatgaagaaagggaatagagcttggcagattgctttagggtcaggatttaagtgtaacagtgcggtttgggtggctctatgcaatgtcaaggcttcggcgaatagtccttgggaacattgcatcgatagatatccggttcaaattgattctggttcatcaaaatcagatactcatgtcaaaaacggtcggtcctaa

SEQ ID NO: 80 - DNA, 1458 bp, Artificial sequence
  Camelina sativa mutation
atgacgtccgttaacgcaaagctcctttaccattacgtcctaaccaactttttcaacctttgcttgtttccgttaacggcgttacttgccggaaaagcctctaggcttacctcaaacgatctctaccacttctattcccatctccaacacaaccttataaccgtaattttactctttgctttcaccgctttcggtttggttctctacattgtaacccggcccaaaccggtttacctcgttgactactcgtgctaccttccaccaccgcatctcaaagttagtgtttccaaggcgatggatattttctaccaaataagaaaagctgatacctcacggaacgtggcatgcgatgatccatcctcgcttgatttcctgaggaagattcaagaacgttcaggtctaggtgatgaaacctacagtctccagggactcattaacgtgcccccacgaaagacctttgcagcttcacgtgaagagacagagcaggtaatcatcggtgcgctagataagctattcgagaataccaaagttaaccctagagaaattggtaatactttcaagcttcgaagcaacatcaaaagctttagtctcggaggaatgggttgtagtgctggtgtcatcgccattgatcttgcaaaggacttgttgcatgttcataaaaacacttatgcacttgtggtgagcactgagaacatcactcaaggcatttatgctggcgaaaatagatccatgatggttagcaattgcttgttccgtgttggtggcgcagcgattttgctctccaacaagccaggagatcggagacggtccaagtacaagttatgtcatactgttcgaacgcataccggagctgatgacatgtcttttcgatgtgtgcaacaaggagacgatgagagcggtaaaatcggagtttgtctgtcaaaggacataaccgttgttgcggggatagcgcttaagaaaaacatagcaacgttgggtccgttgattcttcctttaagcgaaaaatttctgtttttagtaaccttcatcgccaagaaacttttgaaggacaagatcaagcactattacgtcccggatttcaagcttgctattgaccatttctgtattcatgcgggaggcagagccgtgatcgatgtgcttgagaagagcttaggactatctccaatcgatgtggaggcatctagatcaacgttacacagatttgggaatacttcgtctagctcaatttggtatgaattggcatacatagaagcaaaaggaaggatgaagaaagggaatagagcttggcagattgctttagggtcaggatttaagtgtaacagtgcggtttgggtggctctatgcaatgtcaaggcttcggcgaatagtccttgggaacattgcatcgatagatatccggttcaaattgattctggttcatcaaaatcagatactcatgtcaaaaacggtcggtcctaa

SEQ ID NO: 81 - DNA, 1458 bp, Camelina sativa
atgacgtccgttaacgcaaagctcctttaccattacgtcctaaccaactttttcaacctttgcttgtttccgttaacggcgttacttgccggaaaagcctctaggcttacctcaaacgatctctaccacttctattcccatctccaacacaaccttataaccgtaattttactctttgctttcaccgctttcggtttggttctctacattgtaacccggcccaaaccggtttacctcgttgactactcgtgctaccttccaccaccgcatctcaaagttagtgtttccaaggcgatggatattttctaccaaataagaaaagctgatacctcacggaacgtggcatgcgatgatccatcctcgcttgatttcctgaggaagattcaagaacgttcaggtctaggtgatgaaacctacagtccccagggactcattaacgtgcccccacgaaagacctttgcagcttcacgtgaagagacagagcaggtaatcatcggtgcgctagataagctattcgagaataccaaagttaaccctagagaaattggtatacttgtggtcaactcaagcatgtttaatccaactccttcgctatctgcgatggtcgttaatactttcaagcttcgaagcaacatcaaaagctttagtctcggaggaatgggttgtagtgctggtgtcatcgccattgatcttgcaaaggacttgttgcatgttcataaaaacacttatgcacttgtggtgagcactgagaacatcactcaaggcatttatgctggcgaaaatagatccatgatggttagcaattgcttgttccgtgttggtggcgcagcgattttgctctccaacaagccaggagatcggagacggtccaagtacaagttatgtcatactgttcgaacgcataccggagctgatgacatgtcttttcgatgtgtgcaacaaggagacgatgagagcggtaaaatcggagcgttgggtccgttgattcttcctttaagcgaaaaatttctgtttttagtaaccttcatcgccaagaaacttttgaaggacaagatcaagcactattacgtcccggatttcaagcttgctattgaccatttctgtattcatgcgggaggcagagccgtgatcgatgtgcttgagaagagcttaggactatctccaatcgatgtggaggcatctagatcaacgttacacagatttgggaatacttcgtctagctcaatttggtatgaattggcatacatagaagcaaaaggaaggatgaagaaagggaatagagcttggcagattgctttagggtcaggatttaagtgtaacagtgcggtttgggtggctctatgcaatgtcaaggcttcggcgaatagtccttgggaacattgcatcgatagatatccggttcaaattgattctggttcatcaaaatcagatactcatgtcaaaaacggtcggtcctaa

SEQ ID NO: 82 - DNA, 1458 bp, Artificial sequence
  Camelina sativa mutation
atgacgtccgttaacgcaaagctcctttaccattacgtcctaaccaactttttcaacctttgcttgtttccgttaacggcgttacttgccggaaaagcctctaggcttacctcaaacgatctctaccacttctattcccatctccaacacaaccttataaccgtaattttactctttgctttcaccgctttcggtttggttctctacattgtaacccggcccaaaccggtttacctcgttgactactcgtgctaccttccaccaccgcatctcaaagttagtgtttccaaggcgatggatattttctaccaaataagaaaagctgatacctcacggaacgtggcatgcgatgatccatcctcgcttgatttcctgaggaagattcaagaacgttcaggtctaggtgatgaaacctacagtccccagggactcattaacgtgcccccacgaaagacctttgcagcttcacgtgaagagacagagcaggtaatcatcggtgcgctagataagctattcgagaataccaaagttaaccctagagaaattggtatacttgtggtcaactcaagcatgtttaatccaactccttcgctatctgcgatggtcgttaatactttcaagctttgaagcaacatcaaaagctttagtctcggaggaatgggttgtagtgctggtgtcatcgccattgatcttgcaaaggacttgttgcatgttcataaaaacacttatgcacttgtggtgagcactgagaacatcactcaaggcatttatgctggcgaaaatagatccatgatggttagcaattgcttgttccgtgttggtggcgcagcgattttgctctccaacaagccaggagatcggagacggtccaagtacaagttatgtcatactgttcgaacgcataccggagctgatgacatgtcttttcgatgtgtgcaacaaggagacgatgagagcggtaaaatcggagcgttgggtccgttgattcttcctttaagcgaaaaatttctgtttttagtaaccttcatcgccaagaaacttttgaaggacaagatcaagcactattacgtcccggatttcaagcttgctattgaccatttctgtattcatgcgggaggcagagccgtgatcgatgtgcttgagaagagcttaggactatctccaatcgatgtggaggcatctagatcaacgttacacagatttgggaatacttcgtctagctcaatttggtatgaattggcatacatagaagcaaaaggaaggatgaagaaagggaatagagcttggcagattgctttagggtcaggatttaagtgtaacagtgcggtttgggtggctctatgcaatgtcaaggcttcggcgaatagtccttgggaacattgcatcgatagatatccggttcaaattgattctggttcatcaaaatcagatactcatgtcaaaaacggtcggtcctaa

SEQ ID NO: 83 - DNA, 1155 bp, Camelina sativa
atgggtgcaggtggaagaatgccagttccttcttcttcttccaagaaatctgaaaccgatgccataaagcgtgtgccctgcgagaaaccaccgttcacgctgggagatctgaagaaagcaatcccaccgcagtgtttcaaacgctctatccctcgctctttctcctaccttatcactgacatcattattgcctcctgcttctactacgtcgccaccaattacttttctctcctccctcagcctctctcttacttggcttggcccctctattgggcttgtcaaggctgtgtcctaaccggtgtctgggtcatagcccacgaatgcggtcaccacgcattcagcgactaccagtggctcgatgacacagtcggtcttatcttccattccttccttctcgtcccttacttctcctggaagtacagtcatcgccgtcaccattccaacacaggatccctcgaaagagatgaagtatttgtcccaaagcagaagtccgctatcaagtggtatggcaaatacctcaacaaccctgctggacgcatcatgatgttgaccgtccagtttgtcctcgggtggcccttgtacttggcctttaacgtctccggcagaccatacgacgggttcgcttgccatttcttccccaacgctcccatctacaacgaccgtgaacgcctccagatatatctctctgatgccggtattctagcagtctgttttgggctttaccgttacgccgctgcacaaggattggcctcgatgatctgcctctacggagtaccacttctgatagtgaacgcgttcctcgtcttgatcacttacttgcagcacactcatcctgcgttgcctcactacgattcatccgagtgggattggcttaggggagctttggctaccgtagacagagactatggaatcttgaataaggtgttccacaacatcacggacacacatgtggctcatcatctgttctcgacaatgccgcattataatgcgatggaagctacaaaggcgataaagccaatactcggtgactattaccagttcgacggaacaccatggtatgtggccatgtatagggaggcaaaggagtgtatctatgtagaaccggacagggaaggtgacaagaaaggtgtgtactggtacaacaataagttatga

SEQ ID NO: 84 - DNA, 1155 bp, Artificial sequence
  Camelina sativa mutation
atgggtgcaggtggaagaatgccagttccttcttcttcttccaagaaatctgaaaccgatgccataaagcgtgtgccctgcgagaaaccaccgttcacgctgggagatctgaagaaagcaatcccaccgcagtgtttcaaacgctctatccctcgctctttctcctaccttatcactgacatcattattgcctcctgcttctactacgtcgccaccaattacttttctctcctccctcagcctctctcttacttggcttggcccctctattgggcttgtcaaggctgtgtcctaaccggtgtctgggtcatagcccacgaatgcggtcaccacgcattcagcgactaccagtggctcgatgacacagtcggtcttatcttccattccttccttctcgtcccttacttctcctggaagtacagtcatcgccgtcaccattccaacacagaatccctcgaaagagatgaagtatttgtcccaaagcagaagtccgctatcaagtggtatggcaaatacctcaacaaccctgctggacgcatcatgatgttgaccgtccagtttgtcctcgggtggcccttgtacttggcctttaacgtctccggcagaccatacgacgggttcgcttgccatttcttccccaacgctcccatctacaacgaccgtgaacgcctccagatatatctctctgatgccggtattctagcagtctgttttgggctttaccgttacgccgctgcacaaggattggcctcgatgatctgcctctacggagtaccacttctgatagtgaacgcgttcctcgtcttgatcacttacttgcagcacactcatcctgcgttgcctcactacgattcatccgagtgggattggcttaggggagctttggctaccgtagacagagactatggaatcttgaataaggtgttccacaacatcacggacacacatgtggctcatcatctgttctcgacaatgccgcattataatgcgatggaagctacaaaggcgataaagccaatactcggtgactattaccagttcgacggaacaccatggtatgtggccatgtatagggaggcaaaggagtgtatctatgtagaaccggacagggaaggtgacaagaaaggtgtgtactggtacaacaataagttatga

SEQ ID NO: 85 - DNA, 1164 bp, Camelina sativa
atggttgttgctatggacaaacgtagcaatgtgaacggagatcccggcgccggagaccggaacaagaaaggagaagggtttgatccgagtgcacagcctccgttcaagatcggtgatataagggcggcaataccaaaacactgttgggttaagagtcctttgagatcaatgagttacgtcgtcagagacatttgcgccgtcgctgctcttgccgtcgccgccgtgtattttaacagctggttcctttggcctctttattgggccgctcaaggcaccctttactgggccatcttcgtcctcggccacgactgtggacatgggagtttctcagacattcctctgctgaatagtgtggttggtcacattcttcattccttcatcctcgttccttaccatggttggagaataagccaccggacgcaccaccagaaccatggccatgttgaaaacgacgagtcatgggttccgttaccagaaagggtgtacaagaacttgccccacagtactcggatgctgagatacactgtccctctccccatgcttgcttatcctctctatctgtgctacagaagtcctggaaaggaaggatcacattttaacccatacagtagtttatttgctccaagcgagagaaagcttattgcaacttcaactacttgttggtccataatgtttgtcactcttattgctctgtcattcatcttcggtccactttccgttcttaaagtctacggtgtgccgtacatcatctttgtgatgtggttggacgctgtcacgtatttacatcaccatggtcacgatgagaagttgccttggtacagaggcaaggaatggagttatctacgtggaggactaacaactattgatagagattacggaatcttcaacaacattcatcacgacattggaactcacgtgatccatcatctcttcccacagatccctcactatcacttggtcgatgccacaaaatcagctaaacatgtgttgggaagatactacagagaaccacagacgtcaggagcaataccgatccacttggtggagagtttggtcgcaagtattaagaaagatcattacgtcagtgacactggtgatattgtcttctacgagacagatccagatctctacgtttacgcttctgacaagtctaaaatcaattaa

SEQ ID NO: 86 - DNA, 1164 bp, Artificial sequence
  Camelina sativa mutation
atggttgttgctatggacaaacgtagcaatgtgaacggagatcccggcgccggagaccggaacaagaaaggagaagggtttgatccgagtgcacagcctccgttcaagatcggtgatataagggcggcaataccaaaacactgttgggttaagagtcctttgagatcaatgagttacgtcgtcagagacatttgcgccgtcgctgctcttgccgtcgccgccgtgtattttaacagctggttcctttggcctctttattgggccgctcaaggcaccctttactgggccatcttcgtcctcagccacgactgtggacatgggagtttctcagacattcctctgctgaatagtgtggttggtcacattcttcattccttcatcctcgttccttaccatggttggagaataagccaccggacgcaccaccagaaccatggccatgttgaaaacgacgagtcatgggttccgttaccagaaagggtgtacaagaacttgccccacagtactcggatgctgagatacactgtccctctccccatgcttgcttatcctctctatctgtgctacagaagtcctggaaaggaaggatcacattttaacccatacagtagtttatttgctccaagcgagagaaagcttattgcaacttcaactacttgttggtccataatgtttgtcactcttattgctctgtcattcatcttcggtccactttccgttcttaaagtctacggtgtgccgtacatcatctttgtgatgtggttggacgctgtcacgtatttacatcaccatggtcacgatgagaagttgccttggtacagaggcaaggaatggagttatctacgtggaggactaacaactattgatagagattacggaatcttcaacaacattcatcacgacattggaactcacgtgatccatcatctcttcccacagatccctcactatcacttggtcgatgccacaaaatcagctaaacatgtgttgggaagatactacagagaaccacagacgtcaggagcaataccgatccacttggtggagagtttggtcgcaagtattaagaaagatcattacgtcagtgacactggtgatattgtcttctacgagacagatccagatctctacgtttacgcttctgacaagtctaaaatcaattaa

SEQ ID NO: 87 - DNA, 21 bp, Camelina sativa
tacagtccccagggactcatt

SEQ ID NO: 88 - DNA, 21 bp, Artificial sequence
  Camelina sativa mutation
tacagtctccagggactcatt

SEQ ID NO: 89 - DNA, 21 bp, Camelina sativa
tacagtcccccgggactcatt

SEQ ID NO: 90 - DNA, 21 bp, Arabidopsis thaliana
tacagtcctgagggactcatt

SEQ ID NO: 91 - DNA, 21 bp, Camelina sativa
actttcaagcttcgaagcaac

SEQ ID NO: 92 - DNA, 21 bp, Camelina sativa
actttcaagctccgaagcaac

SEQ ID NO: 93 - DNA, 21 bp, Artificial sequence
  Camelina sativa mutant
actttcaagctctgaagcaac

SEQ ID NO: 94 - DNA, 21 bp, Camelina sativa
ccaacacaggatccctcgaaa

SEQ ID NO: 95 - DNA, 21 bp, Artificial sequence
  Camelina sativa mutant
ccaacacagaatccctcgaaa

SEQ ID NO: 96 - DNA, 21 bp, Camelina sativa
cttcgtcctcggccacgactg

SEQ ID NO: 97 - DNA, 21 bp, Artificial sequence
  Camelina sativa  mutant
cttcgtcctcagccacgactg

SEQ ID NO: 98 - DNA, 21 bp, Arabidopsis thaliana
ctttgttctcggccacgactg